(19) United States
(12) Patent Application Publication
(10) Pub. No.: US 2004/0171823 A1
(43) Pub. Date: Sep. 2, 2004

(54) Polynucleotides and polypeptides associated with the NF-kappaB pathway

(76) Inventors: Steven G. Nadler, Princeton, NJ (US); Michael G. Neubauer, Skillman, NJ (US); John N. Feder, Belle Mead, NJ (US); Julie Carman, Lawrenceville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/755,889

(22) Filed: Jan. 13, 2004

Related U.S. Application Data
(60) Provisional application No. 60/440,068, filed on Jan. 14, 2003.
(60) Provisional application No. 60/469,757, filed on May 12, 2003.

(51) Int. Cl. C12Q 1/68; C07H 21/04

(52) U.S. Cl. 536/23.2; 435/6

(57) ABSTRACT

The present invention relates to polynucleotide and polypeptide sequences newly identified as associated with the NF-κB pathway. The identification of such polynucleotides and polypeptides is an important advancement toward discovering and identifying new drug targets for the treatment of NF-κB pathway-related diseases, disorders, and conditions. The invention further relates to compositions and methods for the treatment of diseases or disorders associated with the NF-κB signaling pathway using the sequences of the invention.

This application claims benefit to provisional application U.S. Ser. No. 60/440,068 filed Jan. 14, 2003; and to provisional application U.S. Serial No. 60/469,757, filed May 12, 2003; under 35 U.S.C. 119(e). The entire teachings of the referenced applications are incorporated herein by reference.

### FIELD OF THE INVENTION

The present invention relates to polynucleotide and polypeptide sequences newly identified as associated with the NF-κB pathway. In particular, this invention relates to the discovery of polynucleotides and polypeptides that are associated with regulating, i.e. decreasing or increasing, NF-κB pathway activity, either directly or indirectly. The polynucleotides and polypeptides of the invention serve as new targets for discovering and identifying protein modulators, e.g. drugs, compounds or biological agents for the treatment of NF-κB pathway-related diseases, disorders, and/or conditions. The invention further relates to compositions and methods for the treatment and prevention of diseases or disorders associated with the NF-κB pathway.

### BACKGROUND OF THE INVENTION

The Nuclear Factor-κB signaling pathway (NF-κB pathway) is a critical mediator of intracellular signaling and gene expression in virtually all cell types. NF-κB is composed of dimeric complexes of p50 (NF-κB1) or p52 (NF-κB2) that are usually associated with members of the Rel family (p65, c-Rel, Rel B) which have potent transactivation domains. Different combinations of NF-κB/Rel proteins bind to distinct κB sites to regulate the transcription of different genes. Early work involving NF-κB suggested that its expression was limited to specific cell types, particularly in stimulating the transcription of genes encoding kappa immunoglobulins in B lymphocytes. However, it has been discovered that NF-κB is, in fact, present and inducible in many, if not all, cell types and that it acts as an intracellular messenger capable of playing a broad role in gene regulation as a mediator of inducible signal transduction. Specifically, it has been demonstrated that NF-κB plays a central role in the regulation of intercellular signals in many cell types. For example, NF-κB has been shown to positively regulate the human beta-interferon (beta-IFN) gene in many, if not all, cell types. Moreover, NF-κB has also been shown to serve the important function of acting as an intracellular transducer of external influences.

As a transcriptional activator, NF-κB plays a central role in regulating the transcription of a number of genes, including those which encode proteins involved in inflammatory and immune responses. Representative examples of genes controlled by NF-κB include the cytokines tumor necrosis factor (TNF-α), IL-1β, IL-6, and IL-8; the adhesion molecules E-selecting and vascular cell adhesion molecule (VCAM)-1; and the enzyme nitric oxide (NO)-synthase (for reviews, see Siebenlist et al. Annu. Rev. Cell Biol. 10: 405-455, 1994; Bauerle and Baltimore, Cell, 87:13-20, 1997). Also, NF-κB has been shown to be induced by several stimuli, in addition to mediators of immune function, such as UV irradiation, growth factors, and viral infection.

The NF-κB transcription factor normally resides in the cytoplasm in unstimulated cells as an inactive complex with a member of the inhibitor κB (IκB) inhibitory protein family. The IκB class of proteins includes IκB-α, IκB-β, and IκB-ε—all of which contain ankyrin repeats for complexing with NF-κB (for review, see Whiteside et al., EMBO J. 16:1413-1426,1997). In the case of IκB-α, the most carefully studied member of this class, stimulation of cells with agents which activate NF-κB-dependent gene transcription results in the phosphorylation of IκB-α at serine-32 and serine-36 (Brown et al. Science, 267:1485-1488, 1995).

IκB is a cytoplasmic protein that controls NF-κB activity by retaining NF-κB in the cytoplasm. IκB is phosphorylated by the IκB kinase (IKK), which has two isoforms, IKK-1 (or IκB kinase α, IKKα) and IKK-2 (or IκB kinase β, IKKβ.). Upon phosphorylation of IκB by IKK, NF-κB is rapidly released into the cell and translocates to the nucleus where it binds to the promoters of many genes and up-regulates the transcription of pro-inflammatory genes. Inhibitors of IKK can block the phosphorylation of IκB and further downstream effects, specifically those associated with NF-κB transcription factors.

Aberrant NF-κB activity is associated with a number of human diseases. Mutations or truncations of IκB have been observed in some Hodgkins lymphomas (Cabannes et al. (1999) Oncogene 18:3063-3070). Genes encoding p65, p105, and p100 have been reported to be overexpressed or rearranged in some solid and hematopoietic tumors (Rayet et al. (1999) Oncogene 18:6938-6947). Missense mutations in IKKγ have been seen in some hyper-IgM syndromes characterized by hypohydrotic ectodermal dysplasia (Jain et al. (2001) Nature Immunol. 2:223-228), and in cases of X-linked anhidrotic ectodermal dysplasia with immunodeficiency (Doffinger et al. (2001) Nature Genet. 27:277-285). Genome rearrangements in IKKγ have also been observed in cases of familial incontinentia pigmenti (The International Incontinentia Pigmenti Consortium (2000) Nature 405:466-472).

In addition to the above genetic diseases, NF-κB is involved in many viral infections (Hiscott et al. (2001) J. Clin. Invest. 107:143-151). Several families of viruses including HIV-1, HTLV-1, hepatitis B, hepatitis C, EBV, and influenza activate NF-κB. The mechanisms of activation are distinct, and in some cases have not been well characterized. Some viral proteins have been identified that activate NF-κB including influenza virus hemagglutinin, matrix protein, and nucleoprotein; hepatitis B nucleoprotein and HBx protein; hepatitis C core protein; HTLV-1 Tax protein; HIV-1 Tat protein; and EBV LMP1 protein. The activation of NF-κB in target cells facilitates viral replication, host cell survival, and evasion of immune responses.

Many inflammatory diseases are associated with constitutive nuclear NF-κB localization and transcriptional activity. NF-κB is activated in the inflamed synovium of rheumatoid arthritis patients (Marok et al. (1996) Arthritis Rheum. 39:583-591) and in animal models of arthritis (Miagkov et al. (1998) Proc. Natl. Acad. Sci. USA 95:13859-13864). Gene transfer of a dominant negative IkBα significantly inhibited TNFα secretion by human synoviocytes (Bondeson et al. (1999) Proc. Natl. Acad. Sci. USA 96:5668-5673). In animal models of inflammatory bowel disease, treatment with antisense p65 oligonucleotides significantly inhibited clinical and histological signs of colitis (Neurath et al. Nature Med. 2:998-1004). NF-κB has also been associated with other inflammatory diseases including asthma, atherosclerosis, cachexia, euthyroid sick syndrome, and stroke (Yamamoto et al. (2001) J. Clin. Invest. 107:135-142). Therefore, regulation of NF-κB and/or its activation pathway provides a means for treating a wide range of diseases. See also, e.g., Baldwin, 1996, “The NF-κB and IκB Proteins: New Discoveries and Insights,” Annual Rev. Immunol., Vol. 14:649-81; and Christman et al., 2000, “Impact of Basic Research on Tomorrow's Medicine, The Role of Nuclear Factor-κB in Pulmonary Diseases,” Chest, Vol. 117:1482-87.

### SUMMARY OF THE INVENTION

This invention relates to polynucleotide and polypeptide sequences that are newly identified as associated with the NF-κB pathway. In particular, this invention relates to the discovery of polynucleotides and polypeptides that are associated with, regulated in, or regulate, i.e. decrease or increase, NF-κB pathway activity. According to the present invention, the identification of such polynucleotides and polypeptides, signaling pathways and pathway components is an important step toward discovering and identifying new drug targets for the treatment and prevention of NF-κB pathway-related diseases, disorders, and conditions, as described herein.

In accordance with this invention, subtraction library and microarray methods were utilized to isolate and identify new proteins associated with the NF-κB pathway. According to this invention and the findings related thereto, the NF-κB pathway-associated polypeptides can serve as drug targets for NF-κB pathway-related diseases and conditions. In addition, the proteins can be utilized as described herein to identify and/or screen for modulators, e.g., agonists or antagonists, for use in methods and compositions for the prevention and treatment of NF-κB-related disorders. It is to be understood that throughout this disclosure, the present invention relates to methods and compositions suitable for the prevention, treatment and therapeutic intervention of the NF-κB pathway and related diseases, disorders and conditions.

In specific embodiments, the invention relates to the polynucleotide sequences set forth in Tables 1-6, as well as complementary sequences, sequence variants, mutants, fragments or portions thereof, as described herein. The present invention also encompasses nucleic acid probes and primers that are useful for assaying biological samples for the presence or expression of the proteins of the invention. In particular, this invention relates to methods of regulating activation of the NF-κB pathway in order to control expression of genes whose expression is regulated by NF-κB.

The invention further encompasses novel nucleic acid variants or mutations of the polynucleotides and polypeptides associated with the NF-κB pathway.

The present invention further relates to isolated proteins, polypeptides, peptides and antigenic epitopes thereof unique to, associated with, regulated in and/or which regulate the NF-κB pathway. In specific embodiments, the polypeptides or peptides comprise the amino acid sequences encoded by the polynucleotide sequences set forth in Tables 1-6, or portions thereof, as described herein. In addition, this invention encompasses isolated fusion proteins comprising the polypeptides or peptides encoded by the sequences set forth in Tables 1-6.

In another aspect, the present invention encompasses vectors or vector constructs, including expression vectors and cloning vectors, which contain the NF-κB pathway-associated nucleic acid sequences, or peptides encoding portions of the NF-κB pathway-associated nucleic acid sequences, or variants thereof, for the expression of the NF-κB pathway-associated nucleic acid molecule(s) in host organisms. The present invention also relates to host cells molecularly/genetically engineered to contain and/or express NF-κB pathway-associated nucleic acid molecules. Such host cells which express NF-κB pathway-associated polypeptides or peptides can be employed in screening assays as described herein, for example, to identify NF-κB pathway-associated polypeptide modulating compounds, and/or to assess the effect(s) of a variety of cell treatments and compounds on NF-κB pathway-associated polypeptide function or biological activity, which can include structural, biochemical, physiological, or biochemical functions in a cell. Further, host organisms that have been transformed with these nucleic acid molecules are also encompassed in the present invention, e.g., transgenic animals, particularly transgenic non-human animals, and particularly transgenic non-human mammals.

The present invention also relates isolated antibodies, including monoclonal and polyclonal antibodies, and antibody fragments, that are specifically reactive with the NF-κB-associated polypeptides, fusion proteins, variants, or portions thereof, as disclosed herein. In specific embodiments, monoclonal antibodies are prepared to be specifically reactive with the NF-κB-associated polypeptides, fusion proteins, variants, or portions thereof.

It is another aspect of the present invention to provide modulators of the NF-κKB-associated polypeptides and peptide targets that can affect the function or activity of the NF-κB associated polypeptides in a cell and modulate or affect NF-κB-mediated transcription and signal transduction. In addition, modulators of the NF-κB-associated polypeptides can affect downstream systems and molecules that are regulated by, or that interact with, the NF-κB-associated polypeptides in the cell. Such modulators can be used as therapeutics for the treatment of NF-κB pathway-related disorders. Modulators of the NF-κB-associated polypeptides include antagonists, agonists, inhibitors, ligands, and binding factors. Antagonists include compounds, materials, agents, drugs, and the like, that antagonize, inhibit, reduce, block, suppress, diminish, decrease, or eliminate NF-κB pathway-associated protein function and/or activity. Alternatively, agonist modulators of NF-κB pathway-associated polypeptides include compounds, materials, agents, drugs, and the like, that agonize, enhance, increase, augment, or amplify NF-κB pathway-associated protein function in a cell.

Antagonists and agonists of the present invention also include, for example, small molecules, large molecules and antibodies directed against the NF-κB pathway-associated polypeptides or peptides thereof. Antagonists and agonists of the invention also include nucleotide sequences, such as antisense, small interfering RNAs, and ribozyme molecules, and gene or regulatory sequence replacement constructs, that can be used to inhibit or enhance expression of the NF-κB pathway-associated polypeptide encoding nucleic acid molecules, or oligomeric portions thereof, such as peptide encoding nucleic acid fragments.

Yet another aspect of this invention provides methods and compositions, including pharmaceutical compositions, for the treatment and/or prevention of NF-κB pathway-related disorders and conditions. The compositions can comprise the modulators of the NF-κB pathway-associated polypeptides, or peptides thereof. Pharmaceutical compositions preferably comprising a pharmaceutically and/or physiologically acceptable diluent, excipient, or carrier (vehicle) are provided. The modulators can be employed alone, or in combination with other standard treatment regimens for NF-κB pathway-related diseases and/or conditions. Such methods and compositions are capable of modulating the level of NF-κB pathway-associated polypeptide gene expression and/or the level of activity of the NF-κB-associated polypeptide. The methods include, for example, modulating the expression of the NF-κB pathway-associated polypeptide, or modulating the expression of a gene or gene product that is regulated or controlled by the NF-κB-associated polypeptide, including NF-κB, effective for the treatment of NF-κB-related disorders.

It is another aspect of the invention to provide screening methods for the identification of compounds, materials, substances, drugs, and agents that modulate the expression of the NF-κB pathway-associated polypeptides and/or the activity of the NF-κB pathway-associated polypeptides. Such methods include, without limitation, assays that measure the effects of a test compound or agent on NF-κB pathway-associated polypeptide mRNA and/or gene product levels; assays that measure levels of NF-κB pathway-associated polypeptide activity or function; and assays that measure the levels or activities of molecules and/or systems that are regulated or mediated by NF-κB pathway-associated polypeptides, or modulators of such polypeptides, including, but not limited to, NF-κB.

It is another aspect of the present invention to provide the NF-κB pathway-associated polypeptides as components of the NF-κB signaling pathway and as affecting downstream cellular events, including NF-κB-mediated transcription and gene expression. Accordingly, downstream cellular events can be regulated via the activity of the NF-κB pathway-associated polypeptides using NF-κB pathway-associated polypeptide modulators, e.g., antagonists or agonists, such as antisense polynucleotides, polypeptides or low molecular weight chemicals to achieve a therapeutic effect in a broad variety of NF-κB pathway-related diseases including, but not limited to, proliferative disorders, cancers, ischemia-reperfusion injury, heart failure, immunocompromised conditions, HIV infection, hyper-IgM syndromes characterized by hypohydrotic ectodermal dysplasia, incontinentia pigmenti, inflammatory diseases including rheumatoid arthritis, osteoarthritis, inflammatory bowel disease, asthma, and chronic obstructive pulmonary disease, viral infections including HIV, HTLV-1, hepatitis B, hepatitis C, influenza, and EBV, atherosclerosis, cachexia, euthyroid sick syndrome, stroke, and renal diseases.

It is another aspect of this invention to provide NF-κB pathway-associated polynucleotides and polypeptides, and portions thereof, for treating, diagnosing, and/or ameliorating a broad variety of NF-κB pathway-related diseases including, but not limited to, proliferative disorders, cancers, ischemia-reperfusion injury, heart failure, immunocompromised conditions, HIV infection, and renal diseases. According to the invention, the NF-κB pathway-associated polynucleotides and polypeptides, and portions thereof, are useful for regulating NF-κB pathway activity.

It is another aspect of the present invention to provide antagonists or agonists directed against NF-κB pathway-associated polypeptides for treating, diagnosing, and/or ameliorating NF-κB pathway-related disorders including, but not limited to, proliferative disorders, cancers, ischemia-reperfusion injury, heart failure, immunocompromised conditions, HIV infection, and renal diseases. According to the present invention, antagonists or agonists directed against NF-κB-associated polypeptides are useful for regulating NF-κB pathway activity for diagnostic and therapeutic purposes.

In another aspect of the present invention, methods are provided for regulating second messenger pathways and molecules therein by modulating NF-κB pathway-associated polypeptide function and/or activity. More particularly, the present invention affords the ability to regulate, modulate, or affect the activity of the NF-κB pathway and components thereof by modulating, i.e. antagonizing or agonizing, the function and/or activity of the NF-κB pathway-associated polypeptides of the invention. NF-κB-associated polypeptide modulation can result in treatments for diseases and disorders that are mediated by NF-κB and/or other molecules related thereto. Accordingly, the present invention further provides methods of treating diseases that are caused by, or are associated with, the NF-κB pathway and/or its components, preferably in which antagonist or agonist modulators of NF-κB pathway-associated polypeptides are employed to decrease or increase the activity of the NF-κB pathway and/or its component molecules.

It is yet another aspect of the present invention to provide antisense nucleic acid molecules that specifically antagonize NF-κB pathway-associated nucleic acids, e.g., by binding to mRNA of NF-κB pathway-associated polypeptides or peptides. Antisense molecules refer to nucleotide sequences, e.g., oligomers, and compositions containing nucleic acid sequences that are complementary to a specific DNA or RNA sequence, such as NF-κB-associated polypeptide DNA or RNA sequences. Antisense moelcules may be single or double stranded.

An additional aspect of this invention pertains to the use of NF-κB pathway-associated polynucleotide sequences and antibodies including, monoclonal, polyclonal, and antibody fragments, directed against the produced polypeptides and peptides for diagnostic assessment of NF-κB pathway-related diseases or disorders.

Another aspect of the present invention relates to a method of diagnosing, ameliorating, treating, reducing, eliminating, or preventing a disease, disorder, and/or condition affected by modulation of the NF-κB pathway-associated-polypeptides in cells that express them, which involves providing a modulator, e.g., an agonist or antagonist, of the NF-κB-associated polypeptide in an amount effective to affect the function or activity of the polypeptide, and/or to effect the function or activity of cellular molecules that are associated or correlated with modulated polypeptide activity or function. Examples of diseases, disorders, and/or conditions that can be diagnosed, ameliorated, treated, reduced, eliminated, or prevented by the methods of this invention, in which NF-κB-associated polypeptides are modulated, include without limitation, proliferative disorders, cancers, ischemia-reperfusion injury, heart failure, immunocompromised conditions, HIV infection, hyper-IgM syndromes characterized by hypohydrotic ectodermal dysplasia, incontinentia pigmenti, inflammatory diseases including rheumatoid arthritis, osteoarthritis, inflammatory bowel disease, asthma, and chronic obstructive pulmonary disease, viral infections including HIV, HTLV-1, hepatitis B, hepatitis C, influenza, and EBV, atherosclerosis, cachexia, euthyroid sick syndrome, stroke, and renal diseases.

Further aspects, features, and advantages of the present invention will be better appreciated upon a reading of the detailed description of the invention when considered in connection with the accompanying figures or drawings.

### DESCRIPTION OF THE FIGURES

FIGS. 1A through 1DD show the real-time PCR results of newly identified sequences of the present invention that are inhibited by either NF-κB or the NF-κB pathway. RNA quantification was performed using the Taqman® real-time-PCR fluorogenic assay. FIG. 1A shows the results of CLK1 (Accession #L29219) (SEQ ID NOS: 1 & 2). FIG. 1B shows the results of Cytokine-Inducible Kinase (Accession #BC013899) (SEQ ID NOS: 3 & 4). FIG. 1C shows the results of GPR85 (Accession #AF250237) (SEQ ID NOS: 5 & 6). FIG. 1D shows the results of RGS16 (Accession #BC006243) (SEQ ID NOS: 7 & 8). FIG. 1E shows the results of SDCBP (Accession #BC013254) (SEQ ID NOS: 9 & 10). FIG. 1F shows the results of BTG1 (Accession #NM_{—}001731) (SEQ ID NOS: 11 & 12). FIG. 1G shows the results of JTB (Accession #NM_{—}006694) (SEQ ID NOS: 13 & 14). FIG. 1H shows the results of BCL2L11 (Accession #NM_{—}006538) (SEQ ID NOS: 15 & 16). FIG. 1I shows the results of BCL-6 (Accession #NM_{—}001706) (SEQ ID NOS: 17 & 18). FIG. 1J shows the results of EED (Accession #U90651) (SEQ ID NOS: 19 & 20). FIG. 1K shows the results of Similar to lysosomal amino acid transporter 1 (Accession #XM_{—}058449) (SEQ ID NOS: 21 & 22). FIG. 1L shows the results of Truncated Calcium Binding Protein (Accession #NM_{—}016175) (SEQ ID NOS: 23 & 24). FIG. 1M shows the results of WDR4 (Accession #AJ243913) (SEQ ID NOS: 25 & 26). FIG. 1N shows the results of FLJ22649 (Accession #NM_{—}021928) (SEQ ID NOS: 27 & 28). FIG. 10 shows the results of FLJ21313 (Accession #NM_{—}023927) (SEQ ID NOS: 29 & 30). FIG. 1P shows the results of MGC20791 (Accession #XM_{—}046111) (SEQ ID NOS: 31 & 32). FIG. 1Q shows the results of LOC113402 (Accession NM_{—}145169) (SEQ ID NOS: 33 & 34). FIG. 1R shows the results of DKFZp761I241 (Accession AL136565) (SEQ ID NOS: 35 & 36). FIG. 1S shows the results of DGCRK6 (Accession #AB050770) (SEQ ID NOS: 37 & 38). FIG. 1T shows the results of TNF-Induced Protein (Accession #BC007014) (SEQ ID NOS: 39 & 40). FIG. 1U shows the results of FLJ12120 (Accession #AK022182) (SEQ ID NOS: 747). FIG. 1V shows the results of GSA7 (Accession #NM_{—}006395) (SEQ ID NOS: 749 & 750). FIG. 1W shows the results of HSPC128 (Accession #NM_{—}014167) (SEQ ID NOS: 751 & 752). FIG. 1X shows the results of C2GNT3 (Accession #NM_{—}016591) (SEQ ID NOS: 753 & 754). FIG. 1Y shows the results of FLJ20512 (Accession #NM_{—}017854) (SEQ ID NOS: 755 & 756). FIG. 1Z shows the results of FLJ11715 (Accession #NM_{—}024564) (SEQ ID NOS: 757 & 758). FIG. 1AA shows the results of LNX (Accession #NM_{—}032622) (SEQ ID NOS: 759 & 760). FIG. 1BB shows the results of FLJ14547 (Accession #NM_{—}032804) (SEQ ID NOS: 761 & 762). FIG. 1CC shows the results of XBP1 (Accession #NM_{—}005080) (SEQ ID NOS: 763 & 764). FIG. 1DD shows the results of IL-23 alpha (IL23A)(Accession #NM_{—}016584) (SEQ ID NOS: 765 & 766).

FIGS. 2A through 2P show the real-time PCR results of newly identified sequences of the present invention that are induced by either NF-κB or the NF-κB pathway. RNA quantification was performed using the Taqmano real-time-PCR fluorogenic assay. FIG. 2A shows the results of SGKL (Accession #AF085233) (SEQ ID NOS: 41 & 42). FIG. 2B shows the results of KIAA0794 (Accession#AB018337) (SEQ ID NOS: 43 & 44). FIG. 2C shows the results of KIAA0456 (Accession #AB007925) (SEQ ID NOS: 45 & 46). FIG. 2D shows the results of ORPHAN NUCLEAR RECEPTOR TR4 (Accession #U10990) (SEQ ID NOS: 47 & 48). FIG. 2E shows the results of SUMO-1-specific protease (SUSP1, Accession #NM_{—}015571) (SEQ ID NOS: 49 & 50). FIG. 2F shows the results of SUMO-1 activating enzyme subunit 1 (Accession # NM_{—}005500) (SEQ ID NOS: 51 & 52). FIG. 2G shows the results of BRCA1-associated RING domain protein (BARD1, Accession #U76638) (SEQ. ID. NOS.: 53 & 54). FIG. 2H shows the results of MGC:4079 (Accession #BC005868) (SEQ ID NOS: 55 & 56). FIG. 2I shows the results FLJ23390 (Accession #AK027043) (SEQ ID NOS: 57 & 58). FIG. 2J shows the results of MGC19595 (Accession #NM_{—}033415) (SEQ ID NOS: 767 & 768). FIG. 2K shows the results of Gle1 (Accession #NM_{—}001499) (SEQ ID NOS: 769 & 770). FIG. 2L shows the results of BLVRA (Accession #NM_{—}000712) (SEQ ID NOS: 771 & 772). FIG. 2M shows the results of PPP1R7 (Accession #NM_{—}002712) (SEQ ID NOS: 773 & 774). FIG. 2N shows the results of MADH5 (Accession #NM_{—}005903) (SEQ ID NOS: 775 & 776). FIG. 2O shows the results of CHS1 (Accession #NM_{—}000081) (SEQ ID NOS: 777 & 778). FIG. 2P shows the results of ZNF304 (Accession #NM_{—}020657) (SEQ ID NOS: 779 & 780).

FIG. 3 shows the relationship of the Drosophila melanogaster Darkener of Apricot (DOA) gene to Human CDC-Like Kinase (CLK) genes.

FIG. 4 shows the effects of RNAi on NF-κB-dependent transcription.

FIGS. 5A through 5E show the effects of inhibitors of the NF-κB activation pathway on selected target genes. FIG. 5A shows inhibition or induction of Cytokine-Inducible Kinase (CNK) (Accession #BC013899) (SEQ ID NOS: 3 & 4) expression in the presence of the IKK-2 inhibitor, BMS-345541, or dexamethasone. FIG. 5B shows inhibition or induction of BCL-2 Like 11 (Accession #NM_{—}006538) (SEQ ID NOS: 15 & 16) expression in the presence of BMS-345541 or dexamethasone. FIG. 5C shows inhibition or induction of BCL-6 (Accession #NM_{—}001706) (SEQ ID NOS: 17 & 18) expression in the presence of BMS-345541 or dexamethasone. FIG. 5D shows inhibition or induction of MGC20791 (Accession #XM_{—}046111) (SEQ ID NOS: 31 & 32) expression in the presence of BMS-345541 or dexamethasone. FIG. 5E shows inhibition of Stat1 (Accession #NM_{—}007315) (SEQ ID NOS: 823 & 748) in the presence of BMS-345541 or dexamethasone.

FIGS. 6A through 6C show the NF-κB-dependent expression of selected target genes in mouse embryonic fibroblasts derived from germline knockouts of different NF-κB family members. FIG. 6A shows results of knockout experiments for Stat1 (Accession #NM_{—}007315) (SEQ ID NOS: 823 & 748). FIG. 6B shows results of knockout experiments for MGC20791 (Accession #XM_{—}046111) (SEQ ID NOS: 31 & 32). FIG. 6C shows results of knockout experiments for BCL-6 (Accession #NM_{—}001706) (SEQ ID NOS: 17 & 18).

FIG. 7A shows the level of transcriptional activity of NF-κB, TNFα, and IL-1β in an A549 cell line overexpressing the MGC20791 transcript, in addition to containing a stably integrated NF-κB reporter construct. As shown, overexpression of MGC20791 resulted in an increase in NF-κB-dependent transcriptional activity, but did not result in transcriptional increased activity in either TNFα or IL-1β. Experiments were performed as described in Example 12 herein.

FIG. 7B shows the protein level of MGC20791 expressed in A549 cells either in the presence (“MGC20791”) or absence (“CT”) of siRNA directed against MGC20791. Actin protein levels were used as a control. Protein levels were quantitated using anti-FLAG antibody to detect MGC20791 expression levels, and anti-actin antibody to normalize MGC20791 expression levels. As shown, MGC20791 expression levels were decreased in the presence of the MGC20791-directed siRNA reagents. Experiments were performed as described in Example 12 herein.

FIG. 8A shows the results of siRNA directed against MGC20791 on the levels in TNFα-induced and PMA/Ionomycin-induced NF-κB activation in A549 stable reporter cell lines. As shown, partial knockdown of the MGC20791 protein by siRNA resulted in decreased TNFα-induced and PMA/Ionomycin-induced NF-κB activation. Experiments were performed as described in Example 12 herein.

FIG. 8B shows the effect of siRNA directed against MGC20791 on TNFα-induced MCP-1 production by human umbilical vein endothelial cells (HUVEC). As shown, transfection of HUVECs with siRNA specific for MGC20791 significantly inhibited TNFα-dependent MCP-1 secretion at levels similar to the p65 subunit of NF-κB and the transcription factor Stat1. Experiments were performed as described in Example 12 herein.

### DETAILED DESCRIPTION OF THE INVENTION

The present invention identifies polynucleotide and polypeptide sequences that are associated with, regulated in, and/or regulate the NF-κB pathway. In particular, the present invention identifies new sequences that are regulated in or regulate, i.e. increase or decrease, NF-κB-dependent signal transduction and transcriptional activity. As stated above, the NF-κB pathway is now known to be a critical mediator of gene expression in a variety of cell types. For this reason, regulating or influencing transduction by NF-κB of extracellular signals, will enable one to selectively regulate the expression of proteins whose expression is mediated by NF-κB for the treatment of a broad variety of NF-κB-related diseases.

In accordance with the present invention, the proteins have been newly identified to be associated with the NF-κB pathway. Because of their first identification as proteins that regulate or influence NF-κB-dependent signaling and gene expression as described herein, the proteins emerge by virtue of the present invention as new targets for use in identifying protein modulators, e.g., drugs, compounds, or biological agents, and the like, of NF-κB-related cellular responses and for the treatment and prevention, of NF-κB-related diseases, disorders and conditions.

Briefly, to achieve the identification of polynucleotide and polypeptides associated with the NF-κB pathway, subtraction library and microarray assays were developed (Examples 1 and 4). Although the methods described above and in the Examples are the preferred methods for identifying protein targets that interact with NF-κB, such proteins may also be identified using alternative techniques well known in the art.

### Nucleic Acids and Variants

The present invention relates to the polynucleotide sequences shown in Tables 1-6 that are newly described by this invention as being involved in the NF-κB pathway, and in NF-κB-related diseases, disorders and conditions. Although the sequences are known in the art, they have not been previously shown to be associated with, or linked to, cellular responses associated with the NF-κB pathway, or NF-κB-related diseases, disorders and conditions.

As used herein, a polynucleotide or nucleic acid molecule or a nucleic acid can also refer to portions, fragments and/or degenerate variants of nucleic acid sequences, including naturally ocurring variants or mutant alleles thereof. Such portions or fragments include, for example, nucleic acid sequences that encode portions of the proteins identified in Tables 1-6 that correspond to functional domains of the proteins. In particular, a protein fragment, or peptide, as determined by the methods of the present invention is further embraced by the present invention.

The nucleic acid molecules as described herein can comprise the following sequences: (a) the DNA sequences shown in Tables 1-6 (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); (b) any nucleic acid sequences that encode the amino acid sequences shown in Tables 1-6 (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780); (c) any nucleic acid sequences that hybridizes to the complement of nucleic acid sequences that encode the amino acid sequences shown in Tables 1-6 under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (see, e.g., F. M. Ausubel et al., eds., 1989, Current Protocols in Molecular Biology, Vol. I, Green Publishing Associates, Inc., and John Wiley & sons, Inc., New York, at p. 2.10.3); or (d) any nucleic acid sequences that hybridizes to the complement of the nucleic acid sequences that encode the amino acid sequences shown in Tables 1-6 under less stringent conditions, such as moderately stringent conditions, e.g., washing in 0.2×SSC/0.1% SDS at 42° C. (F. M. Ausubel et al., 1989, supra), and which encodes a gene product functionally equivalent to a gene product encoded by the nucleic acid sequences depicted in Tables 1-6. Although the sequences identified in the Sequence Listing are preferred, the invention encompasses the sequences available through the corresponding accession numbers listed in Tables 1-6.

“Functionally equivalent” as used herein refers to any protein capable of exhibiting a substantially similar in vivo or in vitro activity as the gene products encoded by the nucleic acid molecules described herein, e.g., modulation of the NF-κB pathway or NF-κB-related diseases and conditions, or direct causative effects associated with NF-κB and related diseases and conditions.

As used herein, the term “nucleic acid molecule” or “nucleic acid” can also refer to portions, fragments and/or degenerate variants of the nucleic acid sequences of (a) through (d) above, including naturally occurring variants or mutant alleles thereof. Such fragments include, for example, nucleic acid sequences that encode portions of the proteins shown in Tables 1-6 that correspond to functional domains of the proteins. In addition, the nucleic acid molecules can include isolated nucleic acids, preferably DNA molecules, that hybridize under highly stringent or moderately stringent hybridization conditions to at least about 6, preferably at least about 12, more preferably at least about 18, and most preferably about 42 consecutive nucleotides of the nucleic acid sequences of (a) through (d), as described above.

In specific embodiments, the polynucleotides of the invention are at least 15, at least 30, at least 50, at least 100, at least 125, at least 500, at least 1000, at least 1500, at least 2000, at least 2500, at least 3000, at least 3500, or at least 4000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

The terms “stringent conditions” or “stringency” refer to the conditions for hybridization as defined by nucleic acid composition, salt, and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (for example, formamide, dextran sulfate and/or polyethylene glycol) and reaction temperature (within a range of from about 5° C. below the melting temperature of the probe to about 20° C.-25° C. below the melting temperature). One or more factors may be varied to generate conditions, either low or high stringency that are different from, but equivalent to, the aforementioned conditions.

As will be understood by those of skill in the art, the stringency of hybridization can be altered in order to identify or detect identical or related polynucleotide sequences. As will be further appreciated by the skilled practitioner, the melting temperature, Tₘ, can be approximated by the formulas that are well known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982; J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger,1987, Methods Enzymol. 152:399-407; and A. R. Kimmel, 1987; Methods of Enzymol. 152:507-511).

As a general guide, Tₘ decreases approximately 1° C.-1.5° C. with every 1% decrease in sequence homology in an aqueous solution containing 100 mM NaCl. Also, in general, the stability of a hybrid is a function of ionic strength and temperature. Typically, the hybridization reaction is initially performed under conditions of low stringency, followed by washes of varying, but higher stringency. Reference to hybridization stringency, for example, high, moderate, or low stringency, typically relates to such washing conditions. It is to be understood that the low, moderate and high stringency hybridization or washing conditions can be varied using a variety of ingredients, buffers and temperatures well known to and practiced by the skilled artisan.

The nucleic acid molecules of the invention can also include nucleic acids, preferably DNA molecules, that hybridize to, and are therefore complements of, the nucleic acid sequences of (a) through (d), as set forth above. Such hybridization conditions may be highly stringent or moderately stringent, as described above. In those instances in which the nucleic acid molecules are deoxyoligonucleotides (“oligos”), highly stringent conditions may include, e.g., washing in 6×SSC/0.05% sodium pyrophosphate at 37° C. (for 14-base oligos), 48° C. (for 17-base oligos), 55° C. (for 20-base oligos), and 60° C. (for 23-base oligos).

As will be discussed further below, the nucleic acid molecules of the invention can encode or act as antisense molecules useful, for example, in gene regulation of the polypeptides identified in Tables 1-6 or as antisense primers in amplification reactions of the nucleic acid sequences shown in Tables 1-6. Further, such sequences can be used as part of ribozyme and/or triple helix sequences or to design small interfering RNA molecules, also useful for gene regulation. Still further, such molecules can be used as components of diagnostic methods whereby, for example, the presence of a particular protein allele or alternatively-spliced protein transcript responsible for altering cellular responses mediated by NF-κB, or causing or predisposing one to an NF-κB-related disorder or condition can be detected.

Moreover, due to the degeneracy of the genetic code, other DNA sequences that encode substantially the amino acid sequences of the proteins identified in Tables 1-6 can be used in the practice of the present invention, e.g., for the cloning and expression of NF-κB pathway-associated polypeptides. Such DNA sequences include those that are capable of hybridizing to the nucleic acids identified in Tables 1-6 under stringent (high or moderate) conditions, or that would be capable of hybridizing under stringent conditions but for the degeneracy of the genetic code. Typically, the nucleic acids of the invention should exhibit at least about 80% overall sequence homology at the nucleotide level, more preferably at least about 85-90% overall homology and most preferably at least about 95% overall homology to the nucleic acid sequences of Tables 1-6 (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); (e.g., as determined by the CLUSTAL W algorithm using default parameters (J. D. Thompson et al., 1994, Nucleic Acids Research, 2(22):4673-4680).

Alternatively, the polypeptides of the invention should exhibit at least about 96%, 97%, 98%, 99%, 99.1%, 99.2%, 99.3%, 99.4%, 99.5%, 99.6%, 99.7%, 99.8%, or 99.9% overall homology to the amino acid sequence identified in Tables 1-6 (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 445, 447, 449, 451, 453, 455, 457, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 531, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 613, 615, 617, 619, 621, 623, 625, 627, 629, 631, 633, 635, 637, 640, 642, 644, 646, 648, 650, 652, 654, 656, 658, 660, 662, 664, 666, 668, 670, 672, 674, 676, 750, 752, 754, 756, 758, 760, 762, 764, 766, 768, 770, 772, 774, 776, 778 & 780) (e.g., as determined by the CLUSTAL W algorithm using default parameters (J. D. Thompson et al., 1994, Nucleic Acids Research, 2(22):4673-4680).

Those having skill in the art will know how to determine percent identity between/among sequences using, for example, algorithms such as those used in the GAP computer program (S. B. Needleman and C. D. Wunsch, 1970, “A general method applicable to the search for similarities in the amino acid sequence of two proteins”, J. Mol. Biol., 48(3):443-53) or based on the CLUSTALW computer program, mentioned above, or FASTDB, (Brutlag et al., 1990, Comp. App. Biosci., 6:237-245). Although the FASTDB algorithm typically does not consider internal non-matching deletions or additions in sequences, i.e., gaps, in its calculation, this can be corrected manually to avoid an overestimation of the % identity. GAP and CLUSTALW, however, do take sequence gaps into account in their identity calculations.

Also available to those having skill in this art are the BLAST and BLAST 2.0 algorithms (Altschul et al., 1977, Nuc. Acids Res., 25:3389-3402 and Altschul et al., 1990, J. Mol. Biol., 215:403-410). The BLASTN program for nucleic acid sequences uses as defaults a wordlength (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff & Henikoff, 1989, Proc. Natl. Acad. Sci., USA, 89:10915) uses alignments (B) of 50, expectation (E) of 10, M=5, N=4, and a comparison of both strands.

Altered nucleic acid sequences of the sequences shown in Tables 1-6 that can be used in accordance with the invention include deletions, additions or substitutions of different nucleotide residues resulting in a modified nucleic acid molecule, i.e., mutated or truncated, that encodes the same or a functionally equivalent gene product. The gene product itself may contain deletions, additions or substitutions of amino acid residues within the protein sequence of those identified in Tables 1-6, which result in a silent change, thus producing a functionally equivalent NF-κB pathway-associated polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine, arginine and histidine; amino acids with uncharged polar head groups having similar hydrophilicity values include the following: leucine, isoleucine, valine, glycine, alanine, asparagine, glutamine, serine, threonine, phenylalanine, tyrosine. A functionally equivalent polypeptide of those identified in Tables 1-6 can include a polypeptide which displays the same type of biological activity (e.g., regulation of NF-κB-mediated expression) as the native proteins, but not necessarily to the same extent.

The nucleic acid molecules or polynucleotide sequences of Tables 1-6 can be engineered in order to alter the coding sequences for a variety of reasons, including but not limited to, alterations that modify processing and expression of the gene products. For example, mutations may be introduced using techniques that are well known in the art, e.g., site-directed mutagenesis, to insert new restriction sites, to alter glycosylation patterns, phosphorylation, etc. For example, in certain expression systems such as yeast, host cells may over-glycosylate the gene product. When using such expression systems, it may be preferable to alter the protein coding sequences to eliminate any N-linked glycosylation sites.

In another embodiment, the nucleic acid sequences shown in Tables 1-6, including modified nucleic acids, can be ligated to a heterologous protein-encoding sequence to encode a fusion protein. Preferably, the nucleic acid is one that encodes a polypeptide with an activity of an NF-κB pathway-associated protein as described herein, or a portion or fragment thereof, and is linked, uninterrupted by stop codons and in frame, to a nucleotide sequence that encodes a heterologous protein or peptide. The fusion protein can be engineered to contain a cleavage site, located between the NF-κB pathway-associated protein sequence and the heterologous protein sequence, so that the NF-κB pathway-associated proteins can be cleaved away from the heterologous moiety. Nucleic acid sequences encoding fusion proteins can include full length NF-κB pathway-associated protein coding sequences, sequences encoding truncated proteins, sequences encoding mutated proteins, or sequences encoding peptide fragments of NF-κB pathway-associated proteins. The nucleic acid molecules of the invention can also be used as hybridization probes for obtaining cDNAs or genomic DNA. In addition, nucleic acids can be used as primers in PCR amplification methods to isolate NF-κB pathway-associated protein cDNAs and genomic DNA, e.g., from other species.

The sequences identified in Tables 1-6 can also be used to isolate NF-κB pathway-associated protein genes, including mutant or variant alleles. Such mutant or variant alleles can be isolated, for example, from individuals either known or proposed to have a genotype related to NF-κB-associated disorders, conditions, or dysfinctions. Mutant or variant alleles and mutant or variant allele gene products can then be used in the screening, therapeutic and diagnostic systems described herein. In addition, such NF-κB pathway-associated gene sequences can be used to detect genetic defects that can affect NF-κB-related disorders. For example, the present invention also encompasses naturally occurring polymorphisms of NF-κB pathway-associated protein genes including, but not limited to, single nucleotide polymorphisms (SNPs) in coding and noncoding regions.

In a further embodiment, the coding sequences of the proteins identified in Tables 1-6 can be synthesized in whole or in part, using chemical methods well known in the art, based on the nucleic acid and/or amino acid sequences of the NF-κB pathway-associated genes and proteins, respectively. (See, for example, Caruthers et al., 1980, Nuc. Acids Res. Symp. Ser., 7: 215-233; Crea and Horn, 1980, Nuc. Acids Res., 9(10): 2331; Matteucci and Caruthers, 1980, Tetrahedron Letters, 21: 719; and Chow and Kempe, 1981, Nuc. Acids Res., 9(12): 2807-2817). The invention encompasses (a) DNA vectors that contain any of the foregoing nucleic acids as shown in Tables 1-6 and/or their complements; (b) DNA expression vectors that contain any of the foregoing coding sequences as shown in operatively associated with a regulatory element that directs the expression of the coding sequences; and (c) genetically engineered host cells that contain any of the foregoing coding sequences as shown in Tables 1-6 operatively associated with a regulatory element that directs the expression of the coding sequences in the host cell. As used herein, regulatory elements include, but are not limited to, inducible and non-inducible promoters, enhancers, operators and other elements that drive and regulate expression, as known to those skilled in the art. Nonlimiting examples of such regulatory elements include the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention further relates to nucleic acid analogs, including but not limited to, peptide nucleic acid analogues, equivalent to the nucleic acid molecules described herein. “Equivalent” as used in this context refers to nucleic acid analogs that have the same primary base sequence as the nucleic acid molecules described above and shown in Tables 1-6. Nucleic acid analogs and methods for the synthesis of nucleic acid analogs are well known to those of skill in the art. (See, e.g., Egholm, M. et al., 1993, Nature, 365:566-568; and Perry-O'Keefe, H. et al., 1996, Proc. Natl. Acad. USA, 93:14670-14675).

### NF-κB Pathway-Associated Polypeptides and Peptides, and Expression Thereof

The nucleic acid sequences identified herein can be used to generate recombinant DNA molecules that direct the expression of the NF-κB pathway-associated protein (polypeptide) or peptides thereof in appropriate host cells, including the full-length proteins, functionally active or equivalent proteins and polypeptides, e.g., mutated, truncated or deleted forms of NF-κB pathway-associated proteins, peptide fragments, or fusion proteins. A functionally equivalent polypeptide can include a polypeptide that displays the same type of biological activity (e.g., regulation or modulation of second messenger activity and/or function) as the native protein, but not necessarily to the same extent. Such recombinantly expressed NF-κB pathway-associated molecules are useful in the various screening assays for determining modulators of NF-κB pathway-associated proteins, particularly for treatments and therapies of NF-κB-related disorders as described herein.

In a specific embodiment, the amino acid sequence of the newly identified NF-κB pathway-associated polypeptides are identified in Tables 1-6. Both the NF-κB pathway-associated polypeptide and peptide sequences are useful as targets, and/or as immunogens to generate antibodies for the methods and compositions according to the present invention. The proteins and polypeptides of the invention include peptide fragments of NF-κB pathway-associated proteins, peptides corresponding to one or more domains of the protein, mutated, truncated or deleted forms of the proteins and polypeptides, as well as fision proteins; all of the aforementioned NF-κB pathway-associated protein derivatives can be obtained by techniques well known in the art, given the nucleic acid and amino acid sequences as described herein. The proteins and corresponding peptides can also contain deletions, additions or substitutions of amino acid residues within the protein sequence, which can result in a silent change, thus producing a functionally equivalent NF-κB pathway-associated polypeptide. Such amino acid substitutions can be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine, arginine and histidine; amino acids with uncharged polar head groups having similar hydrophilicity values include the following: leucine, isoleucine, valine, glycine, alanine, asparagine, glutamine, serine, threonine, phenylalanine, tyrosine.

The NF-κB pathway-associated polypeptides should exhibit at least about 80% overall sequence identity at the amino acid level, more preferably at least about 85-90% overall identity and most preferably at least about 95% overall identity to the amino acid sequence of Tables 1-6 (e.g., as determined by the CLUSTAL W algorithm using default parameters (J. D. Thompson et al., 1994, Nucleic Acids Research, 2(22):4673-4680).

Alternatively, the NF-κB pathway-associated polypeptide should exhibit at least about 96%, 97%, 98%, 99%, 99.1%, 99.2%, 99.3%, 99.4%, 99.5%, 99.6%, 99.7%, 99.8%, or 99.9% overall identity to the NF-κB pathway-associated amino acid sequence as depicted in Tables 1-6 (e.g., as determined by the CLUSTAL W algorithm using default parameters (J. D. Thompson et al., 1994, Nucleic Acids Research, 2(22):4673-4680).

Mutated or altered forms of the NF-κB pathway-associated proteins and peptides can be obtained using random mutagenesis techniques, site-directed mutagenesis techniques, or by chemical methods, e.g., protein synthesis techniques, as practiced in the art. Mutant NF-κB pathway-associated proteins or peptides can be engineered so that regions important for function are maintained, while variable residues are altered, e.g., by deletion or insertion of an amino acid residue(s) or by the substitution of one or more different amino acid residues. For example, conservative alterations at the variable positions of a polypeptide can be engineered to produce a mutant polypeptide that retains the function of a NF-κB pathway-associated protein. Non-conservative alterations of variable regions can be engineered to alter NF-κB pathway-associated protein function, if desired. Alternatively, in those cases where modification of function (either to increase or decrease function) is desired, deletion or non-conservative alterations of conserved regions of the NF-κB pathway-associated polypeptides can be engineered.

In another aspect, fuision proteins containing NF-κB pathway-associated amino acid sequences can also be obtained by techniques known in the art, including genetic engineering and chemical protein synthesis techniques. According to this aspect, NF-κB pathway-associated fusion proteins are encoded by an isolated nucleic acid molecule comprising a nucleic acid that encodes a polypeptide with an activity of a NF-κB pathway-associated protein, or a fragment thereof, linked in frame and uninterrupted by stop codons, to a nucleotide sequence that encodes a heterologous protein or peptide.

Fusion proteins include those that contain the full-length NF-κB pathway-associated amino acid sequences, peptide sequences, e.g., encoding one or more functional domains, mutant amino acid sequences, or truncated amino acid sequences linked to an unrelated protein or polypeptide sequence. Such fuision proteins include, but are not limited to, Ig Fc fusions which stabilize the NF-κB pathway-associated fusion protein and can prolong the half-life of the protein in vivo, or fusions to an enzyme, fluorescent protein or luminescent (chemiluminescent) protein that provides a marker function.

NF-κB pathway-associated polypeptides, proteins, peptides, and derivatives thereof, can be produced using genetic engineering techniques. Thus, in order to express a biologically active NF-κB pathway-associated polypeptide by recombinant technology, a nucleic acid molecule coding for the polypeptide, or a functional equivalent thereof, is inserted into an appropriate expression vector, i.e., a vector which contains the necessary elements for the transcription and translation of the inserted coding sequence. More specifically, the NF-κB pathway-associated nucleic acids are operatively associated with regulatory nucleotide sequences containing transcriptional and/or translational regulatory information that controls expression of the NF-κB pathway-associated nucleic acids in the host cell. The NF-κB pathway-associated gene products so produced, as well as host cells, or cell lines transfected or transformed with recombinant expression vectors, can be used for a variety of purposes. These include, but are not limited to, generating antibodies (i.e., monoclonal or polyclonal) that bind to the NF-κB pathway-associated proteins or peptides, including those that competitively inhibit binding and thus “neutralize” NF-κB pathway-associated protein activity, and the screening and selection of NF-κB pathway-associated protein analogs, ligands, or interacting molecules.

In instances in which the NF-κB pathway-associated coding sequence is engineered to encode a cleavable fusion protein, purification can be readily accomplished using affinity purification techniques. For example, a collagenase cleavage recognition consensus sequence can be engineered between the carboxy terminus of NF-κB pathway-associated protein and protein A. The resulting fusion protein can be purified using an IgG column that binds to the protein A moiety. Unfused NF-κB pathway-associated protein can be released from the column by treatment with collagenase. Another example embraces the use of pGEX vectors that express foreign polypeptides as fusion proteins with glutathione S-transferase (GST). The fusion protein can be engineered with either thrombin or factor Xa cleavage sites between the cloned gene and the GST moiety. The fusion protein can be easily purified from cell extracts by adsorption to glutathione agarose beads, followed by elution in the presence of glutathione. In fact, any cleavage site or enzyme cleavage substrate can be engineered between the NF-κB pathway-associated gene product sequence and a second peptide or protein that has a binding partner which can be used for purification, e.g., any antigen for which an immunoaffinity column can be prepared.

In preferred embodiments, for example, cell lines transfected with NF-κB pathway-associated polypeptides are useful for the identification of agonists and antagonists of the polypeptides. Representative uses of these cell lines include employing the cell lines in a method of identifying NF-κB pathway-associated protein agonists and antagonists. Preferably, the cell lines are useful in a method for identifying a compound that modulates the biological activity of the polypeptides, comprising the steps of: (a) combining a candidate modulator compound with a host cell expressing a NF-κB pathway-associated polypeptide having the sequence as set forth in Tables 1-6; and (b) measuring an effect of the candidate modulator compound on the activity of the expressed polypeptide.

In addition, NF-κB pathway-associated fusion proteins can be readily purified by utilizing an antibody specific for the fusion protein being expressed. For example, a system described by Janknecht et al. allows for the purification of non-denatured fusion proteins expressed in human cell lines (Janknecht, et al., 1991, Proc. Natl. Acad. Sci. USA, 88: 8972-8976). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

Alternatively, NF-κB pathway-associated proteins and peptides can be produced using chemical methods to synthesize the NF-κB pathway-associated amino acid sequences in whole or in part. For example, peptides can be synthesized by solid phase techniques, cleaved from the resin, and purified by preparative high performance liquid chromatography (see, e.g., Creighton, 1983, Proteins: Structures And Molecular Principles, W.H. Freeman and Co., New York., pp. 50-60). The composition of the synthetic peptides can be confirmed by amino acid analysis or sequencing (e.g., the Edman degradation procedure; see Creighton, 1983, Proteins: Structures and Molecular Principles, W.H. Freeman and Co., New York., pp. 34-49).

The NF-κB pathway-associated proteins, polypeptides and peptide fragments, mutated, truncated or deleted forms of NF-κB pathway-associated proteins and/or NF-κB pathway-associated fusion products can be prepared for various uses, including but not limited to, the generation of antibodies, as reagents in diagnostic assays, the identification of other cellular gene products associated with NF-κB pathway-associated in the development or continuance of NF-κB pathway-related disorders, and as reagents in assays for screening for compounds for use in the treatment of NF-κB-related diseases and disorders.

In a particular related embodiment, NF-κB pathway-associated peptides, derived from the sequences shown in Tables 1-6, can be used to identify individuals who are at risk for developing NF-κB-related disorders or the underlying symptoms thereof. Such identification can be achieved by a variety of diagnostic or screening methods and assays as are known in the art. For example, antibodies specific for the NF-κB pathway-associated polypeptides or peptides identified in Tables 1-6 can be used in such assays, in addition to primers directed against the polynucleotide sequence that codes for the proteins or peptide. Primers are preferably obtained from the nucleic acid sequences encoding the NF-κB pathway-associated polypeptides of Tables 1-6 (see, for instance, primers shown in Example 2).

### Vectors and Host Cells

A variety of host-expression vector systems can be used to express the NF-κB pathway-associated polypeptide coding sequences. Such host-expression systems represent vehicles by which the coding sequences of interest can be produced and subsequently purified, but also represent cells which can, when transformed or transfected with the appropriate nucleotide coding sequences, exhibit the corresponding NF-κB pathway-associated gene product(s) in situ and/or function in vivo. These hosts include, but are not limited to, microorganisms such as bacteria (e.g., E. coli, B. subtilis) transformed with recombinant bacteriophage DNA, plasmid DNA, or cosmid DNA expression vectors containing the NF-κB pathway-associated coding sequences; yeast (e.g., Saccharomyces, Pichia) transformed with recombinant yeast expression vectors containing the NF-κB pathway-associated coding sequences; insect cell systems infected with recombinant virus expression vectors (e.g., baculovirus) containing the NF-κB pathway-associated coding sequences; plant cell systems infected with recombinant virus expression vectors (e.g., cauliflower mosaic virus (CaMV); tobacco mosaic virus (TMV)) or transformed with recombinant plasmid expression vectors (e.g., Ti plasmid) containing the NF-κB pathway-associated coding sequences; or mammalian cell systems, including human cells, (e.g., COS, CHO, BHK, 293, NIH/3T3) harboring recombinant expression constructs containing promoters derived from the genome of mammalian cells as described below.

The expression elements of these systems can vary in their strength and specificities. Depending on the host/vector system utilized, any of a number of suitable transcriptional and translational elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used; when cloning in insect cell systems, promoters such as the baculovirus polyhedrin promoter can be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) can be used; when cloning in mammalian cell systems, promoters derived from the genome of mammalian cells (e.g., metallothionein promoter) or from mammalian viruses (e.g., the adenovirus late promoter; the vaccinia virus 7.5K promoter) can be used; when generating cell lines that contain multiple copies of NF-κB pathway-associated protein DNA, SV40-, BPV- and EBV-based vectors can be used with an appropriate selectable marker.

In bacterial systems, a number of expression vectors can be advantageously selected depending upon the use intended for the expressed NF-κB pathway-associated polypeptides or peptides. For example, when large quantities of the NF-κB pathway-associated polypeptides or peptides are to be produced, e.g., for the generation of antibodies or for the production of the NF-κB pathway-associated gene products, vectors that direct the expression of high levels of fusion protein products that are readily purified may be desirable. Such vectors include, but are not limited to, the E. coli expression vector pUR278 (Ruther et al., 1983, EMBO J., 2:1791), in which the NF-κB pathway-associated protein coding sequence can be ligated into the vector in-frame with the lacZ coding region so that a hybrid NF-κB pathway-associated protein/lacZ protein is produced; pIN vectors (Inouye & Inouye, 1985, Nucleic Acids Res., 13: 3101-3109; Van Heeke & Schuster, 1989, J. Biol. Chem., 264: 5503-5509); and the like. pGEX vectors can also be used to express foreign polypeptides as fusion proteins with glutathione S-transferase (GST). In general, such fusion proteins are soluble and can easily be purified from lysed cells by affinity chromatography, e.g., adsorption to glutathione-agarose beads followed by elution in the presence of free glutathione. The pGEX vectors are designed to include thrombin or factor Xa protease cleavage sites so that the cloned polypeptide of interest can be released from the GST moiety. See also Booth et al., 1988, Immunol. Lett., 19: 65-70; and Gardella et al., 1990, J. Biol. Chem., 265: 15854-15859; Pritchett et al., 1989, Biotechniques, 7: 580.

In yeast, a number of vectors containing constitutive or inducible promoters are suitable for use. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, In: Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Cold Spring Harbor Press, Vols. I and II.

In an insect system, Autographa californica nuclear polyhedrosis virus (AcNPV) can be used as a vector to express foreign genes. The virus grows in Spodoptera frugiperda cells. The NF-κB pathway-associated protein encoding sequences can be cloned into non-essential regions (for example, the polyhedrin gene) of the virus and placed under the control of an AcNPV promoter (for example, the polyhedrin promoter). Successful insertion of the NF-κB pathway-associated coding sequence results in inactivation of the polyhedrin gene and production of non-occluded recombinant virus (i.e., virus lacking the proteinaceous coat coded for by the polyhedrin gene). These recombinant viruses can then be used to infect Spodoptera frugiperda cells in which the inserted gene is expressed (see e.g., Smith et al., 1983, J. Virol., 46: 584; Smith, U.S. Pat. No. 4,215,051).

In mammalian host cells, a number of virus-based expression systems can be employed. In cases where an adenovirus is used as an expression vector, the NF-κB pathway-associated protein coding sequence can be ligated to an adenovirus transcription/translation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) results in a recombinant virus that is viable and capable of expressing NF-κB pathway-associated protein in infected hosts (see, e.g., Logan & Shenk, 1984, Proc. Natl. Acad. Sci. USA, 81: 3655-3659). Alternatively, the vaccinia 7.5K promoter can be used (see, e.g., Mackett et al., 1982, Proc. Natl. Acad. Sci. USA, 79: 7415-7419; Mackett et al., 1984, J. Virol., 49: 857-864; Panicali et al., 1982, Proc. Natl. Acad. Sci. USA, 79: 4927-4931).

Specific initiation signals may also be required for efficient translation of inserted NF-κB pathway-associated coding sequences. These signals include the ATG initiation codon and adjacent sequences. In cases where an entire NF-κB pathway-associated gene, including its own initiation codon and adjacent sequences, is inserted into the appropriate expression vector, no additional translational control signals may be needed. However, in cases where only a portion of the coding sequence is inserted, exogenous translational control signals, including the ATG initiation codon, are preferably provided. Furthermore, the initiation codon is preferably in phase with the reading frame of the coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol., 153:516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can frequently be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include, but are not limited to, CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell lines such as, for example, CRL7030 and Hs578Bst, and the like.

For long-term, high-yield production of recombinant proteins, stable expression is preferred. For example, cell lines that stably express the NF-κB pathway-associated polypeptides or peptides are engineered. Thus, rather than using expression vectors which contain viral origins of replication, host cells can be transformed with NF-κB pathway-associated protein encoding nucleic acid molecules, e.g., DNA, controlled by appropriate expression control elements (e.g., promoter, enhancer, sequences, transcription terminators, polyadenylation sites, etc.), and at least one selectable marker. Following the introduction of the foreign DNA, engineered cells are allowed to grow for about 1-2 days in an enriched medium, and then are placed in selective medium. The selectable marker in the recombinant plasmid confers resistance to the selection medium and allows cells to stably integrate the plasmid into their chromosomes and grow to form foci that, in turn, can be cloned and expanded into cell lines. This method can advantageously be used to engineer cell lines that express cellular NF-κB pathway-associated polypeptides or peptides. Such engineered cell lines are particularly useful in screening for NF-κB pathway-associated protein analogs or ligands, or for determining compounds, molecules, and the like, which modulate NF-κB pathway-associated protein expression or function.

In instances in which the mammalian cell is a human cell, human artificial chromosome (HAC) systems are among the expression systems by which NF-κB pathway-associated nucleic acid sequences can be expressed (see, e.g., Harrington et al., 1997, Nature Genetics, 15: 345-355).

Host cells which contain the NF-κB pathway-associated protein coding sequences and which preferably express a biologically active gene product can be identified by at least four general approaches: (a) DNA-DNA or DNA-RNA hybridization; (b) the presence or absence of “marker” gene functions; (c) assessing the level of transcription as measured by the expression of NF-κB pathway-associated protein mRNA transcripts in the host cell; and (d) detection of the gene product as measured by immunoassay or by its biological activity.

In the first approach, the presence of the NF-κB pathway-associated protein coding sequences inserted into the expression vector can be detected by DNA-DNA or DNA-RNA hybridization using probes comprising nucleotide sequences that are homologous to the coding sequences, respectively, or portions or derivatives thereof.

In the second approach, the recombinant expression vector/host system can be identified and selected based upon the presence or absence of certain “marker” gene functions. For example, if the NF-κB pathway-associated coding sequences are inserted within a marker gene sequence of the vector, recombinants containing the coding sequence can be identified by the absence of the marker gene function. Alternatively, a marker gene can be placed in tandem with the NF-κB pathway-associated protein sequence under the control of the same or a different promoter used to control the expression of the coding sequence. Expression of the marker in response to induction or selection indicates expression of the NF-κB pathway-associated coding sequence.

Selectable markers include, for example, resistance to antibiotics, resistance to methotrexate, transformation phenotype, and occlusion body formation in baculovirus. In addition, thymidine kinase activity (M. Wigler et al., 1977, Cell, 11: 223) hypoxanthine-guanine phosphoribosyltransferase (Szybalska & Szybalski, 1962, Proc. Natl. Acad. Sci. USA, 48: 2026), and adenine phosphoribosyltransferase (Lowy et al., 1980, Cell, 22: 817) genes can be employed in tk⁻, hgprt⁻ or aprt⁻ cells, respectively. Also, anti-metabolite resistance can be used as the basis of selection for dhfr, which confers resistance to methotrexate (M. Wigler et al., 1980, Proc. Natl. Acad. Sci. USA, 77: 3567; O'Hare et al., 1981, Proc. Natl. Acad. Sci. USA, 78: 1527); gpt, which confers resistance to mycophenolic acid (Mulligan & Berg, 1981, Proc. Natl. Acad. Sci. USA, 78: 2072); neo, which confers resistance to the aminoglycoside G-418 (Colberre-Garapin, et al., 1981, J. Mol. Biol., 150: 1); and hygro, which confers resistance to hygromycin (Santerre et al., 1984, Gene, 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA, 85: 8047); and ODC (ornithine decarboxylase) which confers resistance to the ornithine decarboxylase inhibitor, 2-(difluoromethyl)-DL-ornithine, DFMO (McConlogue, 1987, in Current Communications in Molecular Biology, Cold Spring Harbor Laboratory ed.).

In the third approach, transcriptional activity for the NF-κB pathway-associated protein coding region can be assessed by hybridization assays. For example, RNA can be isolated and analyzed by Northern blot using a probe homologous to the NF-κB pathway-associated protein coding sequence or particular portions thereof Alternatively, total nucleic acids of the host cell can be extracted and assayed for hybridization to such probes.

In the fourth approach, the expression of the NF-κB pathway-associated proteins or peptide products can be assessed immunologically, for example by Western blots, immunoassays such as radio-immunoprecipitation, enzyme-linked immunoassays and the like. The ultimate test of the success of the expression system, however, involves the detection of biologically active NF-κB pathway-associated gene products. A number of assays can be used to detect NF-κB pathway-associated activity, including but not limited to, binding assays and biological assays for NF-κB pathway-associated activity.

Once a cell clone that produces high levels of a biologically active NF-κB pathway-associated polypeptide is identified, the cloned cells can be expanded and used to produce large amounts of the polypeptide which can be purified using techniques well known in the art, including but not limited to, immunoaffinity purification using antibodies, immunoprecipitation, or chromatographic methods including high performance liquid chromatography (HPLC).

Cell lines expressing NF-κB pathway-associated proteins are also useful in a method of screening for a compound that is capable of modulating the biological activity of NF-κB pathway-associated polypeptides, comprising the steps of: (a) determining the biological activity of the polypeptide in the absence of a modulator compound; (b) contacting a host cell expressing the polypeptide with the modulator compound; and (c) determining the biological activity of the polypeptide in the presence of the modulator compound; wherein a difference between the activity of the polypeptide in the presence of the modulator compound and in the absence of the modulator compound indicates a modulating effect of the compound. Additional uses for such cell lines expressing NF-κB pathway-associated proteins are described herein or otherwise known in the art.

Methods that are well known to those skilled in the art are used to construct expression vectors containing the NF-κB pathway-associated protein or peptide coding sequences and appropriate transcriptional and translational control elements and/or signals. These methods include in vitro recombinant DNA techniques, synthetic techniques and in vivo recombination/genetic recombination. See, for example, the techniques described in Maniatis et al., 1989, Molecular Cloning, A Laboratory Manual, Cold Spring Harbor Laboratory, N.Y. and Ausubel et al., 1989, Current Protocols in Molecular Biology, Greene Publishing Associates and Wiley Interscience, N.Y. See also, Sambrook et al., 1989, Molecular Cloning, A Laboratory Manual, Cold Spring Harbor Press, N.Y.

Modulation of NF-κB Pathway-Associated Polypeptides: Methods, Compounds and Compositions Related Thereto

In another embodiment, modulators of NF-κB pathway-associated proteins are particularly embraced by the present invention. Modulators can include any molecule, e.g., protein, peptide, oligopeptide, small organic molecule, chemical compound, polysaccharide, polynucleotide, etc., having the capability to directly or indirectly alter or modify the activity or function of the NF-κB pathway-associated polypeptide. In a specific embodiment, this invention encompasses modulators of the proteins identified in Tables 1-6. Candidate modulatory agents or compounds or materials can encompass numerous chemical classes, though typically they are organic molecules, preferably small organic compounds, for example, without limitation, those having a molecular weight of more than 100 and less than about 10,000 daltons, preferably, less than about 2000 to 5000 daltons. Candidate modulatory compounds can comprise functional groups necessary for structural interaction with proteins, particularly hydrogen bonding, and typically include at least an amine, carbonyl, hydroxyl or carboxyl group, preferably at least two of the functional chemical groups. The candidate compounds often comprise cyclical carbon or heterocyclic structures and/or aromatic or polyaromatic structures substituted with one or more of the above functional groups. Candidate compounds are also found among biomolecules including peptides, saccharides, fatty acids, steroids, purines, pyrimidines, derivatives, structural analogs or combinations thereof.

Modulatory agents or compounds can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fingal, plant and animal extracts are available or readily produced. In addition, natural or synthetically produced libraries and compounds are readily modified through conventional chemical, physical and biochemical means. Known pharmacological agents can also be subjected to directed or random chemical modifications, such as acylation, alkylation, esterification, amidification to produce structural analogs.

Modulators of the NF-κB pathway-associated proteins as embraced by this invention can be antagonists, suppressors, inhibitors, or blockers of the proteins, as such modulators can be efficacious in affecting NF-κB-mediated events or reducing the symptoms underlying NF-κB-related disorders. An antagonist is typically a molecule which, when bound to, or associated with, a NF-κB pathway-associated polypeptide, or a functional fragment thereof, decreases or inhibits the amount or duration of the biological or immunological activity of the polypeptide. Antagonists can include proteins, nucleic acids, carbohydrates, antibodies, or any other molecules that decrease or reduce the effect of a NF-κB pathway-associated polypeptide. Antagonists typically, diminish, inhibit, block, decrease, reduce, suppress, or abolish the function or activity of an NF-κB pathway-associated molecule. More specifically, modulators of NF-κB pathway-associated proteins can be efficacious in treating, ameliorating, or preventing NF-κB-related conditions or disease including, but not limited to, those disclosed herein.

In addition, modulators such as agonists or enhancers of NF-κB pathway-associated protein function or activity are embraced by the present invention, particularly, for a NF-κB pathway-associated protein target that is part of a reparative, reversing, and/or protective mechanism, which is induced following the exposure of cells to harmful or deleterious extracellular signals. Agonists typically are molecules which, when bound to, or associated with, a NF-κB pathway-associated polypeptide, or a functional fragment thereof, increase, enhance, or prolong the duration of the effect of the NF-κB pathway-associated polypeptide. Agonists may include proteins, peptides, nucleic acids, carbohydrates, or any other molecules that bind to and modulate the effect of the NF-κB pathway-associated polypeptide. Agonists typically enhance, increase, or augment the function or activity of an NF-κB pathway-associated molecule. As such, an agonist compound may be efficacious in enhancing the protective mechanism of a NF-κB pathway-associated protein in alleviating the symptoms of NF-κB-related diseases.

### Screening Assays for Determining Compounds that Modulate NF-κB Pathway-Associated Polypeptides, the NF-κB pathway, and Components Thereof and Compositions Related Thereto

Screening assays can be used to identify compounds that modulate NF-κB pathway-associated polypeptide function or activity, the NF-κB pathway, or components thereof. Such compounds can include, but are not limited to, peptides, small organic or inorganic molecules or macromolecules such as nucleic acid molecules or proteins, e.g., antibodies and antibody fragments, and can be utilized, for example, in the control and/or treatment of NF-κB related disorders, in the modulation of second messenger or cellular molecules which are regulated or modulated by NF-κB pathway-associated polypeptides and which affect the NF-κB pathway and its related conditions and disorders. These compounds may also be useful, e.g., in elaborating the biological functions of the NF-κB pathway-associated gene products, i.e., the NF-κB pathway-associated proteins and their peptides, in modulating the proteins biological functions and for preventing, treating, reducing, and/or ameliorating symptoms and/or physiological characteristics and effects of NF-κB pathway-related disorders.

The compositions of the invention include pharmaceutical compositions comprising one or more of the NF-κB pathway-associated polypeptide modulator compounds. Such pharmaceutical compositions can be formulated as discussed hereinbelow. More specifically, these compounds can include compounds that bind to NF-κB pathway-associated polypeptides and peptide components, compounds that bind to other proteins or molecules that interact with the NF-κB pathway-associated gene products and/or interfere with the interaction of the NF-κB pathway-associated gene products with other proteins or molecules, and compounds that modulate the activity of the genes, i.e., modulate the level of NF-κB pathway-associated polypeptide gene expression and/or modulate the level of the gene product or protein activity.

In a related aspect, assays can be utilized that identify compounds that bind to gene regulatory sequences, e.g., promoter sequences (see e.g., K. A. Platt, 1994, J. Biol. Chem., 269:28558-28562); such compounds may modulate the level of NF-κB pathway-associated polypeptide gene expression. In addition, functional assays can be used to screen for compounds that modulate NF-κB pathway-associated gene product activity. In such assays, compounds are screened for agonistic or antagonistic activity with respect to the biological activity or function of the NF-κB pathway-associated proteins, polypeptides, or peptides, such as changes in the intracellular levels or activity of a molecule with which the NF-κB pathway-associated polypeptide interacts or which is regulated by the NF-κB pathway-associated polypeptide, changes in regulatory factor release, or other activities or functions of the NF-κB pathway-associated protein, polypeptide or peptides which are involved in causing or maintaining NF-κB pathway-related disorders according to this invention.

According to an embodiment of this invention, molecules that are affected, regulated, modulated, or that otherwise interact with NF-κB pathway-associated polypeptides, for example, molecules of the NF-κB pathway, can be monitored or assayed in polypeptide-expressing host cells to determine if modulators of the polypeptides (e.g., antagonists such as antisense of the polypeptide as described further herein) affect the function of component molecules in the pathway. In a particular aspect of this embodiment, antisense molecules to NF-κB pathway-associated sequences were used to evaluate the outcome of NF-κB-mediated gene expression (Example 5).

The ability of NF-κB pathway-associated polypeptides to regulate NF-κB functions in NF-κB pathway-associated polypeptide expressing cells supports the view that antagonist and agonists to the NF-κB pathway-associated polypeptides would have an impact on many diseases, including autoimmune diseases, inflammation, asthma, COPD, rheumatoid arthritis (RA), cancers, such as, but not limited to, lung cancer, stomach cancer, breast cancer, testicular cancer, ovarian cancer, cervical cancer, genitourinary tract cancer, bladder cancer, prostate cancer, gastrointestinal cancer, colon cancer, esophageal cancer, head and neck cancer, cancer of the brain, thyroid cancer, liver cancer, pancreatic cancer, kidney cancer, etc., ischemia-reperfusion injury, atherosclerosis, thrombosis, other vascular diseases and HIV.

According to another embodiment of this invention, screening assays can be designed to identify compounds capable of binding to the NF-κB pathway-associated gene product or peptides thereof. Such compounds can be useful, e.g., in modulating the activity of wild type and/or mutant gene products, in elaborating the biological function of the gene product, and in screens for identifying compounds that disrupt normal gene product interactions. Alternatively, such compounds may in themselves disrupt such interactions.

Screening assays to identify compounds that bind to NF-κB pathway-associated polypeptides, and/or their composite peptides can involve preparing a reaction mixture of the polypeptide or peptide and a test compound under conditions and for a time sufficient to allow the two components to interact with, i.e., bind to each other, and thus form a complex, which can represent a transient complex that can be removed and/or detected in the reaction mixture. For example, one type of assay involves anchoring a NF-κB pathway-associated polypeptide or peptide, or the test substance, onto a solid phase and detecting the polypeptide or peptide/test compound complexes anchored on the solid phase at the end of the reaction. In one aspect of such a method, the NF-κB pathway-associated polypeptide or peptide can be anchored onto a solid surface, and the test compound, which is not anchored, can be labeled, either directly or indirectly.

The detection of complexes anchored on the solid surface can be accomplished in a number of ways. In cases in which the previously non-immobilized component is pre-labeled, the detection of label immobilized on the surface indicates that complexes were formed. In cases in which the previously non-immobilized component is not pre-labeled, an indirect label can be used to detect complexes anchored on the surface; e.g., using a labeled antibody specific for the previously non-immobilized component (the antibody, in turn, can be directly labeled, or indirectly labeled with a labeled anti-Ig antibody).

Alternatively, a reaction can be conducted in a liquid phase, the reaction products separated from unreacted components, and complexes detected, e.g., using an immobilized antibody specific for the NF-κB pathway-associated polypeptide or peptide, or the test compound, to anchor any complexes formed in solution, and a labeled antibody specific for the other component of the formed complex to detect anchored complexes.

Compounds that modulate NF-κB pathway-associated protein activity can also include compounds that bind to proteins that interact with NF-κB pathway-associated polypeptides. These modulatory compounds can be identified by first identifying those proteins, e.g., cellular proteins, that interact with the NF-κB pathway-associated protein products, e.g., by standard techniques known in the art for detecting protein-protein interactions, such as co-immunoprecipitation, cross-linking and co-purification through gradients or chromatographic columns. Utilizing procedures such as these allows for the isolation of proteins that interact with the NF-κB pathway-associated polypeptides, peptides, or proteins.

Once isolated, such a protein can be identified and can, in turn, be used, in conjunction with standard techniques, to identify additional proteins with which that protein (and/or the NF-κB pathway-associated protein) interacts. For example, at least a portion of the amino acid sequence of the protein that interacts with a NF-κB pathway-associated gene product can be ascertained using techniques well known to those of skill in the art, such as via the Edman degradation technique (see, e.g., Creighton, 1983, Proteins: Structures and Molecular Principles, W.H. Freeman & Co., N.Y., pp.34-49). The amino acid sequence thus obtained can be used as a guide for the generation of oligonucleotide mixtures that can, in turn, be used to screen for gene sequences encoding the interacting proteins. Screening is accomplished, for example, by standard hybridization or PCR techniques. Techniques for the generation of oligonucleotide mixtures and screening are well-known and practiced in the art (see, e.g., F. M. Ausubel, supra, and PCR Protocols: A Guide to Methods and Applications, 1990, M. Innis et al., eds. Academic Press, Inc., New York).

In addition, methods can be employed that result in the simultaneous identification of genes which encode proteins that interact with the NF-κB pathway-associated polypeptides. These methods include, for example, probing expression libraries with labeled NF-κB pathway-associated polypeptide, using the polypeptide in a manner similar to the well-known technique of antibody probing of λgt11 libraries. One method that detects protein interactions in vivo is the two-hybrid system. A version of this system is described by Chien et al., 1991, Proc. Natl. Acad. Sci. USA, 88:9578-9582 and is commercially available from Clontech (Palo Alto, Calif.).

Compounds that disrupt the interaction of NF-κB pathway-associated polypeptides with other molecules, or binding partners, as determined by techniques exemplified above, can be useful in regulating the activity of the polypeptides, including mutant polypeptides. Such compounds can include, but are not limited to, molecules such as peptides, and the like, which bind to NF-κB pathway-associated polypeptides as described above. Illustrative assay systems used to identify compounds that interfere with the interaction between NF-κB pathway-associated polypeptides and their interacting molecule(s) involve preparing a reaction mixture containing a NF-κB pathway-associated polypeptide or peptide and the interacting molecule, under conditions and for a time sufficient to allow the two to interact (and bind), thus forming a complex. In order to test a compound for inhibitory activity, the reaction mixture is prepared in the presence and absence of the test compound. The test compound can be initially included in the reaction mixture, or it can be added at a time subsequent to the addition of the NF-κB pathway-associated polypeptide and its interacting molecule. Control reaction mixtures are incubated without the test compound or with a placebo. Complexes formed between the NF-κB pathway-associated polypeptides and the interacting molecule(s) are then detected. The formation of a complex in the control reaction, but not in the reaction mixture containing the test compound, indicates that the compound interferes with the interaction of the polypeptide and the interacting molecule. Further, complex formation within reaction mixtures containing the test compound and a normal NF-κB pathway-associated protein or peptide product can also be compared with complex formation within reaction mixtures containing the test compound and a mutant NF-κB pathway-associated protein or peptide product. This comparison could be particularly useful in those cases in which it is desirable to identify compounds that disrupt interactions of mutant but not normal NF-κB pathway-associated proteins.

Assaying for compounds that interfere with the interaction of the NF-κB pathway-associated proteins or peptides and interacting (e.g., modulated or regulated) molecules can be conducted in a heterogeneous or homogeneous format. Heterogeneous assays involve anchoring either the NF-κB pathway-associated polypeptide or the binding molecule onto a solid phase and detecting complexes anchored on the solid phase at the end of the reaction. In homogeneous assays, the entire reaction is carried out in a liquid phase. In either approach, the order of addition of the reaction components can be varied to obtain different information about the compounds being tested. For example, test compounds that interfere with the interaction between the NF-κB pathway-associated polypeptide and its interacting molecules, e.g., by competition, can be identified by conducting the reaction in the presence of the test substance; i.e., by adding the test substance to the reaction mixture prior to, or simultaneously with, the polypeptide and the interacting molecule. Alternatively, test compounds that disrupt preformed complexes, e.g., compounds with higher binding constants that displace one of the components from the complex, can be tested by adding the test compound to the reaction mixture after the complexes between NF-κB pathway-associated protein and another molecule or molecules have been formed. The various formats are described briefly below.

In a heterogeneous assay system, either the NF-κB pathway-associated polypeptide or the interacting molecule, is anchored onto a solid surface, while the non-anchored molecule is labeled, either directly or indirectly. In practice, microtiter plates are conveniently utilized. The anchored species can be immobilized by non-covalent or covalent attachments. Non-covalent attachment can be achieved simply by coating the solid surface with a solution comprising the polypeptide or the interacting molecule and drying the surface. Alternatively, an immobilized antibody specific for the molecule to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed remain immobilized on the solid surface. The detection of complexes anchored on the solid surface is performed in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of adding the reaction components, test compounds which inhibit complex formation, or which disrupt preformed complexes, can be detected.

Alternatively, the reaction can be conducted in a liquid phase in the presence or absence of the test compound, the reaction products separated from unreacted components, and complexes detected; e.g., using an immobilized antibody specific for one of the interacting components, to anchor any complexes formed in solution, and a labeled antibody specific for the other partner to detect anchored complexes. Again, depending upon the order of addition of reaction components to the liquid phase, test compounds that inhibit complex formation or that disrupt preformed complexes can be identified.

In another aspect of such assays, a preformed complex of the NF-κB pathway-associated polypeptide or peptide and an interacting molecule is prepared in which either the polypeptide or its interacting partner molecule is labeled. However, the signal generated by the label is quenched due to complex formation between the polypeptide and the interacting molecule (see, e.g., U.S. Pat. No. 4,109,496 to Rubenstein which utilizes this approach for immunoassays). The addition of a test substance that competes with and displaces one of the species from the preformed complex will result in the generation of a signal above background. In this way, test substances that disrupt NF-κB pathway-associated protein/interacting partner interactions can be identified.

Techniques as described above can be employed using the NF-κB pathway-associated peptide fragments that correspond to the binding domains of the NF-κB pathway-associated protein and/or the interacting partner, instead of one or both of the full-length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interacting, e.g., binding. Alternatively, one protein can be anchored to a solid surface using methods as described above, and allowed to interact with, e.g., bind, to its labeled interacting partner, which has been treated with a proteolytic enzyme, such as trypsin. After washing, a short, labeled peptide comprising the interacting, e.g., binding, domain may remain associated with the solid material; the associated domain can be isolated and identified by amino acid sequencing. Also, once the gene coding for the intracellular binding partner is obtained, short gene segments can be engineered to express peptide fragments of the protein, which can then be tested for binding activity and purified or synthesized.

The human NF-κB pathway-associated polypeptides and/or peptides, or immunogenic fragments or oligopeptides thereof, can be used for screening for therapeutic drugs or compounds for NF-κB pathway-related disorders in a variety of drug screening techniques. The fragment employed in such a screening assay can be free in solution, affixed to a solid support, borne on a cell surface, or located intracellularly. The reduction or elimination of activity in the formation of binding complexes between the NF-κB pathway-associated protein and the agent being tested can be measured. Thus, the present invention provides a method for screening or assessing a plurality of compounds for their specific binding affinity with NF-κB pathway-associated polypeptides, or a bindable peptide fragment, involving obtaining or providing or testing a plurality of compounds, combining the NF-κB pathway-associated polypeptides, or a bindable peptide fragments, with each of the plurality of compounds for a time sufficient to allow binding under suitable conditions and detecting binding of the NF-κB pathway-associated polypeptides or peptides to each of the plurality of test compounds, thereby identifying the compounds that specifically bind to the NF-κB pathway-associated polypeptides or peptides.

Methods of identifying compounds that modulate the activity of the NF-κB pathway-associated polypeptides and/or peptides comprise combining a potential or candidate compound or drug modulator with an NF-κB pathway-associated polypeptide or peptide, for example, the amino acid sequence encoded by the polynucleotide sequences set forth in Tables 1-6, or peptides encoding sequence thereof, and measuring an effect of the candidate compound or drug modulator on the biological activity of the NF-κB pathway-associated polypeptides or peptides. Such measurable effects include, for example, physical binding interaction; effects on native and cloned polypeptide-expressing cell lines; and effects on components of the NF-κB pathway which are regulated or modulated by the NF-κB pathway-associated polypeptides either directly or indirectly via polypeptide modulators as described herein.

Another method of identifying compounds that modulate the biological activity of the NF-κB pathway-associated proteins comprises combining a potential or candidate compound or drug modulator, e.g., of an NF-κB pathway component with a host cell that expresses the NF-κB pathway-associated polypeptide and measuring an effect of the candidate compound or drug modulator on the biological activity of the polypeptide. The host cell can also be capable of being induced to express the NF-κB pathway-associated polypeptide, e.g., via inducible expression. Physiological effects of a given candidate modulator on the polypeptide can also be measured. Thus, cellular assays for particular NF-κB pathway modulators can be either direct measurement or quantification of the physical biological activity of the NF-κB pathway-associated polypeptide, or they can involve measurement or quantification of a physiological effect. Such methods preferably employ the NF-κB pathway-associated polypeptides as described herein, or an overexpressed recombinant polypeptide in suitable host cells containing an expression vector as described herein, wherein the NF-κB pathway-associated polypeptide is expressed, overexpressed, or undergoes up-regulated expression.

Another aspect of the present invention embraces a method of screening for a compound that is capable of modulating the biological activity of the NF-κB pathway-associated polypeptide, comprising providing a host cell containing an expression vector harboring a nucleic acid sequence encoding a NF-κB pathway-associated polypeptide, or a functional peptide or portion of a NF-κB pathway-associated amino acid sequence as set forth in Tables 1-6; determining the biological activity of the expressed NF-κB pathway-associated polypeptides in the absence of a modulator compound; contacting the cell with the modulator compound; and determining the biological activity of the expressed NF-κB pathway-associated polypeptide in the presence of the modulator compound. In such a method, a difference between the activity of the NF-κB pathway-associated polypeptide in the presence of the modulator compound and in the absence of the modulator compound indicates a modulating effect of the compound.

Essentially any chemical compound can be employed as a potential modulator or ligand in the assays for determining or identifying NF-κB pathway-associated polypeptide modulators or effector molecules. Compounds tested as candidate modulators can be any small chemical compound, or biological entity (e.g., protein, sugar, nucleic acid, lipid). Test compounds are typically small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are routinely run in parallel, for example, in microtiter formats on microtiter plates in robotic assays, e.g., high throughput assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are especially envisioned for the detection of modulators or effectors of the NF-κB pathway-associated polypeptides particularly for preventing, treating or ameliorating NF-κB pathway-related disorders as discussed herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

As is appreciated by the skilled practitioner, a combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT publication no. WO 91/019735), encoded peptides (PCT publication no. WO 93/20242), random bio-oligomers (PCT publication no. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

Solid phase-based in vitro assays in a high throughput format are encompassed in which the cell or tissue expressing an NF-κB pathway-associated polypeptide or peptide is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

Also encompassed are screening and small molecule (e.g., drug) detection assays which involve the detection or identification of small molecules that can bind to the NF-κB pathway-associated polypeptides or peptides. Particularly preferred are assays suitable for high throughput screening methodologies. In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, polypeptides such as NF-κB pathway-associated proteins, based on affmity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify NF-κB pathway-associated polypeptides or peptides for use in measuring or quantifying a biological binding or ligand binding activity, the source may be a whole cell lysate that can be prepared by successive freeze-thaw cycles (e.g., one to three) in the presence of standard protease inhibitors. The NF-κB pathway-associated polypeptides can be partially or completely purified by standard protein purification methods, e.g., affinity chromatography using specific antibody as described, or by ligands specific for an epitope tag engineered into a recombinant polypeptide molecule. Binding activity can then be measured as described.

Compounds that are identified according to the methods provided herein, and that modulate or regulate the biological activity or physiology of the NF-κB pathway-associated polypeptide are embraced as a preferred embodiment of this invention. It is contemplated that such modulatory compounds can be employed in treatment, prevention and therapeutic methods for treating or preventing NF-κB pathway-related disorders or conditions which are mediated by, associated with, regulated or modulated by NF-κB pathway-associated proteins, by administering to an individual in need of such treatment a therapeutically effective amount of the compound identified by the methods described herein. In addition, the present invention provides methods for treating an individual in need of such treatment for NF-κB pathway-related disease, disorder, or condition that is mediated by NF-κB pathway-associated polypeptides, comprising administering to the individual a therapeutically effective amount of the polypeptide-modulating compound identified by a method provided herein.

### Antibodies

The present invention also includes antibodies directed to the NF-κB pathway-associated polypeptides and peptides, as well as methods for the production of such antibodies, including antibodies that specifically recognize one or more epitopes or epitopes of conserved variants, or peptide fragments of NF-κB pathway-associated proteins. Antibodies can be generated against the NF-κB pathway-associated polypeptides comprising, or alternatively, consisting of, an epitope of the polypeptides having the amino acid sequences encoded by the polynucleotides identified in Tables 1-6. Antibodies refer to intact molecules as well as fragments thereof, such as Fab, F(ab′)₂, Fv, which are capable of binding to an epitopic or antigenic determinant. An antigenic determinant refers to that portion of a molecule that makes contact with a particular antibody (i.e., an epitope). The term “epitope” as used herein, refers to portions of a polypeptide having antigenic or immunogenic activity in an animal, preferably a mammal, and most preferably a human. An “immunogenic epitope” as used herein, refers to a portion of a protein that elicits an antibody response in an animal, as determined by any method known in the art, for example, by the methods for generating antibodies described herein. (See, for example, Geysen et al., 1983, Proc. Natl. Acad. Sci. USA, 81:3998-4002). The term “antigenic epitope” as used herein refers to a portion of a protein to which an antibody can immunospecifically bind to its antigen as determined by any method well known in the art, for example, by the immunoassays described herein. Immunospecific binding excludes non-specific binding, but does not necessarily exclude cross-reactivity with other antigens. Antigenic epitopes need not necessarily be immunogenic. Either the full-length protein or an antigenic peptide fragment can be used. Antibodies are preferably prepared from these regions or from discrete fragments in regions of the NF-κB pathway-associated nucleic acid and protein sequences comprising an epitope.

Anti-NF-κB pathway-associated protein antibodies can also be prepared from any region of the NF-κB pathway-associated polypeptide or peptides thereof as described herein. Antibodies can be developed against the entire receptor or portions of the receptor, for example, the intracellular carboxy terminal domain, the amino terminal extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example. Also, when inactivation of the protein is desired, a preferred fragment generates the production of an antibody that diminishes or completely prevents ligand binding.

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 45 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof, as well as any combination of two, three, four, five or more of these antigenic epitopes. Antigenic epitopes are useful, for example, to raise antibodies, including monoclonal antibodies, which specifically bind the epitope. In addition, antigenic epitopes can be used as the target molecules in immunoassays. (See, for instance, Wilson et al., 1984, Cell, 37:767-778; and Sutcliffe et al., 1983, Science, 219:660-666). Such fragments as described herein are not to be construed, however, as encompassing any fragments that may be disclosed prior to the invention.

When the NF-κB pathway-associated polypeptideor a peptide portion thereof is used to immunize a host animal, numerous regions of the polypeptide may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody. Specific binding or specifically binding refer to the interaction between a protein or peptide, i.e., the NF-κB pathway-associated protein or an NF-κB pathway-associated peptide, and a binding molecule, such as an agonist, an antagonist, or an antibody. The interaction is dependent upon the presence of a particular structure (i.e., an antigenic determinant or epitope) of the protein that is recognized by the binding molecule.

Similarly, immunogenic epitopes can be used, for example, to induce antibodies according to methods well known in the art. (See, for instance, Sutcliffe et al., supra; Wilson et al., supra ; Chow et al., 1985, Proc. Natl. Acad Sci. USA, 82:910-914; and Bittle et al., 1985, J. Gen. Virol., 66:2347-2354). Preferred immunogenic epitopes include the immunogenic epitopes disclosed herein, as well as any combination of two, three, four, five or more of these immunogenic epitopes.

The NF-κB pathway-associated polypeptide comprising one or more immunogenic epitopes that elicit an antibody response can be introduced together with a carrier protein, such as albumin, to an animal system (such as rabbit or mouse). Alternatively, if the polypeptide is of sufficient length (e.g., at least about 25 amino acids), the polypeptide can be presented without a carrier. However, immunogenic epitopes comprising as few as 5 to 10 amino acids have been shown to be sufficient to raise antibodies capable of binding to, at the very least, linear epitopes in a denatured polypeptide (e.g., in Western blotting).

An epitope-bearing NF-κB pathway-associated polypeptide or peptide can be used to induce antibodies according to methods well known in the art including, but not limited to, in vivo immunization, in vitro immunization, and phage display methods. See, e.g., Sutcliffe et al., supra; Wilson et al., supra; and Bittle et al., supra). If in vivo immunization is used, animals can be immunized with free peptide; however, the anti-peptide antibody titer may be boosted by coupling the peptide to a macromolecular carrier, such as keyhole limpet hemacyanin (KLH), or tetanus toxoid (TT). For instance, peptides containing cysteine residues can be coupled to a carrier using a linker such as maleimidobenzoyl-N-hydroxysuccinimide ester (MBS), while other peptides may be coupled to carriers using a more general linking agent, such as glutaraldehyde.

Epitope bearing NF-κB pathway-associated polypeptide or peptides can also be synthesized as multiple antigen peptides (MAPs), first described by J. P. Tam et al., 1995, Biomed. Pept., Proteins, Nucleic Acids, 199, 1(3):123-32; and Calvo, et al., 1993, J. Immunol., 150(4):1403-12), which are hereby incorporated by reference in their entirety herein. MAPs contain multiple copies of a specific peptide attached to a non-immunogenic lysine core. MAP peptides usually contain four or eight copies of the peptide, which are often referred to as MAP4 or MAP8 peptides. By way of non-limiting example, MAPs can be synthesized onto a lysine core matrix attached to a polyethylene glycol-polystyrene (PEG-PS) support. The peptide of interest is synthesized onto the lysine residues using 9-fluorenylmethoxycarbonyl (Fmoc) chemistry. For example, Applied Biosystems (Foster City, Calif.) offers commercially available MAP resins, such as, for example, the Fmoc Resin 4 Branch and the Fmoc Resin 8 Branch that can be used to synthesize MAPs. Cleavage of MAPs from the resin is performed with standard trifloroacetic acid (TFA)-based cocktails known in the art. Purification of MAPs, except for desalting, is not generally necessary. MAP peptides can be used in immunizing vaccines that elicit antibodies that recognize both the MAP and the native protein from which the peptide was derived.

Epitope-bearing NF-κB pathway-associated polypeptides and peptides thereof can also be incorporated into a coat protein of a virus, which can then be used as an immunogen or a vaccine with which to immunize animals, including humans, in order stimulate the production of anti-epitope antibodies. For example, the V3 loop of the gp120 glycoprotein of the human immunodeficiency virus type 1 (HIV-1) has been engineered to be expressed on the surface of rhinovirus. Immunization with rhinovirus displaying the V3 loop peptide yielded apparently effective mimics of the HIV-1 immunogens (as measured by their ability to be neutralized by anti-HIV-1 antibodies as well as by their ability to elicit the production of antibodies capable of neutralizing HIV-1 in cell culture). This techniques of using engineered viral particles as immunogens is described in more detail in Smith et al., 1997, Behring Inst Mitt Feb, (98):229-39; Smith et al., 1998, J. Virol., 72:651-659; and Zhang et al., 1999, Biol. Chem., 380:365-74), which are hereby incorporated by reference herein in their entireties.

Epitope bearing NF-κB pathway-associated polypeptides and peptides thereof can be modified, for example, by the addition of amino acids at the amino- and/or carboxy-terminus of the peptide. Such modifications are performed, for example, to alter the conformation of the epitope bearing polypeptides such that the epitope will have a conformation more closely related to the structure of the epitope in the native protein. An example of a modified epitope-bearing polypeptide of the invention is a polypeptide in which one or more cysteine residues have been added to the polypeptide to allow for the formation of a disulfide bond between two cysteines, thus resulting in a stable loop structure of the epitope-bearing polypeptide under non-reducing conditions. Disulfide bonds can form between a cysteine residue added to the polypeptide and a cysteine residue of the naturally-occurring epitope, or between two cysteines which have both been added to the naturally-occurring epitope-bearing polypeptide. In addition, it is possible to modify one or more amino acid residues of the naturally-occurring epitope-bearing polypeptide by substitution with cysteines to promote the formation of disulfide bonded loop structures. Cyclic thioether molecules of synthetic peptides can be routinely generated using techniques known in the art, e.g., as described in PCT publication WO 97/46251, incorporated in its entirety by reference herein. Other modifications of epitope-bearing polypeptides contemplated by this invention include biotinylation.

For the production of antibodies in vivo, host animals, such as rabbits, rats, mice, sheep, or goats, are immunized with either free or carrier-coupled peptides or MAP peptides, for example, by intraperitoneal and/or intradermal injection. Injection material is typically an emulsion containing about 100 μg of peptide or carrier protein and Freund's adjuvant, or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal can be increased by selection of anti-peptide antibodies, e.g., by adsorption of the peptide onto a solid support and elution of the selected antibodies according to methods well known in the art.

As one having skill in the art will appreciate, and as discussed above, the NFκB pathway-associated polypeptides and peptides as described herein, which comprise an immunogenic or antigenic epitope, can be fused to other polypeptide sequences. For example, the polypeptides of the present invention can be fused with the constant domain of immunoglobulins (IgA, IgE, IgG, IgD, or IgM), or portions thereof, e.g., CH1, CH2, CH3, or any combination thereof, and portions thereof, or with albumin (including, but not limited to, recombinant human albumin, or fragments or variants thereof (see, e.g., U. S. Pat. No. 5,876,969; EP Patent No. 0 413 622; and U.S. Pat. No. 5,766,883, incorporated by reference in their entirety herein), thereby resulting in chimeric polypeptides. Such fusion proteins may facilitate purification and may increase half-life in vivo. This has been shown for chimeric proteins containing the first two domains of the human CD4-polypeptide and various domains of the constant regions of the heavy or light chains of mammalian immunoglobulins. See, e.g., Traunecker et al., 1988, Nature, 331:84-86).

Enhanced delivery of an antigen across the epithelial barrier to the immune system has been demonstrated for antigens (e.g., insulin) conjugated to an FcRn binding partner, such as IgG or Fc fragments (see, e.g., PCT publications WO 96/22024 and WO 99/04813). IgG fuision proteins that have a disulfide-linked dimeric structure due to the IgG portion disulfide bonds have also been found to be more efficient in binding and neutralizing other molecules than are monomeric polypeptides, or fragments thereof, alone. See, e.g., Fountoulakis et al., 1995, J. Biochem., 270:3958-3964).

Nucleic acids encoding epitopes can also be recombined with a gene of interest as an epitope tag (e.g., a hemagglutinin (“HA”) tag or Flag tag) to aid in detection and purification of the expressed polypeptide. For example, a system for the ready purification of non-denatured fusion proteins expressed in human cell lines has been described by Janknecht et al., (1991, Proc. Natl. Acad. Sci. USA, 88:8972-897). In this system, the gene of interest is subcloned into a vaccinia recombination plasmid such that the open reading frame of the gene is translationally fused to an amino-terminal tag having six histidine residues. The tag serves as a matrix binding domain for the fusion protein. Extracts from cells infected with the recombinant vaccinia virus are loaded onto an Ni²⁺ nitriloacetic acid-agarose column and histidine-tagged proteins are selectively eluted with imidazole-containing buffers.

Additional fusion proteins of the invention can be generated by employing the techniques of gene-shuffling, motif-shuffling, exon-shuffling, and/or codon-shuffling (collectively referred to as “DNA shuffling”). DNA shuffling can be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol., 16(2):76-82; Hansson, et al., 1999, J. Mol. Biol., 287:265-76; and Lorenzo and Blasco, 1998, Biotechniques, 24(2):308-313, the contents of each of which are hereby incorporated by reference in its entirety).

In one aspect, the alteration of a polynucleotide encoding the NF-κB pathway-associated polypeptides or fragments thereof can be achieved by DNA shuffling. DNA shuffling involves the assembly of two or more DNA segments by homologous or site-specific recombination to generate variation in the polynucleotide sequence. Alternatively, the NF-κB pathway-associated polynucleotides, or their encoded polypeptides or peptides, can be altered by being subjected to random mutagenesis by error-prone PCR, random nucleotide insertion, or other methods, prior to recombination. In addition, one or more components, motifs, sections, parts, domains, fragments, etc., of polynucleotides encoding the NF-κB pathway-associated polypeptides can be recombined with one or more components, motifs, sections, parts, domains, fragments, etc. of one or more heterologous molecules.

A bispecific or bifunctional antibody is an artificial hybrid antibody having two different heavy/light chain pairs and two different binding sites. Bispecific antibodies can be produced by a variety of methods, including fusion of hybridomas or linking of Fab′ fragments. (See, e.g., Songsivilai & Lachmann, 1990, Clin. Exp. Immunol., 79:315-321; Kostelny et al., 1992, J. Immunol., 148:1547 1553). In addition, bispecific antibodies can be formed as “diabodies” (See, Holliger et al., 1993, Proc. Natl. Acad. Sci. USA, 90:6444-6448), or “Janusins” (See, Traunecker et al., 1991, EMBO J., 10:3655-3659 and Traunecker et al., 1992, Int. J. Cancer Suppl. 7:51-52 -127).

Antibodies of the invention include the various types mentioned herein above, as well as anti-idiotypic (anti-Id) antibodies (including, e.g., anti-Id antibodies to antibodies of the invention), intracellularly made antibodies (i.e., intrabodies), and epitope-binding fragments of any of the above. The immunoglobulin molecules of the invention can be of any type (e.g., IgG, IgE, IgM, IgD, IgA and IgY), class or subclass (e.g., IgG1, IgG2, IgG3, IgG4, IgA1 and IgA2) of immunoglobulin molecule. A preferred immunoglobulin is of the IgG1 isotype. Other preferred antibody isotypes include the IgG2 and the IgG4 isotypes.

As is appreciated by the skilled practitioner, immunoglobulins can have both a heavy and a light chain. An array of IgG, IgE, IgM, IgD, IgA, and IgY heavy chains can be paired with a light chain of the kappa or lambda types. Most preferably, antibodies of the present invention are human antigen-binding antibodies and antibody fragments and include, but are not limited to, Fab, Fab′ F(ab′) 2, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of the following: hinge region, and CH1, CH2, and CH3 domains. Also included in connection with the invention are antigen-binding fragments also comprising any combination of variable region(s) with a hinge region, and CH1, CH2, and CH3 domains. The antibodies of the invention can be from any animal origin including birds and mammals. Preferably, the antibodies are of human, murine (e.g., mouse and rat), donkey, sheep, rabbit, goat, guinea pig, camel, horse, or chicken origin. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described herein and, for example, in U.S. Pat. No. 5,939,598.

The antibodies of the present invention can be monospecific, bispecific, trispecific, or of greater multispecificity. Multispecific antibodies can be specific for different epitopes of the NF-κB pathway-associated polypeptides, or can be specific for both an NF-κB pathway-associated polypeptide and a heterologous epitope, such as a heterologous polypeptide or solid support material. (See, e.g., PCT publications WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553).

Antibodies of the present invention can be described or specified in terms of the epitope(s) or portion(s) of the NF-κB pathway-associated polypeptides that are recognized or specifically bound. The epitope(s) or polypeptide portion(s) can be specified, e.g., by N-terminal and C-terminal positions, by size in contiguous amino acid residues, or as presented in the sequences defined herein. Further included in accordance with the present invention are antibodies which bind to polypeptides encoded by polynucleotides which hybridize to the NF-κB pathway-associated polynucleotides shown in Tables 1-6 under stringent, or moderately stringent, hybridization conditions as described herein.

The antibodies of the invention (including molecules comprising, or alternatively consisting of, antibody fragments or variants thereof) can bind immunospecifically and/or preferentially to a NF-κB pathway-associated polypeptide, an NF-κB pathway-associated polypeptide fragment, or a variant NF-κB pathway-associated protein. By way of non-limiting example, an antibody can be considered to bind to a first antigen preferentially if it binds to the first antigen with a dissociation constant (Kd) that is less than the antibody's Kd for the second antigen. In another non-limiting embodiment, an antibody can be considered to bind to a first antigen preferentially if it binds to the first antigen with an affinity that is at least one order of magnitude less than the antibody's Ka for the second antigen. In another non-limiting example, an antibody can be considered to bind to a first antigen preferentially if it binds to the first antigen with an affinity that is at least two orders of magnitude less than the antibody's Kd for the second antigen.

In another nonlimiting example, an antibody can be considered to bind to a first antigen preferentially if it binds to the first antigen with an off rate (koff) that is less than the antibody's koff for the second antigen. In a further nonlimiting example, an antibody can be considered to bind to a first antigen preferentially if it binds to the first antigen with an affinity that is at least one order of magnitude less than the antibody's koff for the second antigen. In yet a further nonlimiting example, an antibody can be considered to bind to a first antigen preferentially if it binds to the first antigen with an affinity that is at least two orders of magnitude less than the antibody's koff for the second antigen.

Antibodies against the NF-κB pathway-associated polypeptides of this invention can also be described or specified in terms of their binding affinity to the NF-κB pathway-associated polypeptides or peptides thereof. Preferred binding affinities include those with a dissociation constant or Kd of less than 5×10⁻² M, 1×10⁻² M, 5×10⁻³ M, 1×10⁻³ M, 5×10⁻⁴ M, or 1×10⁻⁴ M. More preferred binding affinities include those with a dissociation constant or Kd less than 5×10⁻⁵ M, 1×10⁻⁵M, 5×10⁻⁶ M, 1×10⁻⁶ M, 5×10⁻⁷ M, 1×10⁻⁷ M, 5×10⁻⁸ M, or 1×10⁻⁸ M, Even more preferred antibody binding affinities include those with a dissociation constant or Kd of less than 5×10⁻⁹ M, 1×10⁻⁹ M, 5×10⁻¹⁰ M, 1×10⁻¹⁰ M, 5×10⁻¹¹ M, 1×10^{-11 M,} 5×10⁻¹² M, 1×10⁻¹² M, 5×10⁻¹³ M, 1×10⁻¹³ M, 5×10⁻¹⁴ M, 1×10¹⁴ M, 5×10⁻¹⁵ M, or 1×10⁻¹⁵ M.

More specifically, antibodies of the invention bind to the NF-κB pathway-associated polypeptides, fragments, or variants thereof, with an off rate (koff) of less than or equal to about 5×10⁻² sec⁻¹, 1×10⁻² sec⁻¹,5×10⁻³ sec⁻¹, or 1×10⁻³ sec⁻¹. More preferably, antibodies of the invention bind to the NF-κB pathway-associated polypeptides, fragments, or variants thereof, with an off rate (koff) of less than or equal to about 5×10⁻⁴ sec⁻¹, 1×10⁻⁴ sec⁻¹, 5×10⁻⁵ sec⁻¹, 1×10⁻⁵ sec⁻¹, 5×10⁻⁶ sec⁻¹, 1×10⁻⁶ sec⁻¹, 5×10⁻⁷ sec⁻¹, or 1×10⁻⁷ sec⁻¹. In other aspects, antibodies invention bind to the NF-κB pathway-associated polypeptides, fragments, or variants thereof with an on rate (kon) of greater than or equal to 1×10³ M⁻¹ sec⁻¹, 5×10³ M⁻¹ sec⁻¹, 1×10⁴ M⁻¹ sec⁻¹, or 5×10⁴ M⁻¹ sec⁻¹. More preferably, antibodies of the invention bind to the NF-κB pathway-associated polypeptides, or fragments, or variants thereof with an on rate greater than or equal to 1×10⁵ M⁻¹ sec⁻¹, 5×10⁵ M⁻¹ sec⁻¹, 1×10⁶ M⁻¹ sec⁻, 5×10⁻⁶ M⁻¹ sec⁻¹, or 1×10⁻⁷ M⁻¹ sec⁻¹.

The present invention also provides antibodies that competitively inhibit the binding of an antibody to an NF-κB pathway-associated polypeptide epitope as determined by any method known in the art for determining competitive binding, for example, the immunoassays as described herein. In preferred embodiments, the antibody competitively inhibits binding to an epitope by at least 95%, at least 90%, at least 85%, at least 80%, at least 75%, at least 70%, at least 60%, or at least 50%.

As mentioned above, antibodies of the present invention can act as agonists or antagonists of the NF-κB pathway-associated polypeptides. For example, the invention includes antibodies that can disrupt receptor/ligand interactions, or disrupt interactions of cellular molecules affected by NF-κB pathway-associated polypeptides following cell stimulation, either partially or fully. The invention includes both receptor-specific antibodies and ligand-specific antibodies. The invention also includes receptor-specific antibodies that do not prevent ligand binding, but do prevent receptor activation. Receptor activation (i.e., signaling) can be determined by techniques described herein or as otherwise known in the art. For example, receptor activation can be determined by detecting the phosphorylation (e.g., on tyrosine or serine/threonine) of the receptor or its substrate by immunoprecipitation followed by Western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in the absence of the antibody.

In an embodiment of the present invention, antibodies that immunospecifically bind to the NF-κB pathway-associated polypeptides, or to a fragment or variant thereof, comprise a polypeptide having the amino acid sequence of any one of the Ig heavy chains expressed by an NF-κB pathway-associated polypeptide antibody-expressing cell line of the invention, and/or any one of the Ig light chains expressed by an NF-κB pathway-associated polypeptide antibody-expressing cell line of the invention. In another embodiment of the present invention, antibodies that immunospecifically bind to a NF-κB pathway-associated polypeptide, or to a fragment or variant thereof, comprise a polypeptide having the amino acid sequence of any one of the V_{H} domains of a heavy chain expressed by an anti-NF-κB pathway-associated protein antibody-expressing cell line, and/or any one of the V_{L} domains of a light chain expressed by an anti-NF-κB pathway-associated protein antibody-expressing cell line. In preferred embodiments, antibodies of the present invention comprise the amino acid sequence of a V_{H} domain and V_{L} domain expressed by a single anti-NF-κB pathway-associated protein antibody-expressing cell line. In alternative embodiments, antibodies of the present invention comprise the amino acid sequence of a V_{H} domain and a V_{L} domain expressed by two different anti-NF-κB pathway-associated protein antibody-expressing cell lines. Molecules comprising, or alternatively consisting of, antibody fragments or variants of the V_{H} and/or V_{L} domains expressed by an anti-NF-κB pathway-associated protein antibody-expressing cell line that immunospecifically bind to the NF-κB pathway-associated protein are also encompassed by the invention, as are nucleic acid molecules encoding these V_{H} and V_{L} domains, molecules, fragments and/or variants.

The present invention also provides antibodies that immunospecificially bind to the NF-κB pathway-associated polypeptides, or fragment or variant of the polypeptides, wherein the antibodies comprise, or alternatively consist of, a polypeptide having an amino acid sequence of any one, two, three, or more of the V_{H}CDRs contained in an Ig heavy chain expressed by one or more anti-NF-κB pathway-associated polypeptide antibody expressing cell lines. In particular, the invention provides antibodies that immunospecifically bind to the NF-κB pathway-associated polypeptides, comprising, or alternatively consisting of, a polypeptide having the amino acid sequence of a V_{H} CDR1 contained in an Ig heavy chain expressed by one or more anti-NF-κB pathway-associated polypeptides antibody expressing cell lines. In another embodiment, antibodies that immunospecifically bind to the NF-κB pathway-associated polypeptides, comprise, or alternatively consist of, a polypeptide having the amino acid sequence of a V_{H} CDR2 contained in a heavy chain expressed by one or more anti-NF-κB pathway-associated polypeptide antibody expressing cell lines. In a preferred embodiment, antibodies that immunospecifically bind to the NF-κB pathway-associated proteins, comprise, or alternatively consist of, a polypeptide having the amino acid sequence of a V_{H} CDR3 contained in an Ig heavy chain expressed by one or more anti-NF-κB pathway-associated polypeptide antibody expressing cell lines of the invention. Molecules comprising, or alternatively consisting of, these antibodies, or antibody fragments or variants thereof, that immunospecifically bind to the NF-κB pathway-associated polypeptides or to a protein fragment or variant thereof are also encompassed by the invention, as are nucleic acid molecules encoding these anti-NF-κB pathway-associated polypeptide antibodies, molecules, fragments and/or variants.

The present invention also provides antibodies that immunospecificially bind to the NF-κB pathway-associated polypeptides, or a fragment or variant of the proteins, wherein the antibodies comprise, or alternatively consist of, a polypeptide having an amino acid sequence of any one, two, three, or more of the V_{L} CDRs contained in an Ig heavy chain expressed by one or more anti-NF-κB pathway-associated polypeptide antibody expressing cell lines of the invention. In particular, the invention provides antibodies that immunospecifically bind to the polypeptides, comprising, or alternatively consisting of, a polypeptide having the amino acid sequence of a V_{L} CDR1 contained in an Ig heavy chain expressed by one or more anti-NF-κB pathway-associated polypeptide antibody-expressing cell lines of the invention. In another embodiment, antibodies that immunospecifically bind to the NF-κB pathway-associated polypeptides, comprise, or alternatively consist of, a polypeptide having the amino acid sequence of a V_{L} CDR2 contained in an Ig heavy chain expressed by one or more anti-NF-κB pathway-associated polypeptide antibody-expressing cell lines of the invention. In a preferred embodiment, antibodies that immunospecifically bind to the NF-κB pathway-associated polypeptide, comprise, or alternatively consist of, a polypeptide having the amino acid sequence of a V_{L} CDR3 contained in an Ig heavy chain expressed by one or more anti-NF-κB pathway-associated polypeptide antibody-expressing cell lines of the invention. Molecules comprising, or alternatively consisting of, these antibodies, or antibody fragments or variants thereof, that immunospecifically bind to the NF-κB pathway-associated polypeptides or to a protein fragment or variant thereof are also encompassed by the invention, as are nucleic acid molecules encoding these anti-NF-κB pathway-associated polypeptide antibodies, molecules, fragments and/or variants.

The present invention also provides antibodies (including molecules comprising, or alternatively consisting of, antibody fragments or variants) that immunospecifically bind to the NF-κB pathway-associated polypeptides or to an polypeptide fragment or variant, wherein the antibodies comprise, or alternatively consist of, one, two, three, or more V_{H} CDRs, and one, two, three or more V_{L} CDRs, as contained in an Ig heavy chain or light chain expressed by one or more anti-NF-κB pathway-associated polypeptide antibody-expressing cell lines of the invention. In particular, the invention provides antibodies that immunospecifically bind to the NF-κB pathway-associated polypeptides or to a polypeptide fragment or variant, wherein the antibodies comprise, or alternatively consist of, a V_{H} CDR1 and a V_{L} CDR1, a V_{H} CDR1 and a V_{L} CDR2, a V_{H} CDR1 and a V_{L} CDR3, a V_{H} CDR2 and a V_{L} CDR1, VH CDR2 and V_{L} CDR2, a V_{H} CDR2 and a V_{L} CDR3, a V_{H} CDR3 and a V_{H} CDR1, a V_{H} CDR3 and a V_{L} CDR2, a V_{H} CDR3 and a V_{L} CDR3, or any combination thereof, of the V_{H} CDRs and V_{L} CDRs contained in an Ig heavy chain or Ig light chain expressed by one or more anti-NF-κB pathway-associated polypeptide antibody-expressing cell lines of the invention. In a preferred embodiment, one or more of these combinations are from a single anti-NF-κB pathway-associated polypeptide antibody-expressing cell line. Molecules comprising, or alternatively consisting of, fragments or variants of these antibodies that immunospecifically bind to the NF-κB pathway-associated polypeptides are also encompassed by the invention, as are nucleic acid molecules encoding these anti-NF-κB pathway-associated polypeptide antibodies, molecules, fragments or variants.

Also provided are nucleic acid molecules, generally isolated, encoding an antibody of the invention (including molecules comprising, or alternatively consisting of, antibody fragments or variants thereof). In a specific aspect, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of an immunoglobulin heavy chain expressed by an anti-NF-κB pathway-associated polypeptides antibody-expressing cell line of the invention and a V_{L} domain having an amino acid sequence of an immunoglobulin light chain expressed by an anti-NF-κB pathway-associated polypeptide antibody-expressing cell line of the invention. In another aspect, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of an immunoglobulin heavy chain expressed by an anti-NF-κB pathway-associated polypeptide antibody-expressing cell line of the invention, or a V_{L} domain having an amino acid sequence of a light chain expressed by an anti-polypeptide antibody-expressing cell line of the invention. The present invention also provides antibodies that comprise, or alternatively consist of, variants (including derivatives) of the antibody molecules (e.g., the V_{H} domains and/or V_{L} domains) described herein, which antibodies immunospecifically bind to the NF-κB pathway-associated polypeptides or to a fragment or a variant thereof.

Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably the molecules are immunoglobulin molecules. Also, preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions, relative to the reference V_{H} domain, V_{H} CDR1, V_{H} CDR2, V_{H} CDR3, V_{L} domain, V_{L} CDR1, V_{L} CDR2, or V_{L} CDR3 domain.

A “conservative amino acid substitution” is one in which the amino acid residue is replaced with an amino acid residue having a side chain with a similar charge. Families of amino acid residues-having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Alternatively, mutations can be introduced randomly along all or part of the coding sequence, such as by saturation mutagenesis. The resultant mutants can be screened for biological activity to identify mutants that retain activity. For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or to improve hybridoma antibody production. Alternatively, non-neutral missense mutations can alter an antibody's ability to bind antigen. The location of most silent and neutral missense mutations is likely to be in the framework regions, while the location of most non-neutral missense mutations is likely to be in the CDRs, although this is not an absolute requirement. One of skill in the art is able to design and test mutant molecules with desired properties, such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein can be determined using techniques described herein or by routinely modifying techniques known and practiced in the art.

In a specific aspect, an antibody of the invention (including a molecule comprising, or alternatively consisting of, an antibody fragment or variant thereof), that immunospecifically binds to the NF-κB pathway-associated polypeptides or to fragments or variants thereof, comprises, or alternatively consists of, an amino acid sequence encoded by a nucleotide sequence that hybridizes to a nucleotide sequence that is complementary to that encoding one of the V_{H} or V_{L} domains expressed by one or more anti-NF-κB pathway-associated protein antibody-expressing cell lines of the invention, preferably under stringent conditions, e.g., hybridization to filter-bound DNA in 6× sodium chloride/sodium citrate (SSC) at about 45° C. followed by one or more washes in 0.2×SSC/0.1% SDS at about 50-65° C., preferably under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structures and many of the same biological activities. Thus, in one aspect, an antibody (including a molecule comprising, or alternatively consisting of, an antibody fragment or variant thereof), that immunospecifically binds to the NF-κB pathway-associated polypeptides, or to peptide fragments or variants, comprises, or alternatively consists of, a V_{H} domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a V_{H} domain of a heavy chain expressed by an anti-NF-κB pathway-associated polypeptide antibody-expressing cell line of the invention.

In another aspect, an antibody (including a molecule comprising, or alternatively consisting of, an antibody fragment or variant thereof), that immunospecifically binds to the NF-κB pathway-associated polypeptide or to fragments or variants, comprises, or alternatively consists of, a V_{L} domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a V_{L} domain of a light chain expressed by an anti-NF-κB pathway-associated polypeptide antibody-expressing cell line of the invention.

In another preferred aspect, an antibody that enhances the activity of the NF-κB pathway-associated polypeptides, or a fragment or variant thereof, comprises, or alternatively consists of, a polypeptide having the amino acid sequence of a V_{L} CDR3 of an antibody of the invention, or a fragment or variant thereof. Nucleic acid molecules encoding these antibodies are also encompassed by the invention.

In addition, as nonlimiting examples, anti-NF-κB pathway-associated polypeptide antibodies as described herein can be used to purify, detect, and target the polypeptides, including both in vitro and in vivo diagnostic, detection, screening, and/or therapeutic methods. For example, the antibodies can be used in immunoassays for qualitatively and quantitatively measuring levels of the NF-κB pathway-associated polypeptides in biological samples. (See, e.g., Harlow et al., Antibodies: A Laboratory Manual, Cold Spring Harbor Laboratory Press, 2nd Ed. 1988, which is incorporated by reference herein in its entirety). By way of another nonlimiting example, anti-NF-κB pathway-associated polypeptide antibodies can be administered to individuals as a form of passive immunization. Alternatively, antibodies of the present invention can be used for epitope mapping to identify the epitope(s) that are bound by one or more antibodies. Epitopes identified in this way can, in turn, for example, be used as vaccine candidates, i.e., to immunize an individual to elicit antibodies against the naturally-occurring forms of the NF-κB pathway-associated polypeptides.

As discussed in more detail below, anti-NF-κB pathway-associated polypeptide antibodies can be used either alone or in combination with other compositions. The antibodies can further be recombinantly fused to a heterologous polypeptide at the N- or C-terminus, or chemically conjugated (including covalent and non-covalent conjugations) to polypeptides or other compositions. For example, antibodies of the present invention can be recombinantly fused or conjugated to molecules that are useful as labels in detection assays and to effector molecules such as heterologous polypeptides, drugs, radionuclides, or toxins. See, e.g., PCT publications WO 92/08495; WO 91/14438; WO 89/12624; U.S. Pat. No. 5,314,995 and EP 396, 387.

The antibodies of the invention further include derivatives that are modified, i.e., by the covalent attachment of any type of molecule to the antibody. For example, without limitation, anti-NF-κB pathway-associated polypeptide antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. In addition, the derivative can contain one or more non-classical amino acids.

Antibodies against the NF-κB pathway-associated polypeptides of the present invention can be generated by any suitable method known in the art. Polyclonal antibodies directed against an antigen or immunogen of interest can be produced by various procedures well known in the art. For example, the NF-κB pathway-associated polypeptides or peptide can be administered to various host animals as elucidated above to induce the production of sera containing polyclonal antibodies specific for the antigen. Various adjuvants may be used to increase the immunological response, depending on the host species; adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art, including the use of hybridoma, recombinant and phage display technologies, or a combination thereof. For example, monoclonal antibodies can be produced using hybridoma techniques as known and practiced in the art and as taught, for example, in Harlow et al., Antibodies: A Laboratory Manual, (Cold Spring Harbor Laboratory Press, 2nd Ed. 1988; Hammerling, et al., In: Monoclonal Antibodies and T-Cell Hybridomas, Elsevier, N.Y., pages 563-681, 1981, the contents of which are incorporated herein by reference in their entireties. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a nonlimiting example, mice can be immunized with a NF-κB pathway-associated polypeptide or a peptide thereof, or with a cell expressing the polypeptide or peptide. Once an immune response is detected, e.g., antibodies specific for the antigen are detected in the sera of immunized mice, the spleen is harvested and splenocytes are isolated. The splenocytes are then fused by well known techniques to any suitable myeloma cells, for example cells from cell line SP2/0 or P3X63-AG8.653 available from the ATCC. Hybridomas are selected and cloned by limiting dilution techniques. The hybridoma clones are then assayed by methods known in the art to determine and select those cells that secrete antibodies capable of binding to the NF-κB pathway-associated polypeptide, or to a portion of the polypeptide. Ascites fluid, which generally contains high levels of antibodies, can be generated by immunizing mice with positive hybridoma clones.

Another well known method for producing both polyclonal and monoclonal human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds., 1994, John Wiley & Sons, New York, which is hereby incorporated by reference herein in its entirety. The source of B cells for transformation is commonly human peripheral blood, but B cells for transformation can also be obtained from other sources including, but not limited to, lymph node, tonsil, spleen, tumor tissue, and infected tissues. Tissues are generally prepared as single cell suspensions prior to EBV transformation. In addition, T cells that may be present in the B cell samples can be either physically removed or inactivated (e.g., by treatment with cyclosporin A). The removal of T cells is often advantageous, because T cells from individuals who are seropositive for anti-EBV antibodies can suppress B cell immortalization by EBV. In general, a sample containing human B cells is innoculated with EBV and cultured for 3-4 weeks. A typical source of EBV is the culture supernatant of the B95-8 cell line (ATCC; VR-1492). Physical signs of EBV transformation can generally be seen toward the end of the 3-4 week culture period.

By phase-contrast microscopy, transformed cells appear large, clear and “hairy”; they tend to aggregate in tight clusters of cells. Initially, EBV lines are generally polyclonal. However, over prolonged periods of cell culture, EBV lines can become monoclonal as a result of the selective outgrowth of particular B cell clones. Alternatively, polyclonal EBV transformed lines can be subcloned (e.g., by limiting dilution) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human x mouse ; e.g., SPAM-8, SBC-H20, and CB-F7), and human cell lines (e.g., GM 1500, SKO-007, RPMI 8226, and KR-4). Thus, the present invention also includes a method of generating polyclonal or monoclonal human antibodies against polypeptides of the invention or fragments thereof, comprising EBV-transformation of human B cells.

Antibody fragments that recognize specific epitopes can be generated by known techniques. For example, Fab and F(ab′)2 fragments of the invention can be produced by proteolytic cleavage of immunoglobulin molecules, using enzymes such as papain (to produce Fab fragments) or pepsin (to produce F (ab′) 2 fragments). F(ab′)2 fragments contain the variable region, the light chain constant region and the CH1 domain of the heavy chain.

Antibodies encompassed by the present invention can also be generated using various phage display methods known in the art. In phage display methods, functional antibody domains are displayed on the surface of phage particles that carry the polynucleotide sequences encoding them. In a particular embodiment, such phage can be utilized to display antigen-binding domains expressed from a repertoire or combinatorial antibody library (e.g., human or murine). Phage expressing an antigen binding domain that binds to the antigen of interest, i.e., the NF-κB pathway-associated polypeptide or fragment thereof, can be selected or identified with antigen, e.g., using labeled antigen or antigen bound or captured onto a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., 1995, J. Immunol. Methods, 182:41-50; Ames et al., 1995, J. Immunol. Methods, 184:177-186; Kettleborough et al., 1994, Eur. J. Immunol., 24:952-958; Persic et al., 1997, Gene, 187:9-18; Burton et al., 1994, Advances in Immunology, 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below.

Examples of techniques that can be used to produce single-chain Fvs and antibodies include those described in U.S. Pat. Nos. 4,946,778 and 5,258,498; Huston et al., 1991, Methods in Enzymology, 203:46-88; Shu et al., 1993, Proc. Natl. Acad. Sci. USA, 90:7995-7999; and Skerra et al., 1988, Science, 240:1038-1040. For some uses, including the in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. (See, e.g., Morrison, 1985, Science, 229:1202; Oi et al., 1986, BioTechniques, 4:214; Gillies et al., 1989, J. Immunol. Methods, 125:191-202; and U.S. Pat. Nos. 5,807,715; 4,816,567; and 4,816,397, which are incorporated herein by reference in their entirety).

Humanized antibodies are antibody molecules from non-human species antibody that bind to the desired antigen and have one or more complementarity determining regions (CDRs) from the nonhuman species and framework regions from a human immnunoglobulin molecule. Often, framework residues in the human framework regions are substituted with the corresponding residues from the CDR donor antibody to alter, and preferably to improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, 1991, Molecular Immunology, 28(4/5):489-498; Studnicka et al., 1994, Protein Engineering, 7(6):805-814; Roguska et al., 1994, Proc. Natl. Acad. Sci. USA, 91:969-973; and chain shuffling (U.S. Pat. No. 5,565,332).

Completely human antibodies can be made by a variety of methods known in the art, including the phage display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. Completely human antibodies are particularly desirable for therapeutic treatment of human patients, so as to avoid or alleviate immune reaction to foreign protein.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly, or by homologous recombination, into mouse embryonic stem cells. Alternatively, the human variable region, constant region, and diversity region may be introduced into mouse embryonic stem cells, in addition to the human heavy and light chain genes. The mouse heavy and light chain immunoglobulin genes can be rendered nonfunctional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a polypeptide of the invention.

Monoclonal antibodies directed against the antigen can be obtained from the immunized transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce useful human IgG, IgA, IgM and IgE antibodies. For an overview of the technology for producing human antibodies, see Lonberg and Huszar, 1995, Intl. Rev. Immunol., 13:65-93. For a detailed discussion of the technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, which are incorporated by reference herein in their entirety. In addition, companies such as Abgenix, Inc. (Fremont, Calif.) and Genpharm (San Jose, Calif.) can be engaged to provide human antibodies directed against a selected antigen using technology similar to the above-described technologies.

In another aspect, completely human antibodies that recognize a selected epitope can be generated using a technique referred to as “guided selection”. In this approach, a selected non-human monoclonal antibody, e.g., a mouse antibody, is used to guide the selection of a completely human antibody recognizing the same epitope. (Jespers et al., 1988, BioTechnology, 12:899-903).

Further, antibodies specific for the NF-κB pathway-associated polypeptide can, in turn, be utilized to generate anti-idiotypic antibodies that “mimic” the polypeptide using techniques well known to those skilled in the art. (See, e.g., Greenspan and Bona, 1989, FASEB J., 7(5):437-444 and Nissinoff, 1991, J. Immunol., 147(8):2429-2438). For example, antibodies which bind to and competitively inhibit polypeptide multimerization and/or binding of the NF-κB pathway-associated polypeptide to a ligand can be used to generate anti-idiotypic antibodies that “mimic” the polypeptide multimerization and/or binding domain and, as a consequence, bind to and neutralize the polypeptide and/or its ligand, e.g., in therapeutic regimens. Such neutralizing anti-idiotypes or Fab fragments of such anti-idiotypes can be used to neutralize polypeptide ligand. For example, such anti-idiotypic antibodies can be used to bind the NF-κB pathway-associated polypeptides and/or to bind their ligands/receptors, and thereby activate or block their biological activity.

In another aspect, intrabodies are embraced. Intrabodies are antibodies, often scFvs, that are expressed from a recombinant nucleic acid molecule and are engineered to be retained intracellularly (e.g., retained in the cytoplasm, endoplasmic reticulum, or periplasm of the host cells). Intrabodies can be used, for example, to ablate the function of a protein to which the intrabody binds. The expression of intrabodies can also be regulated through the use of inducible promoters in the nucleic acid expression vector comprising nucleic acid encoding the intrabody. Intrabodies of the invention can be produced using methods known in the art, such as those disclosed and reviewed in Chen et al., 1994, Hum. Gene Ther., 5:595-601; Marasco, W. A., 1997, Gene Ther., 4:11-15; Rondon and Marasco, 1997, Annu. Rev. Microbiol., 51:257-283; Proba et al., 1998, J. Mol. Biol., 275:245-253; Cohen et al., 1998, Oncogene, 17:2445-2456; Ohage and Steipe, 1999, J. Mol. Biol., 291:1119-1128; Ohage et al., 1999, J. Mol. Biol., 291:1129-1134; Wirtz and Steipe, 1999, Protein Sci., 8:2245-2250; Zhu et al., 1999, J. Immunol. Methods, 231:207-222.

XenoMouse Technology Antibodies in accordance with the invention are preferably prepared by the utilization of a transgenic mouse that has a substantial portion of the human antibody producing genome inserted, but that is rendered deficient in the production of endogenous murine antibodies (e.g., XenoMouse strains available from Abgenix Inc., Fremont, Calif.). Such mice are capable of producing human immunoglobulin molecules and antibodies and are virtually deficient in the production of murine immunoglobulin molecules and antibodies. Technologies utilized for achieving the same are disclosed in the patents, applications, and references disclosed herein.

The ability to clone and reconstruct megabase-sized human loci in YACs and to introduce them into the mouse germline provides a powerful approach to elucidating the functional components of very large or crudely mapped loci, as well as generating useful models of human disease. Furthermore, the utilization of such technology for substitution of mouse loci with their human equivalents can provide unique insights into the expression and regulation of human gene products during development, their communication with other systems, and their involvement in disease induction and progression. An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies, as well as their role in B cell development. Furthermore, such a strategy can provide an ideal source for the production of fully human monoclonal antibodies (Hu MAbs) an important milestone toward fulfilling the promise of antibody therapy in human disease.

Fully human antibodies are expected to minimize the immunogenic and allergic responses intrinsic to mouse or mouse-derivatized monoclonal antibodies and thus to increase the efficacy and safety of the administered antibodies. The use of fully human antibodies can be expected to provide a substantial advantage in the treatment of chronic and recurring human diseases, such as cancer, which require repeated antibody administrations.

One approach toward the goal of producing fully human antibodies was to engineer mouse strains deficient in mouse antibody production to harbor large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human monoclonal antibodies with the desired specificity could be readily produced and selected.

This general strategy was demonstrated in connection with the generation of the first “XenoMouseT” strains as published in 1994. See Green et al., 1994, Nature Genetics, 7:13-21. The XenoMouse strains were engineered with yeast artificial chromosomes (YACS) containing 245 kb and 10 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human monoclonal antibodies. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through the use of megabase-sized, germline configuration YAC fragments of the human heavy chain loci and kappa light chain loci, respectively, to produce XenoMouse mice. See Mendez et al., 1997, Nature Genetics, 15:146-156; Green and Jakobovits, 1998, J. Exp. Med., 188:483-495; and Green, 1999, Journal of Immunological Methods, 231:11-23, the disclosures of which are hereby incorporated herein by reference.

Human anti-mouse antibody (HAMA) responses have led the industry to prepare chimeric or otherwise humanized antibodies. While chimeric antibodies typically are comprised of a human constant region and a murine variable region, it is expected that certain human anti-chimeric antibody (HACA) responses will be observed, particularly in treatments involving chronic or multi-dose utilizations of the antibody. Thus, it is desirable to provide fully human antibodies against the NF-κB pathway-associated polypeptides or peptides in order to vitiate concerns and/or effects of HAMA or HACA responses.

Polypeptide antibodies of the invention can be chemically synthesized or produced through the use of recombinant expression systems. Accordingly, the invention further embraces polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, an antibody that specifically binds to the NF-κB pathway-associated polypeptides having the amino acid sequences shown in Tables 1-6 (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 445, 447, 449, 451, 453, 455, 457, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 531, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 613, 615, 617, 619, 621, 623, 625, 627, 629, 631, 633, 635, 637, 640, 642, 644, 646, 648, 650, 652, 654, 656, 658, 660, 662, 664, 666, 668, 670, 672, 674, 676, 750, 752, 754, 756, 758, 760, 762, 764, 766, 768, 770, 772, 774, 776, 778 & 780).

Polynucleotides can be obtained, and the nucleotide sequence of the polynucleotides determined, by any method known in the art. For example, if the nucleotide sequence of the antibody is known, a polynucleotide encoding the antibody can be assembled from chemically synthesized oligonucleotides (e.g., as described in Kutmeier et al., 1994, BioTechniques, 17:242), which, briefly, involves the synthesis of overlapping oligonucleotides containing portions of the sequence encoding the antibody, the annealing and ligating of those oligonucleotides, and then the amplification of the ligated oligonucleotides by PCR.

Alternatively, a polynucleotide encoding an antibody can be generated from nucleic acid from a suitable source. If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library, or a cDNA library generated from, (or a nucleic acid, preferably poly A+ RNA, isolated from), any tissue or cells expressing the antibody, such as hybridoma cells selected to express an antibody of the invention by PCR amplification using synthetic primers hybridizable to the 3′ and 5′ ends of the sequence. Alternatively, cloning using an oligonucleotide probe specific for the particular gene sequence to identify, e.g., a cDNA clone from a cDNA library that encodes the antibody can be employed. Amplified nucleic acids generated by PCR can then be cloned into replicable cloning vectors using any method well known in the art.

Once the nucleotide sequence and corresponding amino acid sequence of the antibody are determined, the nucleotide sequence of the antibody can be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2nd Ed., Cold Spring Harbor Laboratory, Cold Spring Harbor, N.Y.; and Ausubel et al., eds., 1998, Current Protocols in Molecular Biology, John Wiley & Sons, N.Y., which are both incorporated by reference herein in their entireties), to generate antibodies having a different amino acid sequence, for example, to create amino acid substitutions, deletions, and/or insertions.

In a specific embodiment, the amino acid sequence of the heavy and/or light chain variable domains can be inspected to identify the sequences of the CDRs by methods that are well known in the art, e.g., by comparison to known amino acid sequences of other heavy and light chain variable regions, to determine the regions of sequence hypervariability. Using routine recombinant DNA techniques, one or more of the CDRs can be inserted within framework regions, e.g., into human framework regions, to humanize a non-human antibody, as described supra. The framework regions can be naturally occurring or consensus framework regions, and preferably, are human framework regions (see, e.g., Chothia et al., 1998, J. Mol. Biol., 278:457-479 for a listing of human framework regions).

Preferably, the polynucleotide generated by the combination of the framework regions and CDRs encodes an antibody that specifically binds to the NF-κB pathway-associated polypeptides. Also preferably, as discussed supra, one or more amino acid substitutions can be made within the framework regions; such amino acid substitutions are performed with the goal of improving binding of the antibody to its antigen. In addition, such methods can be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and are within the skill of the art.

For some uses, such as for in vitro affinity maturation of an anti-NF-κB pathway-associated polypeptide antibody of the invention, it is useful to express the V_{H} and V_{L} domains of the Ig heavy and light chains of one or more antibodies of the invention as single chain antibodies, or Fab fragments, in a phage display library using phage display methods as described supra. For example, the cDNAs encoding the V_{H} and V_{L} domains of one or more antibodies of the invention can be expressed in all possible combinations using a phage display library, thereby allowing for the selection of V_{H}/V_{L} combinations that bind to the NF-κB pathway-associated polypeptides or peptides thereof with preferred binding characteristics such as improved affinity or improved off rates. In addition, V_{H} and V_{L} segments, particularly, the CDR regions of the V_{H} and V_{L} domains of one or more antibodies of the invention, can be mutated in vitro. Expression of V_{H} and V_{L} domains with “mutant” CDRs in a phage display library allows for the selection of V_{H}/V_{L} combinations that bind to the NF-κB pathway-associated polypeptides.

In phage display methods, functional antibody domains are displayed on the surface of phage particles which carry the polynucleotide sequences encoding them. In particular, DNA sequences encoding the V_{H} and V_{L} domains are amplified from animal cDNA libraries (e.g., human or murine cDNA libraries of lymphoid tissues) or from synthetic cDNA libraries. The DNA encoding the V_{H} and V_{L} domains are joined together by an scFv linker by PCR and cloned into a phagemid vector (e.g., p CANTAB 6 or pComb 3 HSS). The vector is introduced into E. coli via electroporation and the E. coli is infected with helper phage. Phage used in these methods are typically filamentous phage, including fd and M13, and the V_{H} and V_{L} domains are usually recombinantly fused either to the phage gene III or gene VIII. Phage expressing an antigen binding domain that binds to an antigen of interest (i.e., the NF-κB pathway-associated polypeptide or a fragment thereof) can be selected or identified with antigen, e.g., using labeled antigen or antigen bound or captured onto a solid surface or bead.

Recombinant expression of an anti-NF-κB pathway-associated polypeptide antibody of the invention, or a fragment, derivative, variant, or analog thereof (e.g., a heavy or light chain of an antibody, or a single chain antibody, of the invention) requires construction of an expression vector containing a polynucleotide that encodes the antibody. Once a polynucleotide encoding an anti-NF-κB pathway-associated polypeptide antibody molecule, or a heavy or light chain of an antibody, or portion thereof (preferably containing the heavy or light chain variable domain), of the invention has been obtained, the vector for the production of the antibody molecule can be produced by recombinant DNA technology using techniques well known in the art. Methods for preparing a protein by expressing a polynucleotide encoding an antibody are described herein. Methods that are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus embraces replicable vectors comprising a nucleotide sequence encoding an antibody molecule of the invention, or a heavy or light chain thereof, or a heavy or light chain variable domain, operably linked to a promoter. Such vectors can include the nucleotide sequence encoding the constant region of the antibody molecule (see, e.g., PCT publication WO 86/05807; PCT publication WO 89/01036; and U.S. Pat. No. 5,122,464) and the variable domain of the antibody can be cloned into such a vector for expression of the entire heavy or light chain.

Methods of constructing expression vectors; types of vectors; methods of transferring the expression vectors into host cells and culturing the cells to produce antibodies; use of selection markers and systems; and the like, involve conventional techniques, and have been described above with respect to NF-κB pathway-associated protein expression. Such methods and the like are equally applicable for recombinant immunoglobulin protein expression and the production of anti-NF-κB pathway-associated polypeptide antibodies.

As one of its aspects, the invention includes host cells containing a polynucleotide encoding an anti-NF-κB pathway-associated polypeptide antibody, or a heavy or light chain thereof, or a single chain antibody of the invention, operably linked to a heterologous promoter. In preferred aspects for the expression of double-chained antibodies, vectors encoding both the heavy and light chains may be co-expressed in the host cell for expression of the entire immunoglobulin molecule, as detailed below.

The expression levels of an antibody molecule can be increased by vector amplification (for a review, see Bebbington and Hentschel, “The use of vectors based on gene amplification for the expression of cloned genes in mammalian cells in DNA cloning”, Vol. 3. (Academic Press, New York, 1987). When a marker in the vector system expressing an antibody is amplifiable, an increase in the level of inhibitor present in the host cell culture increases the number of copies of the marker gene. Since the amplified region is associated with the antibody gene, production of the antibody will also increase (Crouse et al., 1983, Mol. Cell. Biol., 3:257).

Vectors which use glutamine synthase (GS) or DHFR as the selectable markers can be amplified in the presence of the drugs methionine sulphoximine or methotrexate, respectively. An advantage of glutamine synthase based vectors is the availability of cell lines (e.g., the murine myeloma cell line, NSO) that are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene.

Vectors that express glutamine synthase as the selectable marker include, but are not limited to, the pEE6 expression vector described in Stephens and Cockett, 1989, Nucl. Acids. Res., 17:7110. A glutamine synthase expression system and components thereof are detailed in PCT publications: W087/04462; W086/05807; W089/01036; W089/10404; and W091/06657 which are incorporated by reference herein in their entireties. In addition, glutamine synthase expression vectors that can be used in accordance with the present invention are commercially available from suppliers, including, for example, Lonza Biologics, Inc. (Portsmouth, N.H.). The expression and production of monoclonal antibodies using a GS expression system in murine myeloma cells is described in Bebbington et al., 1992, BioTechnology, 10:169 and in Biblia and Robinson, 1995, Biotechnol. Prog., 11:1, which are incorporated by reference herein in their entireties.

A host cell can be co-transfected with two expression vectors of the invention, the first vector encoding an Ig heavy chain derived polypeptide and the second vector encoding an Ig light chain derived polypeptide. The two vectors can contain identical selectable markers that enable equal expression of heavy and light chain polypeptides. Alternatively, a single vector can be used which encodes, and is capable of expressing, both the Ig heavy and light chain polypeptides. In such situations, the light chain should be placed before the heavy chain to avoid an excess of toxic free heavy chain (Proudfoot, 1986, Nature, 322:52; Kohler, 1980, Proc. Natl. Acad. Sci. USA, 77:2197). The coding sequences for the heavy and light chains can comprise cDNA or genomic DNA.

Once an antibody molecule against a NF-κB pathway-associated polypeptide of the invention has been produced by an animal, chemically synthesized, or recombinantly expressed, it can be purified by any method known in the art for the purification of an immunoglobulin or polypeptide molecule, for example, by chromatography (e.g., ion exchange, affinity, particularly by affmity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies that are recombinantly fused or chemically conjugated (including both covalently and non-covalently conjugated) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but can occur through linker sequences. The antibodies can be specific for NF-κB pathway-associated polypeptide antigens (or portions thereof, preferably at least 10, 20, 30, 40, 50, 60, 70, 80, 90 or 100 amino acids of the polypeptide). For example, antibodies can be used to target the NF-κB pathway-associated polypeptide to particular cell types, either in vitro or in vivo, by fusing or conjugating NF-κB pathway-associated polypeptide to antibodies specific for particular cell surface receptors.

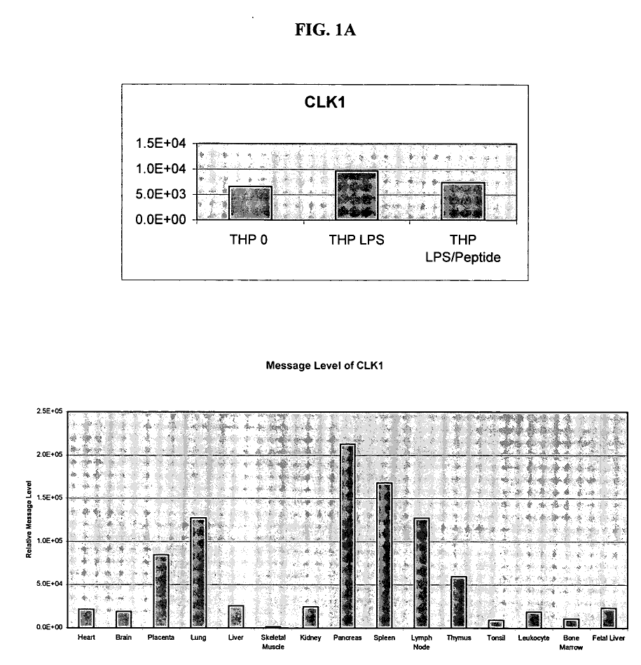

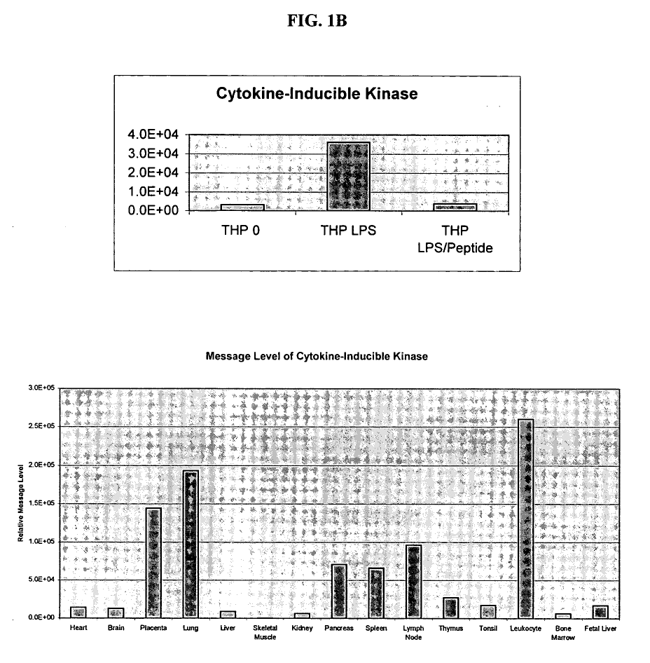

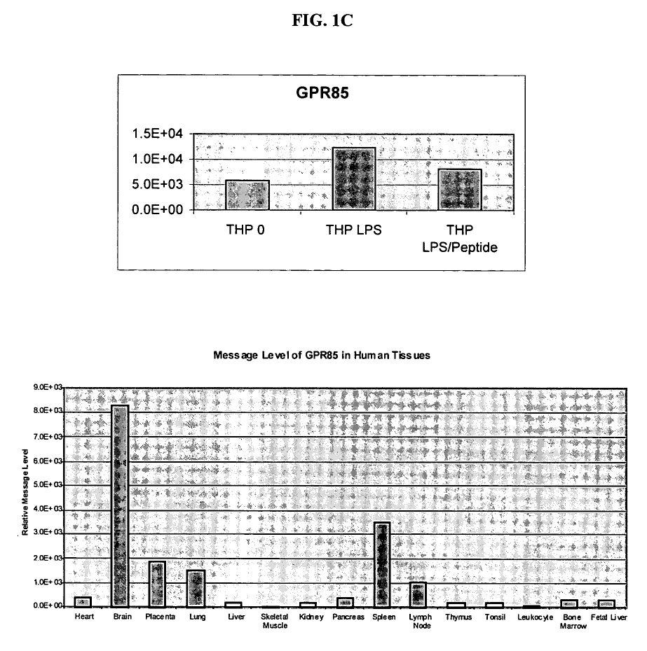

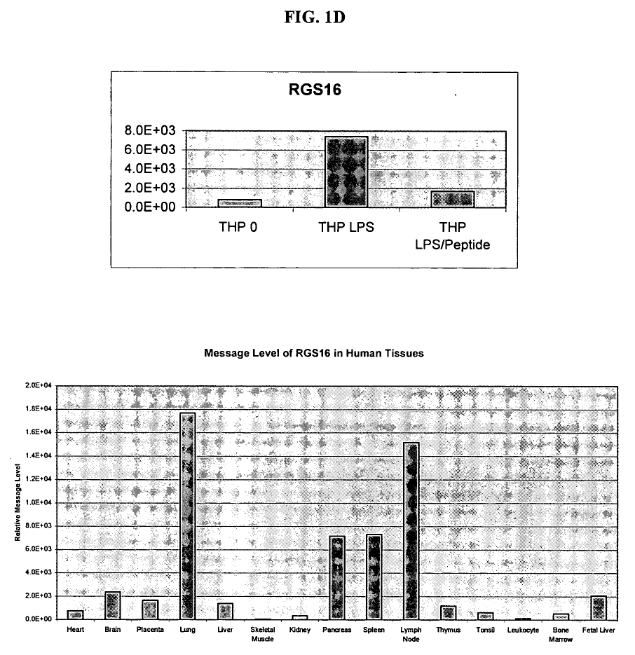

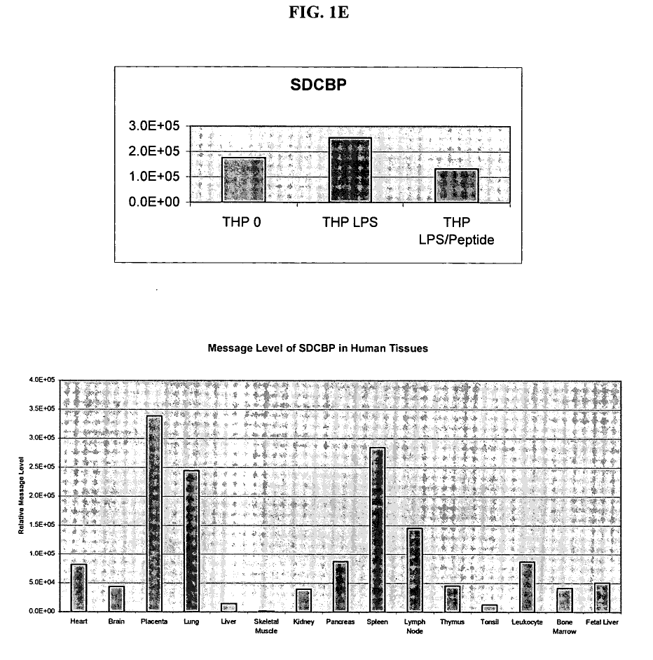

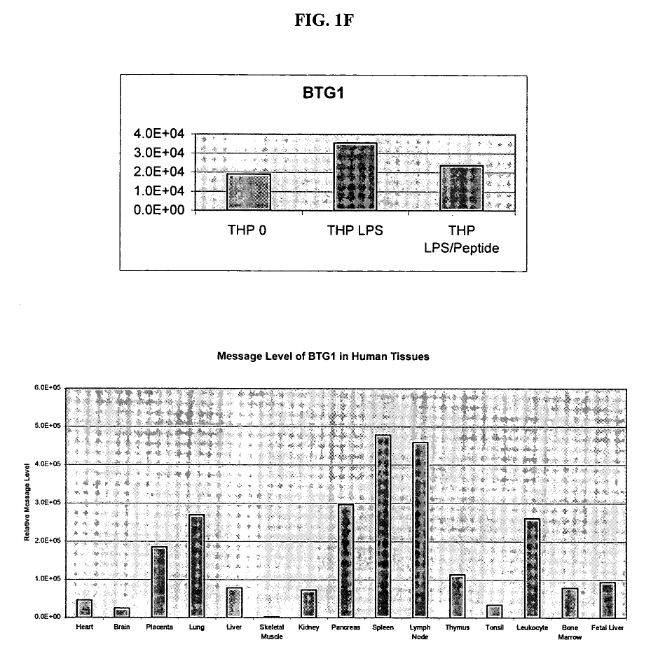

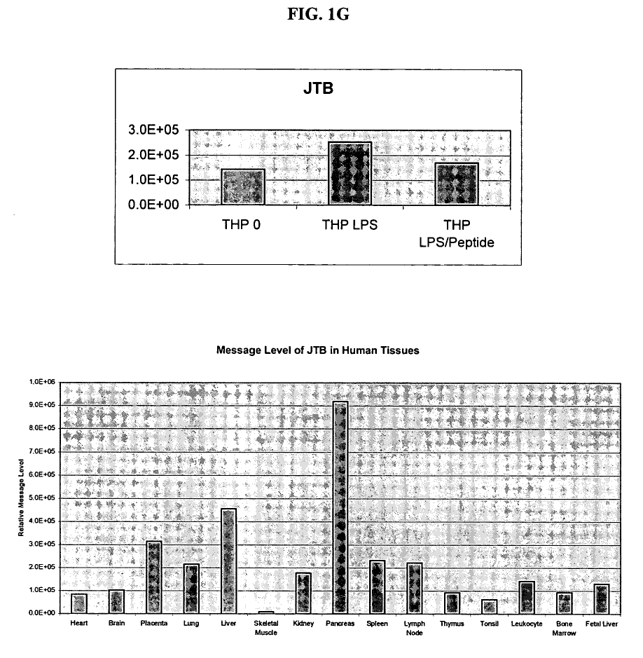

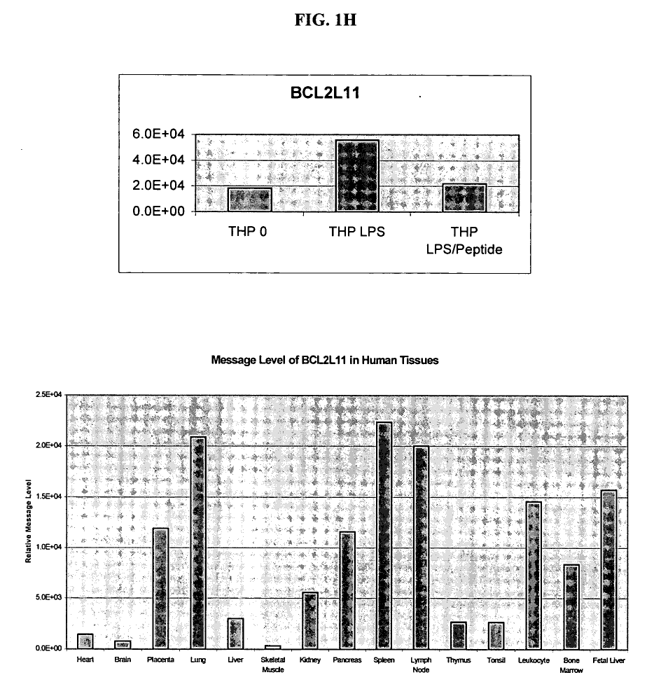

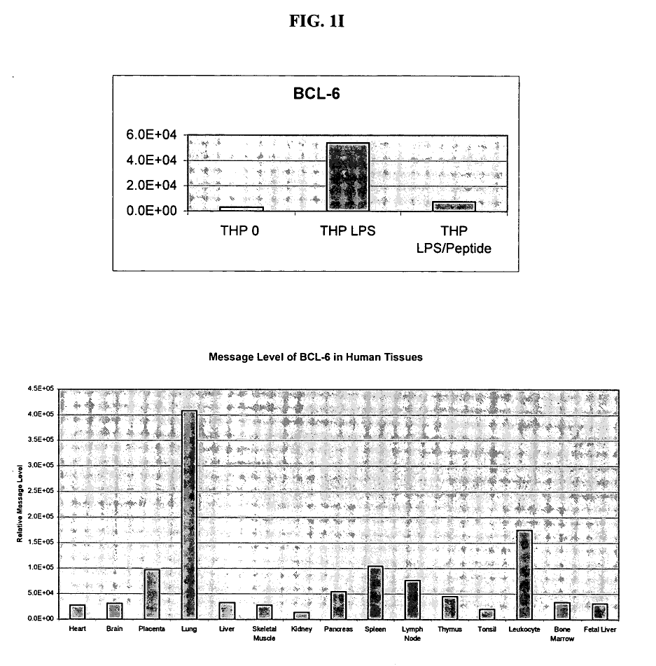

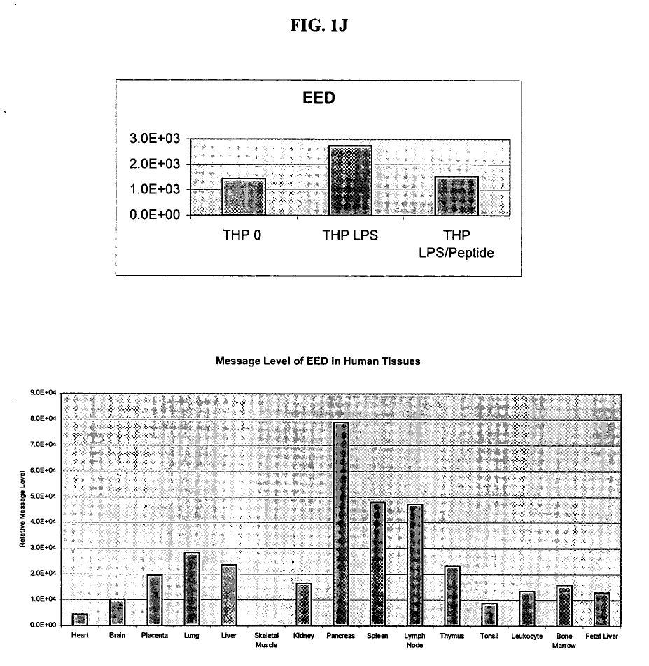

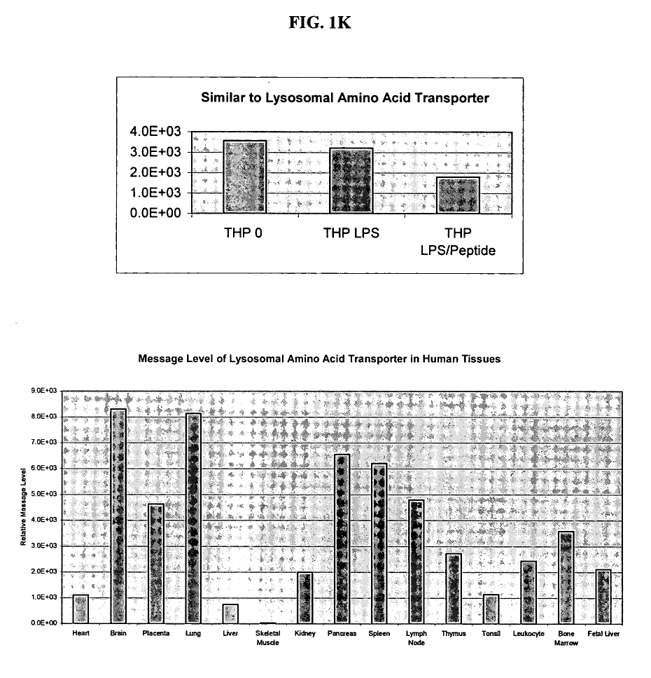

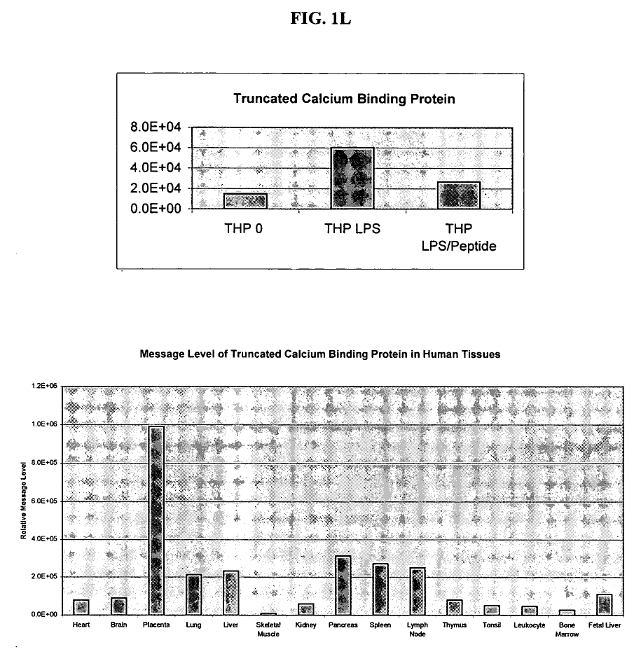

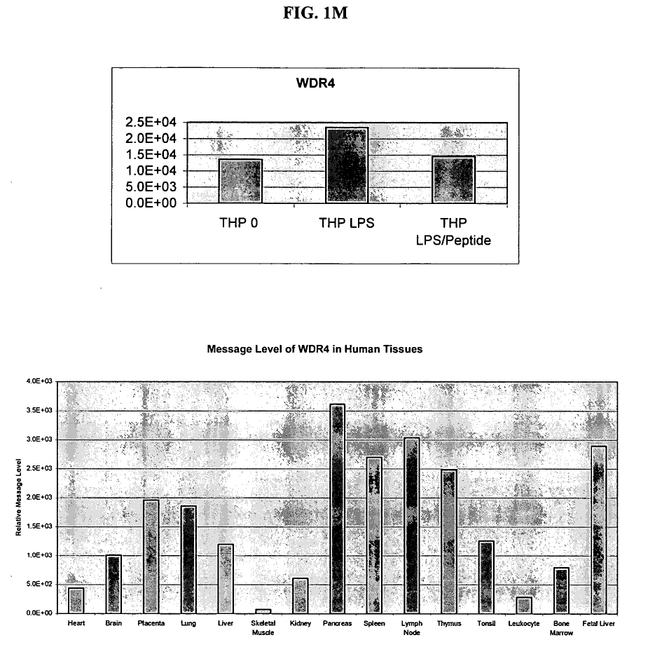

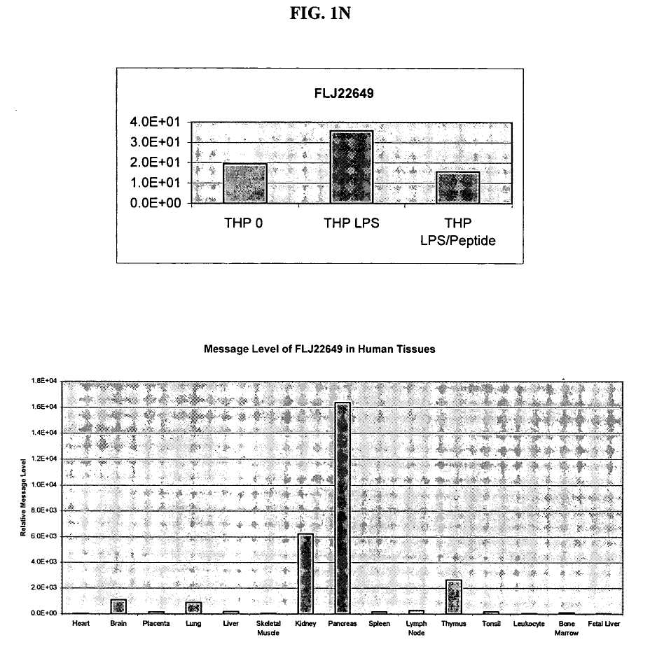

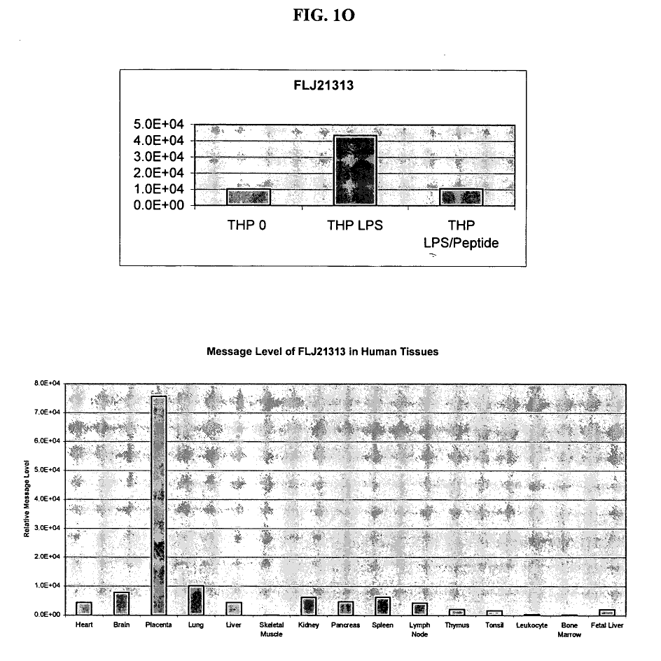

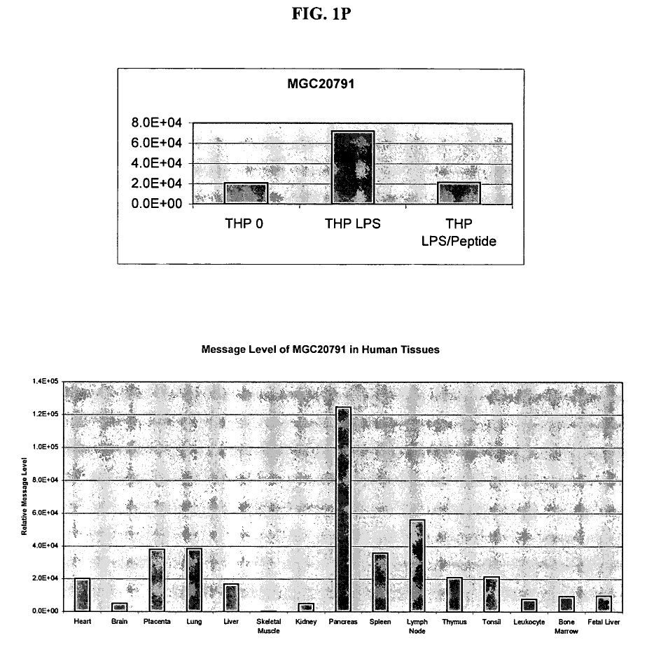

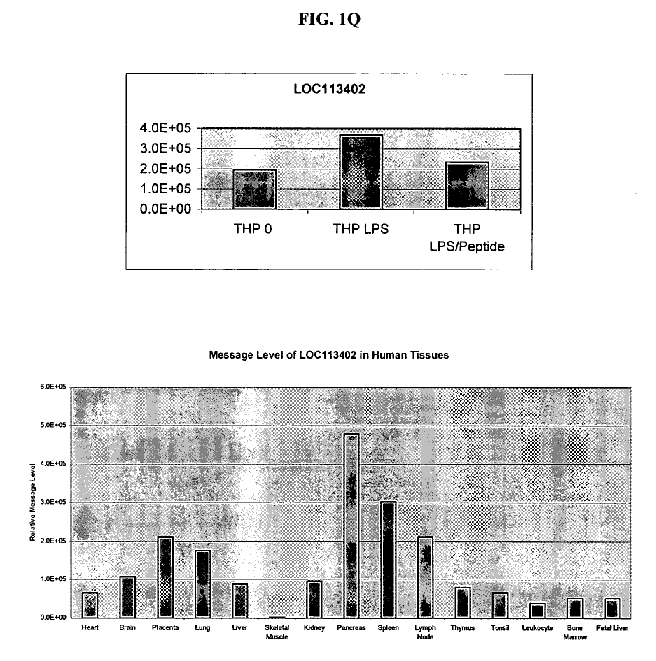

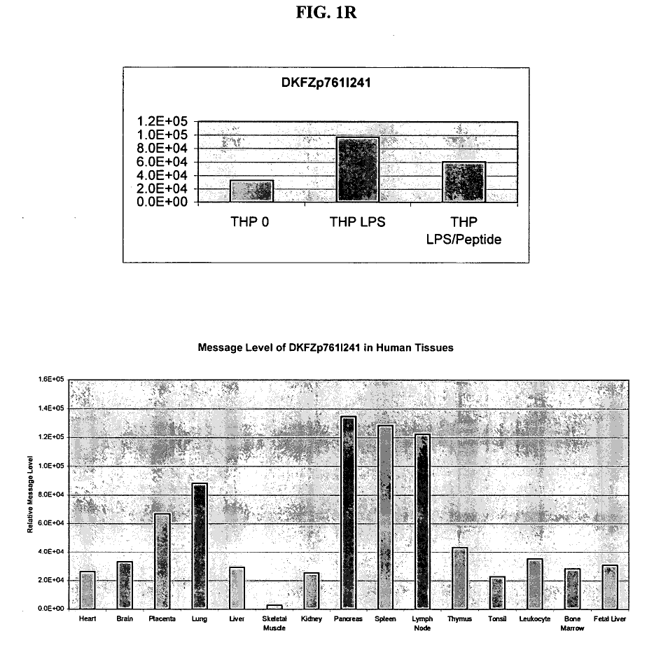

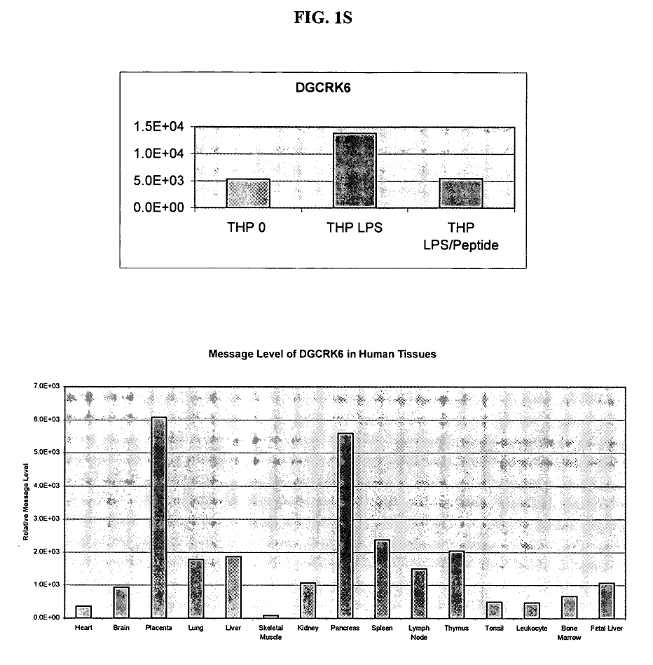

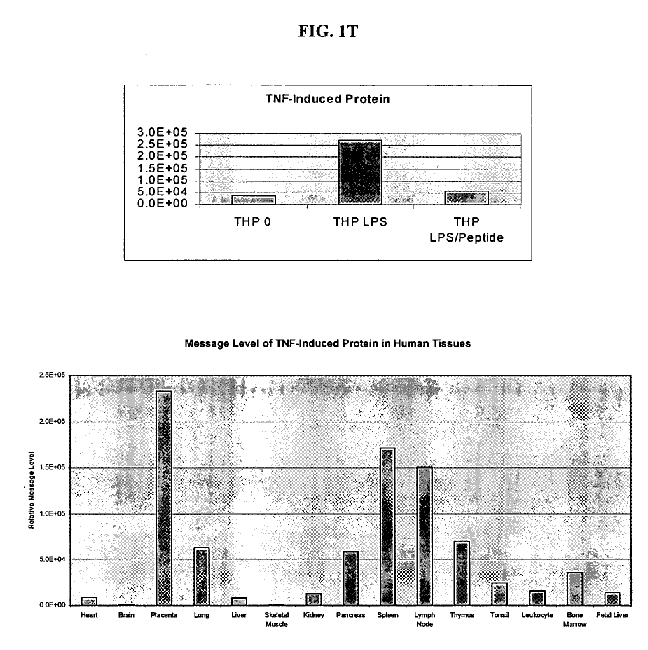

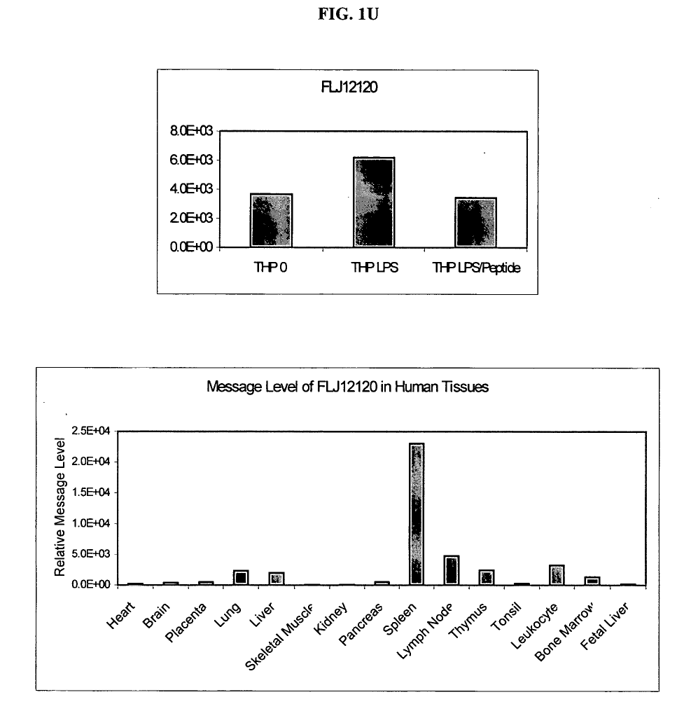

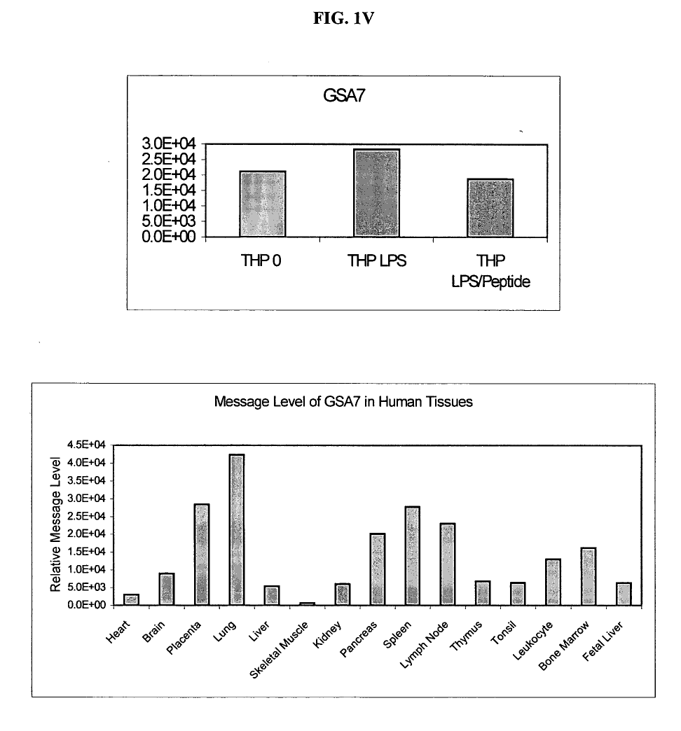

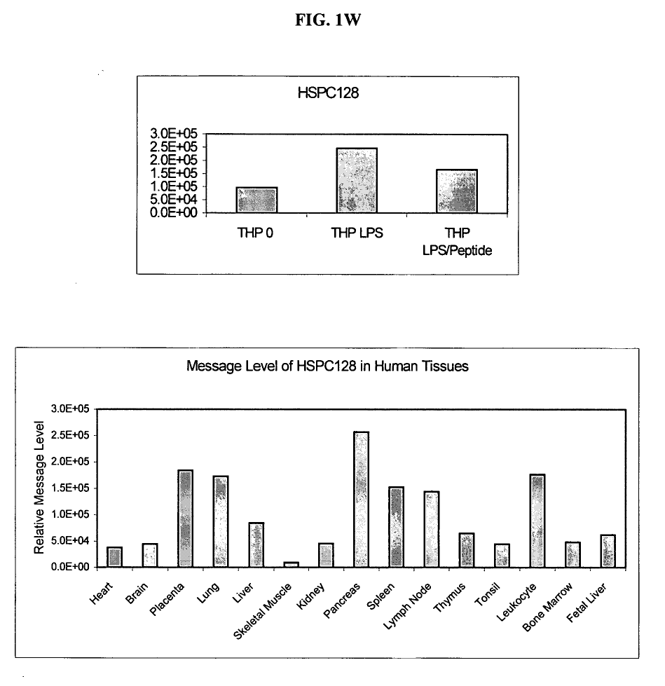

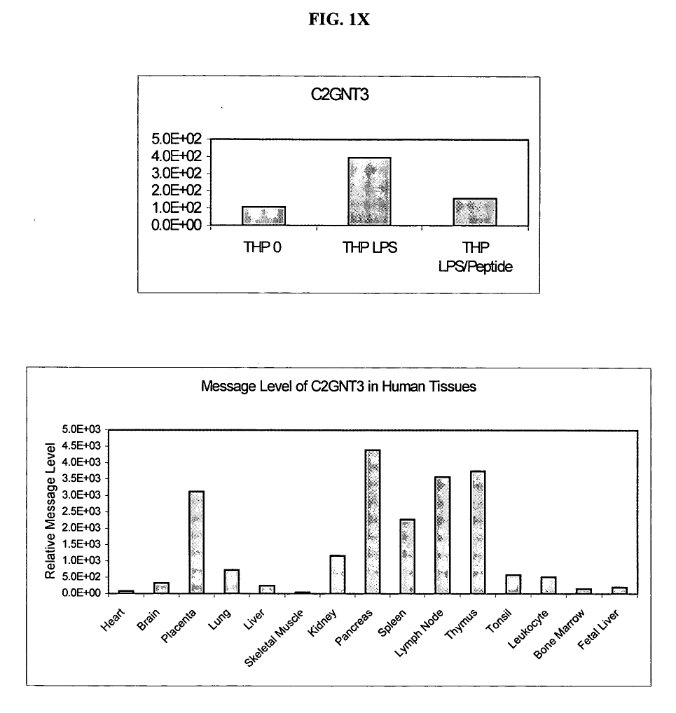

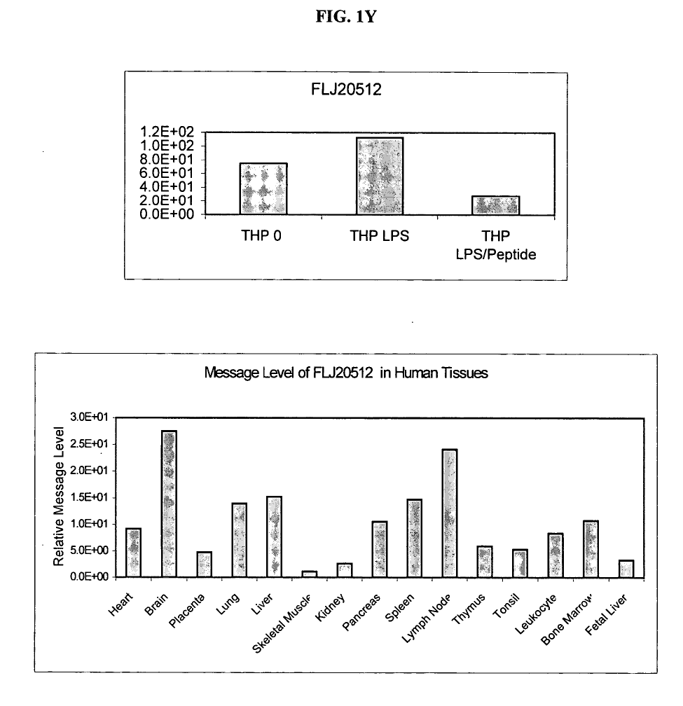

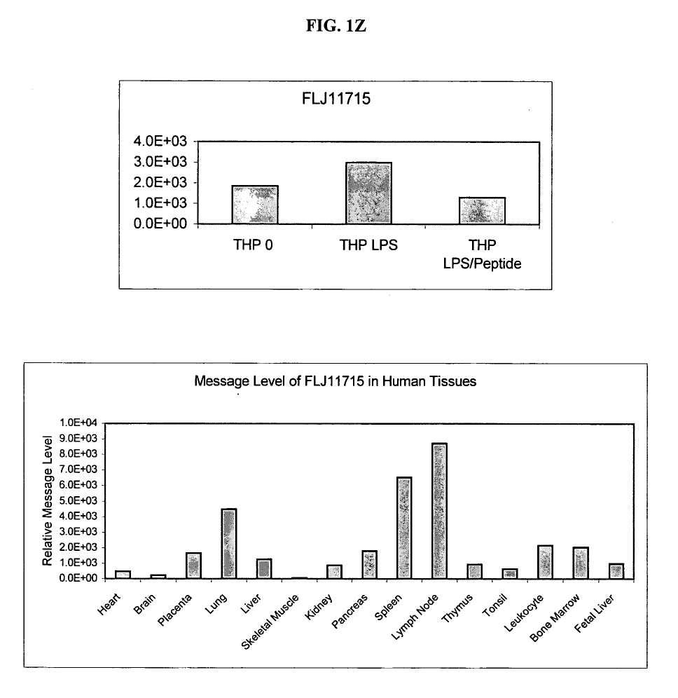

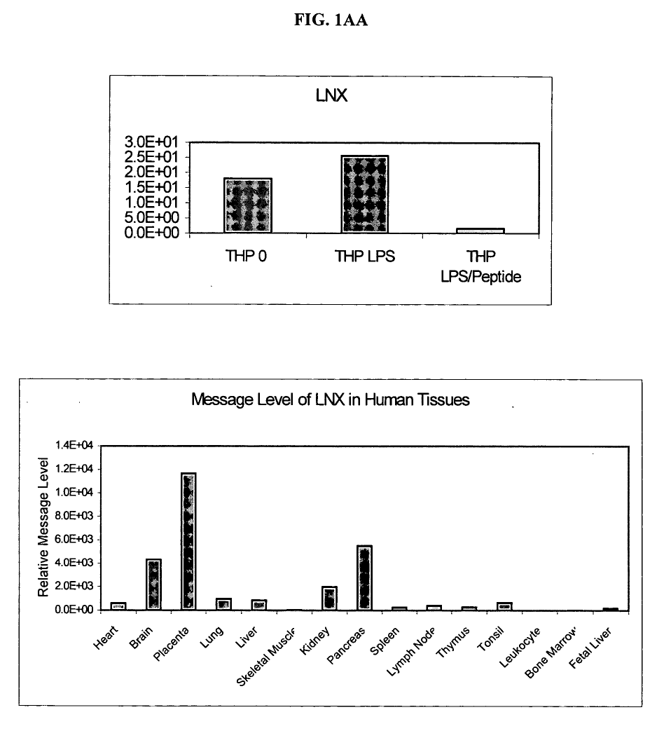

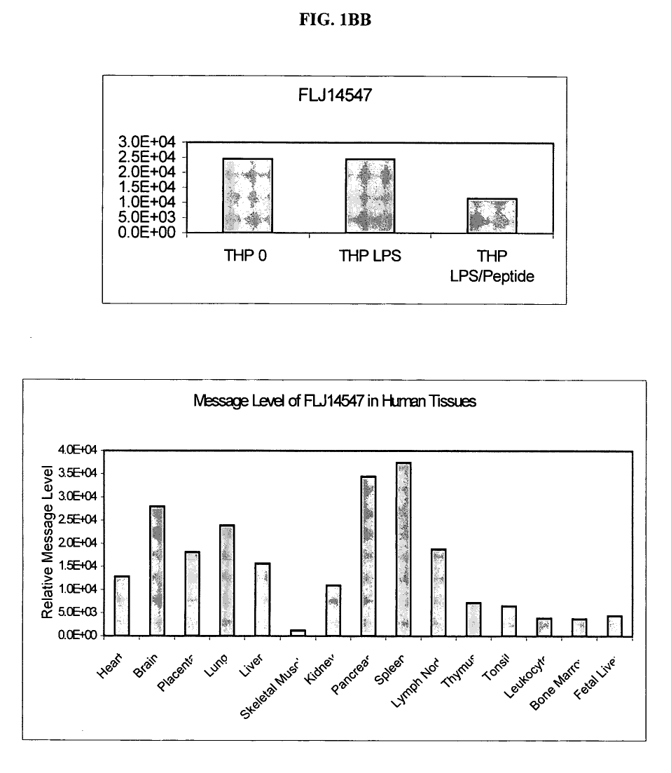

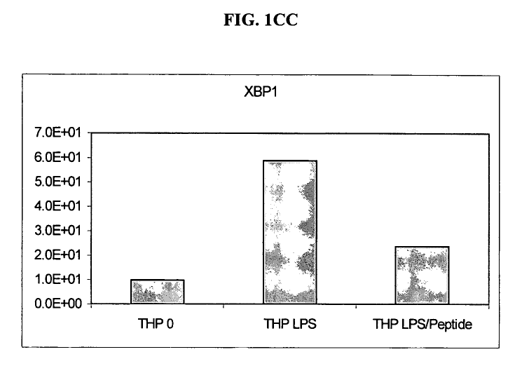

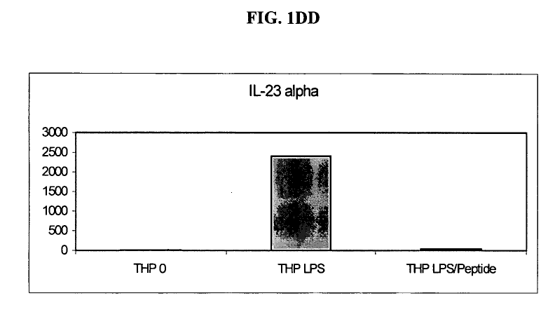

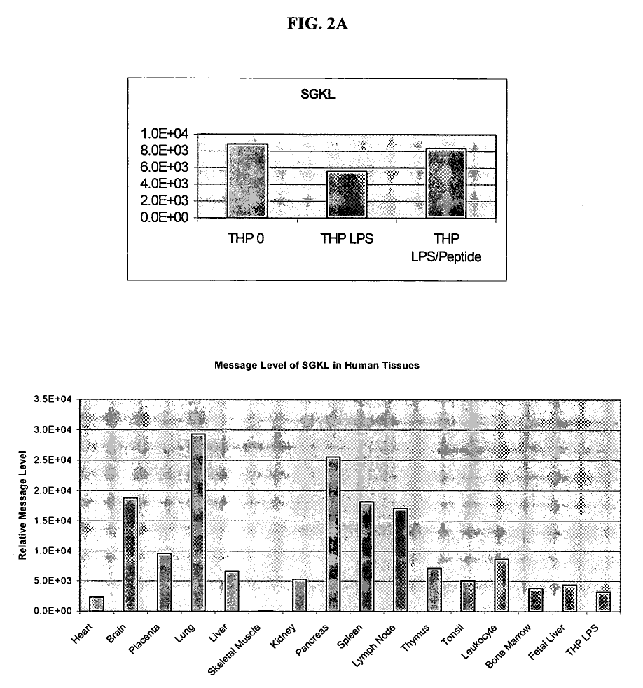

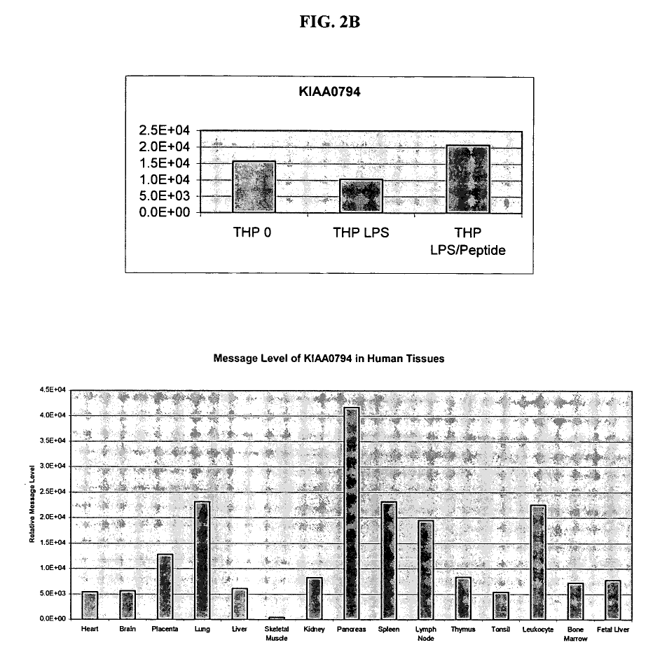

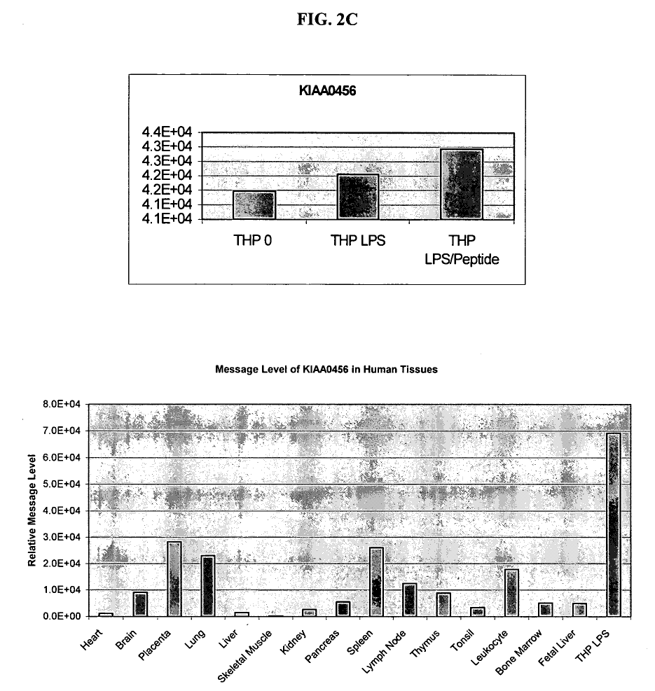

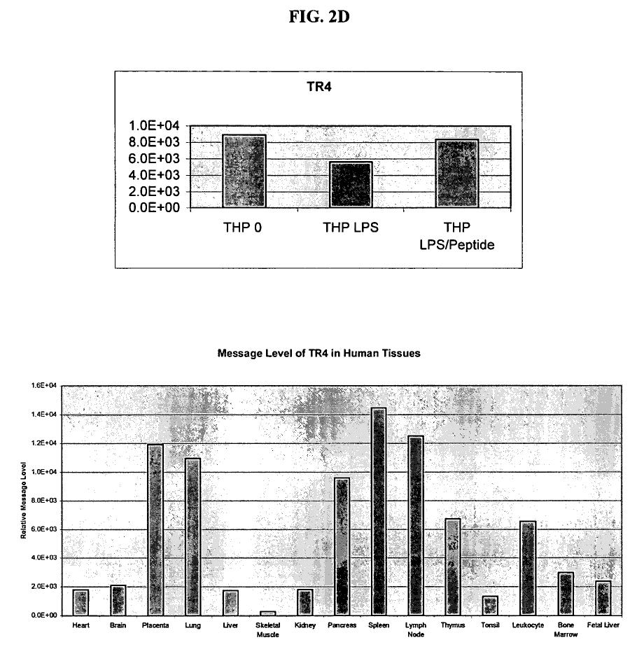

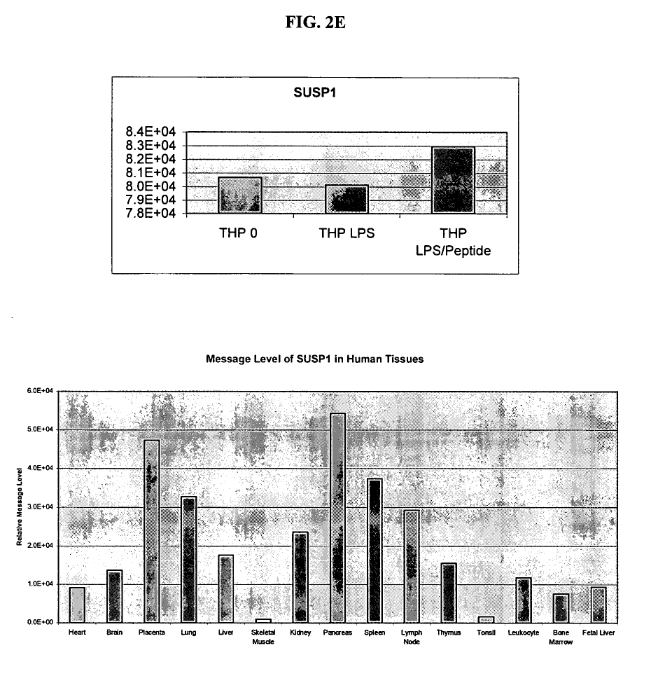

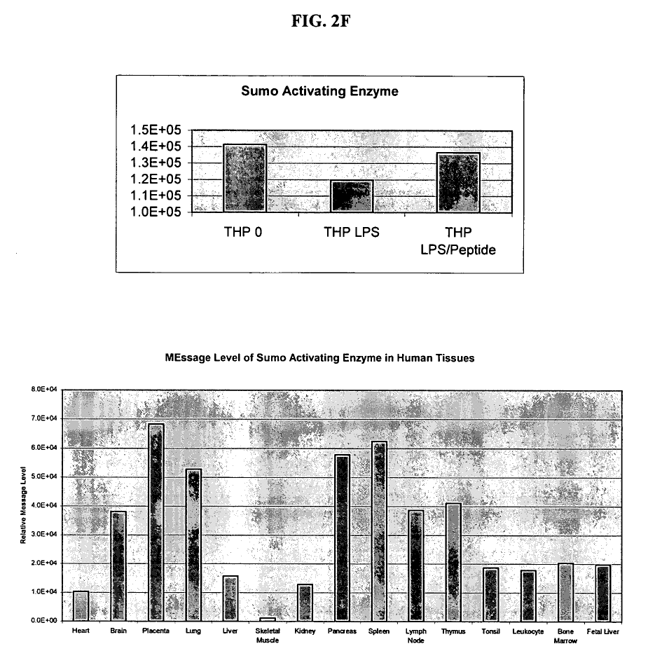

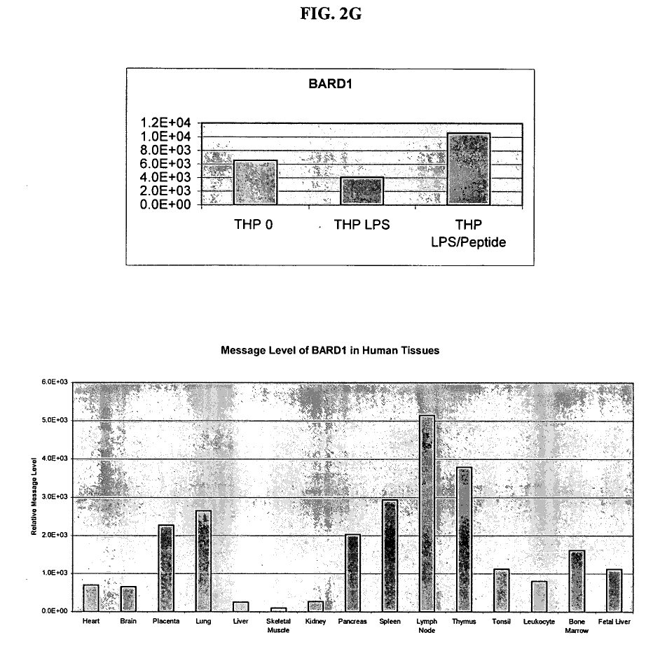

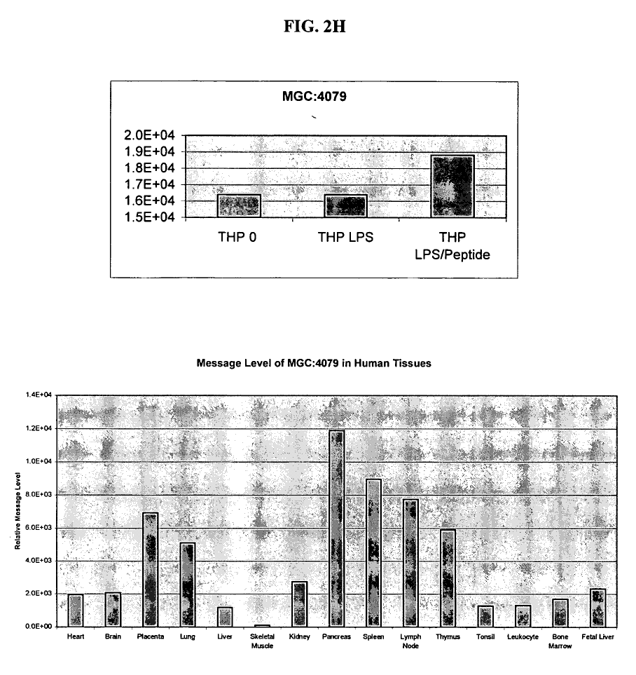

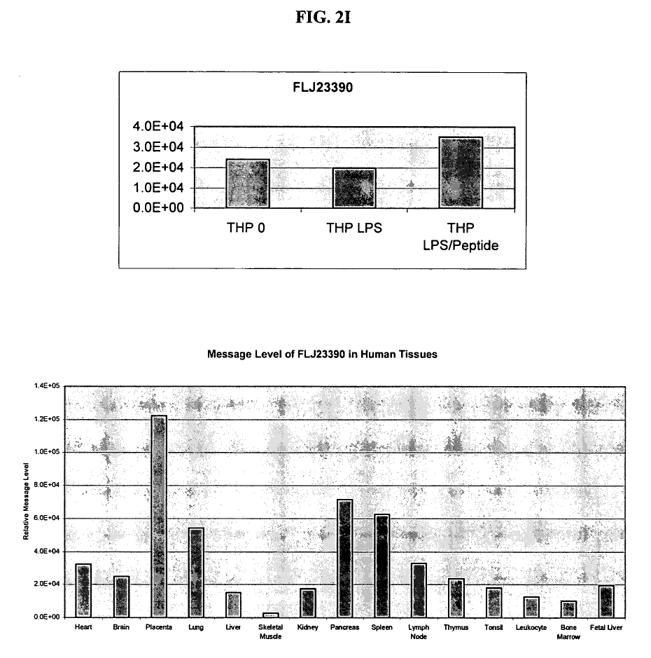

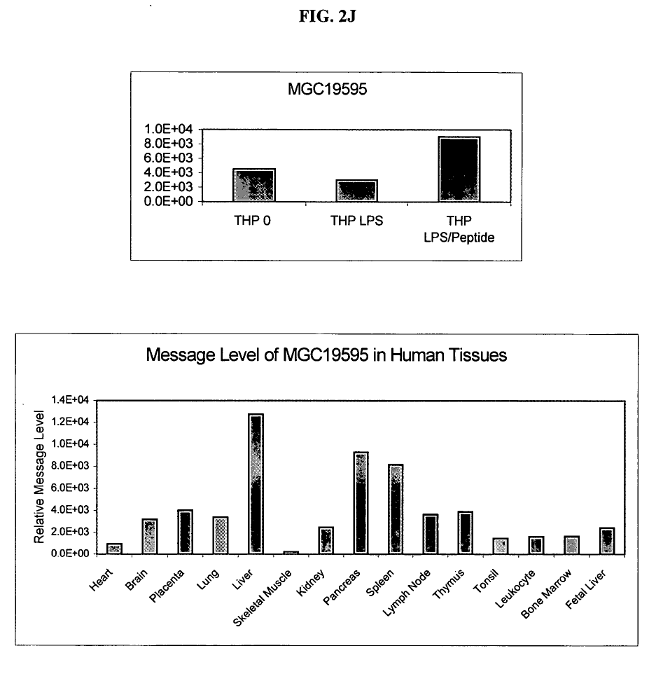

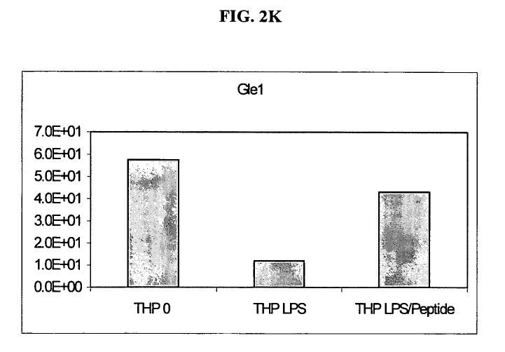

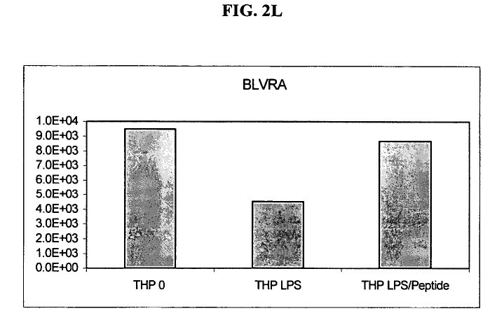

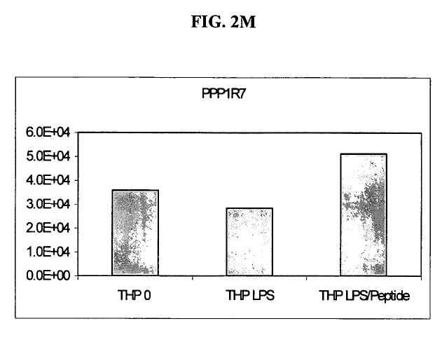

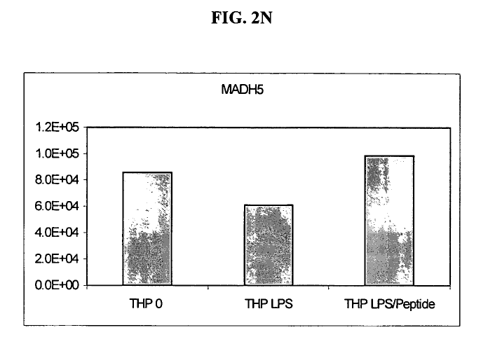

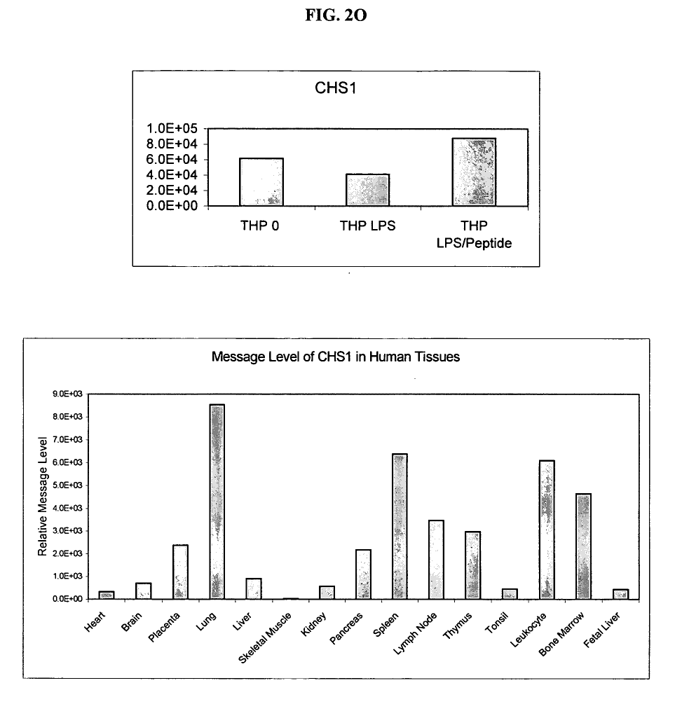

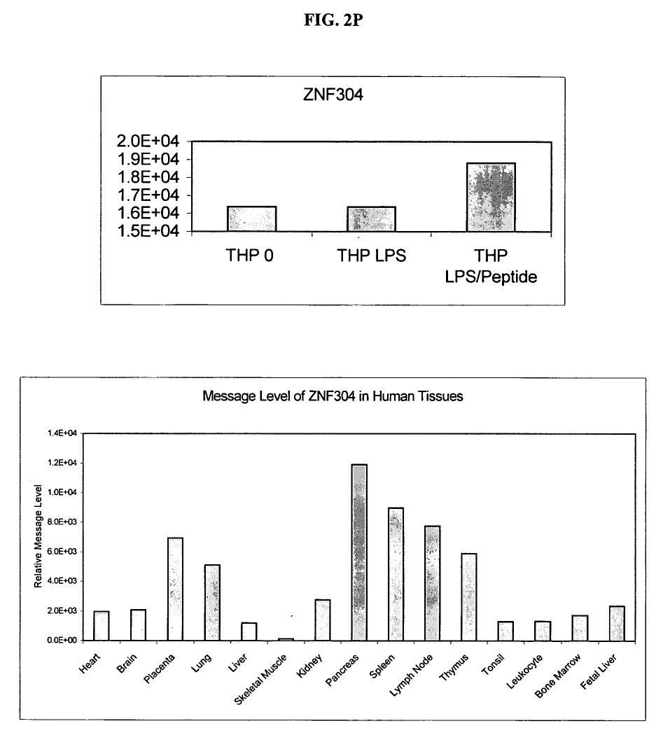

NF-κB pathway-associated polypeptides or antibodies raised against the NF-κB pathway-associated polypeptides of the present invention (including fragments or variants thereof) can be fused to either the N-terminal or C-terminal end of a heterologous protein (e.g., immunoglobulin Fc polypeptide or human serum albumin polypeptide). Antibodies of the invention can also be fused to albumin (including, but not limited to, recombinant human serum albumin (see, e.g., U.S. Pat. No. 5,876,969, issued Mar. 2, 1999; EP Patent 0 413 622; and U.S. Pat. No. 5,766,883, issued Jun. 16, 1998, incorporated herein by reference in their entirety), resulting in chimeric polypeptides. In a preferred aspect, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094, which is herein incorporated by reference in its entirety). In another preferred aspect, polypeptides and/or antibodies of the present invention (including fragments or variants thereof) are fused with polypeptide fragments comprising, or alternatively consisting of, amino acid residues 1-z of human serum albumnin, where z is an integer from 369 to 419, as described in U.S. Pat. No. 5,766,883 incorporated herein by reference in its entirety.

Polynucleotides encoding NF-κB pathway-associated polypeptide fusion proteins and antibodies thereto are also encompassed by the invention. Such fusion proteins can, for example, facilitate purification and can increase half-life in vivo. Antibodies fused or conjugated to the polypeptides of the present invention can also be used in in vitro immunoassays and purification methods using methods known in the art. See, e.g., Harbor et al., supra, and PCT publication WO 93/21232; EP 439, 095; Naramura et al., 1994, Immunol. Lett., 39:91-99; U.S. Pat. No. 5,474,981; Gillies et al., 1992, Proc. Natl. Acad. Sci. USA, 89:1428-1432; Fell et al., 1991, J. Immunol., 146:2446-2452, which are incorporated by reference herein in their entireties. For guidance, chimeric proteins having the first two domains of the human CD4 polypeptide and various domains of the constant regions of the heavy or light chains of mammalian immunoglobulins have been described. (EP 394,827; Traunecker et al., 1988, Nature, 331:84-86). NF-κB pathway-associated polypeptide or peptide fused or conjugated to an antibody, or portion thereof, having disulfide-linked dimeric structures (due to the IgG), for example, can also be more efficient in binding and neutralizing other molecules, than the monomeric secreted protein or protein fragment alone. (Fountoulakis et al., 1995, J. Biochem., 270:3958-3964).

The present invention further includes compositions comprising the NF-κpathway-associated polypeptides or peptides thereof fused or conjugated to antibody domains other than the variable region domain. For example, the polypeptides of the present invention can be fused or conjugated to an antibody Fc region, or portion thereof. The antibody portion fused to a polypeptide of the present invention can comprise the constant region, hinge region, CH1 domain, CH2 domain, CH3 domain, or any combination of whole domains or portions thereof. The polypeptides can also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating polypeptides to antibody portions are known in the art. (See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., 1991, Proc. Natl. Acad Sci. USA, 88:10535-10539; Zheng et al., 1995, J. Immunol., 154:5590-5600; and Vil et al., Proc. Natl. Acad. Sci. USA, 89:11337-11341, which are hereby incorporated by reference herein in their entireties).

In many cases, the Fc portion in a fusion protein is beneficial in therapy, diagnosis, and/or screening methods, and thus can result in, for example, improved pharmacokinetic properties. (EP A 232, 262). In drug discovery, for example, human proteins, such as hIL-5, have been fused with Fc portions for the purpose of high-throughput screening assays to identify antagonists of hIL-5. (See, Bennett et al., 1995, J. Molecular Recognition, 8:52-58; and Johanson et al., 1995, J. Biol. Chem., 270:9459-9471). Alternatively, deleting the Fc portion after the fusion protein has been expressed, detected, and purified, may be desired. For example, the Fc portion may hinder therapy and diagnosis if the fusion protein is used as an antigen for immunizations.

Moreover, according to this invention, anti-NF-κB pathway-associated antibodies or fragments thereof can be fused to marker sequences, such as a peptide, to facilitate their purification. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., 1989, Proc. Natl. Acad. Sci. USA, 86:821-824, for instance, hexa histidine provides for convenient purification of the fusion protein. Other peptide tags useful for purification include, but are not limited to, the “HA” tag and the Flag tag, as previously described herein.

The present invention further encompasses antibodies or fragments thereof conjugated to a diagnostic or therapeutic agent. The antibodies can be used diagnostically, for example, to monitor the development or progression of a tumor as part of a clinical testing procedure, or to determine the efficacy of a given treatment regimen. Detection can be facilitated by coupling the antibody to a detectable substance. Nonlimiting examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or fragment thereof) or indirectly, through an intermediate (such as, for example, a linker as known in the art) using techniques known in the art. (See, for example, U.S. Pat. No. 4,741,900 for metal ions which can be conjugated to antibodies for use as diagnostics according to the present invention).

Nonlimiting examples of suitable detectable enzymes include horseradish peroxidase, alkaline phosphatase, beta-galactosidase, or acetylcholinesterase; Nonlimiting examples of suitable prosthetic group complexes include streptavidin/biotin and avidinibiotin; nonlimiting examples of suitable fluorescent materials include umbelliferone, fluorescein, fluorescein isothiocyanate, rhodamine, dichlorotriazinylamine fluorescein, dansyl chloride or phycoerythrin; a nonlimiting example of a luminescent material includes luminol; nonlimiting examples of bioluminescent materials include luciferase, luciferin, and aequorin; and nonlimiting examples of suitable radioactive material include iodine (¹²⁵I, ¹³¹I), carbon (¹⁴C), sulfur (3 sus), tritium (³H), indium (¹¹¹In and other radioactive isotopes of inidium), technetium (⁹⁹Tc, ^{99m}Tc), thallium (20′Ti), gallium (⁶⁸Ga, ⁶⁷Ga), palladium (¹⁰³Pd), molybdenum (⁹⁹Mo), xenon (¹³³Xe), fluorine (¹⁹F), ¹⁵³Sm, ¹⁷⁷Lu, radioactive Gd, radioactive Pm, radioactive La, radioactive Yb, ¹⁶⁶Ho,⁹⁰Y, radioactive Sc, radioactive Re, radioactive Re, ¹⁴²Pr, ¹⁰⁵Rh, and ⁹⁷Ru.

In specific aspects, the NF-κB pathway-associated protein or a peptide portion thereof is attached to macrocyclic chelators useful for conjugating radiometal ions, including, but not limited to, ¹¹¹In, ¹⁷⁷Lu, ⁹⁰Y, ¹⁶⁶Ho, and ¹⁵³Sm, to polypeptides. In a preferred aspect, the radiometal ion associated with the macrocyclic chelators attached to the NF-κB pathway-associated protein or peptide is ¹¹¹In. In another preferred aspect, the radiometal ion associated with the macrocyclic chelator attached to the NF-κB pathway-associated protein or peptide is ⁹⁰Y. In specific aspects, the macrocyclic chelator is 1,4,7,10-tetraazacyclododecane-N,N′,N″,N′″-tetraacetic acid (DOTA). In other specific aspects, the DOTA is attached to the NF-κB pathway-associated protein or peptide via a linker molecule.

Examples of linker molecules useful for conjugating DOTA to a polypeptide are commonly known in the art. (See, for example, DeNardo et al., 1998, Clin. Cancer Res., 4(10):2483-90; Peterson et al., 1999, Bioconjug. Chem., 10(4):553-557; and Zimmerman et al, 1999, Nucl. Med. Biol., 26(8):943-950, which are hereby incorporated by reference in their entirety. In addition, U.S. Pat. Nos. 5,652,361 and 5,756,065, which disclose chelating agents that can be conjugated to antibodies and methods for making and using them, are hereby incorporated by reference in their entireties. Although U.S. Pat. Nos. 5,652,361 and 5,756,065 focus on conjugating chelating agents to antibodies, one skilled in the art can readily adapt the methods disclosed therein in order to conjugate chelating agents to other polypeptides. Antibodies can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating therapeutic moieties to antibodies are well known, see, e.g., Arnon et al., “Monoclonal Antibodies For Immunotargeting Of Drugs In Cancer Therapy”, In: Monoclonal Antibodies And Cancer Therapy, Reisfeld et al. (eds.), pp. 243-56, Alan R. Liss, Inc., 1985; Hellstrom et al., “Antibodies For Drug Delivery”, In: Controlled Drug Delivery (2nd Ed.), Robinson et al. (eds.), pp. 623-53, Marcel Deldcer, Inc., 1987; Thorpe, “Antibody Carriers Of Cytotoxic Agents In Cancer Therapy: A Review”, In: Monoclonal Antibodies '84: Biological And Clinical Applications, Pinchera et al. (eds.), pp. 475-506, 1985; “Analysis, Results, And Future Prospective Of The Therapeutic Use Of Radiolabeled Antibody In Cancer Therapy”, In: Monoclonal Antibodies For Cancer Detection And Therapy, Baldwin et al. (eds.), pp. 303-316, Academic Press, 1985; and. Thorpe et al., 1982, “The Preparation And Cytotoxic Properties Of Antibody-Toxin Conjugates”, Immunol. Rev., 62:119-158. Alternatively, an antibody can be conjugated to a second antibody to form an antibody heteroconjugate, e.g., as described in U.S. Pat. No. 4,676,980 to Segal, which is incorporated herein by reference in its entirety. An antibody, i.e., an antibody specific for NF-κB pathway-associated protein, with or without a therapeutic moiety conjugated to it, and administered alone or in combination with cytotoxic factor(s) and/or cytokine(s), can be used as a therapeutic.

The antibodies of the invention can be utilized for immunophenotyping of cell lines and biological samples. The translation product of the NF-κB pathway-associated protein-encoding nucleic acid can be useful as cell specific marker(s), or more specifically, as cellular marker(s) that are differentially expressed at various stages of differentiation and/or maturation of particular cell types (e.g., in particular tissues). Monoclonal antibodies directed against a specific epitope, or combination of epitopes, allow for the screening of cellular populations expressing the marker. Various techniques utilizing monoclonal antibodies can be employed to screen for cellular populations expressing the marker(s), including magnetic separation using antibody-coated magnetic beads, “panning” with antibody(ies) attached to a solid matrix (i.e., tissue culture plate), and flow cytometry (See, e.g., U.S. Pat. No. 5,985,660; and Morrison et al., 1999, Cell, 96:737-749). The above techniques allow for the screening of particular populations of cells, such as might be found with cancers or malignancies (i.e., minimal residual disease (MRD), for example, in lung cancer patients) and “non-self” cells in transplantations to prevent graft-versus-host disease (GVHD).

Anti-NF-κB pathway-associated protein antibodies according to this invention can be assayed for immunospecific binding by any method known in the art. The immunoassays which can be used include, but are not limited to, competitive and non-competitive assay systems using techniques such as BIAcore analysis, FACS (Fluorescence Activated Cell Sorter) analysis, immunofluorescence, immunocytochemistry, Western blots, radioimmunoassays, ELISA (enzyme linked immunosorbent assays), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few. Such assays are routine and well known and practiced in the art (see, e.g., Ausubel et al, eds, 1994, Current Protocols in Molecular Biology, Vol. 1, John Wiley & Sons, Inc., New York, which is incorporated by reference herein in its entirety). Nonlimiting, exemplary immunoassays are described briefly below.

Immunoprecipitation protocols generally comprise lysing a population of cells in a lysis buffer such as RIPA buffer (i.e., 1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate); adding the antibody of interest to the cell lysate; incubating for a period of time (e.g., 1 to 4 hours) at 4° C.; adding protein A and/or protein G sepharose beads to the cell lysate; incubating for about 60 minutes or more at 4° C.; washing the beads in lysis buffer; and resuspending the beads in SDS/sample buffer. The ability of the antibody of interest to immunoprecipitate a particular antigen can be assessed by, for example, Western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols, see, e.g., Ausubel et al, eds, 1994, Current Protocols in Molecular Biology, Vol. 1, John Wiley & Sons, Inc., New York, at 10.16.1.

Western blot analysis generally comprises preparing protein samples; electrophoresis of the protein samples in a polyacrylamide gel (e.g., 8%-20% SDS PAGE depending on the molecular weight of the antigen); transferring the protein sample from the polyacrylamide gel to a solid support membrane such as nitrocellulose, PVDF or nylon; blocking the membrane in blocking solution (e.g., PBS with 3% BSA or nonfat milk); washing the membrane in washing buffer (e.g., PBS-Tween 20); blocking the membrane with primary antibody (the antibody of interest) diluted in blocking buffer; washing the membrane in washing buffer; blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer; washing the membrane in wash buffer; and detecting the presence of the antigen. One of skill in the art would be knowledgeable as to the parameters that can be modified to increase the signal detected and to reduce the background noise. For further discussion regarding Western blot protocols, see, e.g., Ausubel et al, eds, 1994, Current Protocols in Molecular Biology, Vol. 1, John Wiley & Sons, Inc., New York, at 10.8.1.

ELISAs comprise preparing antigen; coating the wells of a 96 well microtiter plate with antigen; adding to the wells the antibody of interest conjugated to a detectable compound such as an enzymatic substrate (e.g., horseradish peroxidase or alkaline phosphatase); incubating for a period of time; and detecting the presence of the antigen. In ELISAs, the antibody of interest does not have to be conjugated to a detectable compound; instead, a second antibody (which recognizes the antibody of interest) conjugated to a detectable compound can be added to the wells. Further, instead of coating the wells with antigen, the antibody can be first coated onto the well. In this case, a second antibody conjugated to a detectable compound can be added to the antibody-coated wells following the addition of the antigen of interest. One of skill in the art would be knowledgeable as to the parameters that can be modified to increase the signal detected, as well as other variations of ELISAs known in the art. For further discussion regarding ELISAs, see, e.g., Ausubel et al, eds, 1994, Current Protocols in Molecular Biology, Vol. 1, John Wiley & Sons, Inc., New York, at 1 1.2.1.

The binding affinity of an antibody to an antigen and the off-rate of an antibody-antigen interaction can be determined by competitive binding assays. One example of a competitive binding assay is a radioimmunoassay (RIA) involving the incubation of labeled antigen (e.g., ³H or 125I), or a fragment or variant thereof, with the antibody of interest in the presence of increasing amounts of labeled antigen, and the detection of the antibody bound to the labeled antigen. The affinity of the antibody of interest for the NF-κB pathway-associated protein and the binding off rates can be determined from the data by Scatchard plot analysis. Competition with a second antibody can also be determined using RIAs. In this case, the NF-κB pathway-associated protein is incubated with antibody of interest conjugated to a labeled compound (e.g., a compound labeled with ³H or ¹²⁵I) in the presence of increasing amounts of an unlabeled second antibody. This kind of competitive assay between two antibodies, can also be used to determine if two antibodies bind to the same or to different epitopes of the same molecule.

In a preferred aspect, BlAcore kinetic analysis is used to determine the binding on and off rates of antibodies (including antibody fragments or variants thereof) to the NF-κB pathway-associated proteins, or fragments of the NF-κB pathway-associated proteins. Kinetic analysis comprises analyzing the binding and dissociation of antibodies from chips with immobilized NF-κB pathway-associated proteins on the chip surface.

### Methods of Diagnosis of NF-κB Related Disorders and Diseases

The present invention also relates to methods and compositions for the diagnosis of NF-κB pathway-related disorders, diseases and conditions. Such methods comprise, for example, measuring expression of the NF-κB pathway-associated polypeptide genes, or peptide-encoding fragments thereof, in a patient sample, or detecting a mutation in the gene in the genome of an individual suspected of exhibiting NF-κB pathway-related dysfunction. NF-κB pathway-associated nucleic acid molecules can also be used as diagnostic hybridization probes, or as primers, for diagnostic PCR analysis to identify gene mutations, allelic variations, or regulatory defects, such as defects in the expression of the gene, which can serve as indicators of susceptibility to NF-κB pathway disorder, or a lack thereof. Such diagnostic PCR analyses can be used to diagnose individuals with NF-κB disorder associated mutation, allelic variation, or regulatory defects in a NF-κB pathway-associated gene.

Methods of the invention for the diagnosis, screening and/or prognosis of NF-κB pathway-related diseases, disorders and conditions can utilize reagents such as the NF-κB pathway-associated nucleic acid molecules and sequences or antibodies directed against the proteins or polypeptides, including peptide fragments thereof. Specifically, such reagents can be used, for example, for: (1) the detection of the presence of NF-κB pathway-associated polypeptide gene mutations, or the detection of either over- or under-expression of NF-κB pathway-associated polypeptide gene mRNA relative to the disease state, or the qualitative or quantitative detection of alternatively-spliced forms of peptide transcripts which may correlate with NF-κB pathway-related disorders or susceptibility to such disorders; and (2) the detection of either an over- or an under-abundance of the NF-κB pathway-associated gene product relative to the disease state or the presence of a modified (e.g., less than full length) gene product which correlates with a NF-κB pathway dysfunctional state or a progression toward such a state. In addition, such NF-κB pathway-associated reagents can be used in methods for the screening, diagnosis and/or prognosis of diseases, disorders, and/or conditions, that are associated with NF-κB activation, with the activity or function of component molecules of the NF-κB pathway, or with other cell signaling molecules.

The methods described herein may be performed, for example, by utilizing pre-packaged diagnostic test kits comprising at least one specific NF-κB pathway-associated nucleic acid or antibody reagent described herein, which can be conveniently used, e.g., in clinical or laboratory settings, to screen and diagnose patients exhibiting NF-κB pathway-related conditions or symptoms related thereto, or to screen and identify those individuals exhibiting a predisposition or susceptibility to NF-κB pathway related conditions.

For the detection of NF-κB pathway-associated polypeptide mutations, any nucleated cell can be used as a starting source for genomic nucleic acid. For the detection of NF-κB pathway-associated polypeptide transcripts or gene products, any cell type or tissue in which the NF-κB pathway-associated polypeptide genes are expressed can be employed.

### Detection of NF-κB Pathway-Associated Nucleic Acid Molecules

Mutations or polymorphisms within the NF-κB pathway-associated polypeptide genes can be detected by utilizing a number of techniques. As stated above, nucleic acids from any nucleated cell can be used as the starting point for such assay techniques, and can be isolated according to standard nucleic acid preparation procedures which are well known to those of skill in the art.

Genomic DNA can be used in hybridization or amplification assays of biological samples to detect abnormalities involving the NF-κB pathway-associated polypeptide gene structures, including point mutations, insertions, deletions and chromosomal rearrangements. Such assays can include, but are not limited to, direct sequencing (C. Wong et al., 1987, Nature, 330:384-386), single stranded conformational polymorphism analyses (SSCP; M. Orita et al., 1989, Proc. Natl. Acad. Sci. USA, 86:2766-2770), heteroduplex analysis (T. J. Keen et al., 1991, Genomics, 11:199-205; D. J. Perry and R. W. Carrell, 1992), denaturing gradient gel electrophoresis (DGGE; R. M. Myers et al., 1985, Nucl. Acids Res., 13:3131-3145), chemical mismatch cleavage (R. associated G. Cotton et al., 1988, Proc. Natl. Acad. Sci. USA, 85:4397-4401) and oligonucleotide hybridization (R. B. Wallace et al., 1981, Nucl. Acids Res., 9:879-894; R. J. Lipshutz et al., 1995, Biotechniques, 19:442-447).

Diagnostic methods for the detection of NF-κB pathway nucleic acid molecules, in patient samples or other appropriate cell sources, can involve the amplification of specific gene sequences, e.g., by PCR, followed by the analysis of the amplified molecules using techniques well known to those of skill in the art, such as, for example, those listed above. Utilizing analysis techniques such as these, the amplified sequences can be compared to those that would be expected if the nucleic acid being amplified contained only normal copies of the NF-κB pathway-associated genes, in order to determine whether a gene mutation exists, for example, a mutation that correlates with NF-κB pathway-related disorders and conditions or susceptibility for same.

Quantitative and qualitative aspects of NF-κB pathway-associated gene expression can also be assayed. For example, RNA from a cell type or tissue known or suspected to express a NF-κB pathway-associated gene can be isolated and tested utilizing hybridization or PCR techniques as described and known in the art. The isolated cells can be derived from cell culture or from a patient. The analysis of cells taken from culture may be a necessary step in the assessment of cells to be used as part of a cell-based gene therapy technique or, alternatively, to test the effect of compounds on the expression of the gene. Such analyses can reveal both quantitative and qualitative aspects of the expression pattern of the NF-κB pathway-associated genes, including activation or inactivation of gene expression or presence of alternatively spliced transcripts.

In one aspect of such a detection scheme, a cDNA molecule is synthesized from an RNA molecule of interest (e.g., a NF-κB pathway-associated polypeptide, by reverse transcription of the RNA molecule into cDNA). All or part of the resulting cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from the NF-κB pathway-associated nucleic acid sequences. The preferred lengths of such nucleic acid reagents are at least 9-30 nucleotides.

For detection of the amplified product, the nucleic acid amplification can be performed using radioactively or non-radioactively labeled nucleotides. Alternatively, enough amplified product can be made so that the product can be visualized by standard ethidium bromide staining or by utilizing any other suitable nucleic acid staining protocol, or, for example, quantitative PCR. Such RT-PCR techniques can be utilized to detect differences in NF-κB pathway-associated transcript size that may be due to normal or abnormal alternative splicing. In addition, such techniques can be utilized, for example, to detect quantitative differences between levels of full length and/or alternatively-spliced transcripts detected in normal individuals relative to those in individuals exhibiting NF-κB related conditions or disorders, or exhibiting a predisposition to such disorders.

As an alternative to amplification techniques, standard Northern analyses can be performed if a sufficient quantity of the appropriate cells can be obtained. Utilizing such techniques, quantitative as well as size-related differences between NF-κB pathway-associated polypeptide transcripts can also be detected. In addition, it is possible to perform NF-κB pathway-associated gene expression assays in situ, i.e., directly upon tissue sections (fixed and/or frozen) of patient tissue obtained from biopsies or resections, such that no nucleic acid purification is necessary. NF-κB pathway-associated nucleic acid molecules can be used as probes and/or primers for such in situ procedures (see, for example, G. J. Nuovo, 1992, PCR In Situ Hybridization: Protocols And Applications, Raven Press, New York).

### Detection of NF-κB Pathway-Associated Polypeptides, Proteins, or Gene Products

Antibodies directed against wild type or mutant NF-κB pathway-associated gene products, or conserved variants or peptide fragments thereof, as described above, can also be used for the diagnosis and prognosis of NF-κB related disorders. Such diagnostic methods can be used to detect abnormalities in the level of gene expression or abnormalities in the structure and/or temporal, tissue, cellular, or subcellular location of NF-κB pathway-associated polypeptide gene products. Antibodies, or fragments of antibodies, can be used to screen potentially therapeutic compounds in vitro to determine their effects on NF-κB pathway-associated gene expression and peptide production. The compounds that have beneficial effects on NF-κB related disorders can be identified and a therapeutically effective dose determined.

In vitro immunoassays can be used, for example, to assess the efficacy of cell-based gene therapy for the treatment of NF-κB related disorders. For example, antibodies directed against NF-κB pathway-associated polypeptides or peptides may be used in vitro to determine the level of NF-κB pathway-associated gene expression found in cells that have been genetically engineered to produce NF-κB pathway-associated polypeptides or peptides. Such analysis allows for a determination of the number of transformed cells necessary to achieve therapeutic efficacy in vivo, as well as optimization of the gene replacement protocol.

The tissue or cell type to be analyzed generally includes those that are known, or suspected, to express the NF-κB pathway-associated polypeptide genes. Protein isolation methods employed can be those as described in Harlow, E. and Lane, D., 1988, Antibodies: A Laboratory Manual, Cold Spring Harbor Laboratory Press, Cold Spring Harbor, N.Y., for example. The isolated cells can be derived from cell culture or from a patient. The analysis of cells taken from culture may be a necessary step in the assessment of cells to be used as part of a cell-based gene therapy technique or, alternatively, to test the effect of compounds on the expression of the gene.

Preferred diagnostic methods for the detection of the NF-κB pathway-associated gene products or conserved variants or peptide fragments thereof, may involve, for example, immunoassays wherein the gene product or conserved variants, including gene products which are the result of alternatively-spliced transcripts, or peptide fragments, are detected by their interaction with an anti-NF-κB-associated polypeptide -specific antibody. For example, antibodies, or fragments of antibodies, such as described above, can be used to detect both quantitatively or qualitatively the presence of the NF-κB-associated gene product or conserved variants or peptide fragments thereof. The antibodies (or fragments thereof) can also be employed histologically, for example, in immunofluorescence or immunoelectron microscopy, for in situ detection of the NF-κB pathway-associated protein or conserved variants or peptide fragments thereof. In situ detection is carried out by removing a histological specimen from a patient, and applying thereto a labeled antibody according to this invention. The antibody (or antibody fragment) is preferably applied by overlaying the labeled antibody (or fragment) onto a biological sample. Through the use of such a procedure, it is possible to determine not only the presence of the NF-κB pathway-associated gene product, or conserved variants or peptide fragments, but also its distribution in the examined tissue. The skilled practitioner will readily perceive that any of a wide variety of histological methods (such as staining procedures) can be modified in order to achieve such in situ detection.

Immunoassays for detecting NF-κB pathway-associated polypeptides or conserved variants or peptide fragments thereof typically comprise incubating a sample, such as a biological fluid, a tissue extract, freshly harvested cells, or lysates of cells which have been incubated in cell culture, in the presence of a detectably labeled antibody capable of binding NF-κB pathway-associated proteins or conserved variants or peptide fragments thereof, and detecting the bound antibody-protein complex by any of a number of techniques well-known in the art.

The biological sample can be brought into contact with and immobilized onto a solid phase support or carrier such as nitrocellulose, nylon membrane, PVDF membrane, or other solid support that is capable of immobilizing cells, cell particles or soluble proteins. The support can then be washed with suitable buffers followed by treatment with the detectably labeled antibody specific for a NF-κB pathway-associated polypeptide. The solid phase support is washed with the buffer a second time to remove unbound antibody. The amount of bound label on the solid support is then detected by conventional means.

A “solid phase support or carrier” refers to any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

The binding activity of an anti-NF-κB pathway-associated polypeptide antibody can be determined according to well-known methods. Those skilled in the art will be able to determine operative and optimal assay conditions for each determination by employing routine experimentation. One of the ways in which an NF-κB pathway-associated polypeptide -specific antibody can be detectably labeled is by linking the antibody to an enzyme in an enzyme linked immunoassay (ELISA) (A. Voller “The Enzyme Linked Immunosorbent Assay (ELISA)”, 1978, Diagnostic Horizons 2:1-7, Microbiological Associates Quarterly Publication, Walkersville, Md.); A. Voller et al., 1978, J. Clin. Pathol., 31:507-520; J. E. Butler, 1981, Meth. Enzymol., 73:482-523; E. Maggio (ed.), 1980, Enzyme Immunoassay, CRC Press, Boca Raton, Fla.; E. Ishikawa et al., (eds.), 1981, Enzyme Immunoassay, Kgaku Shoin, Tokyo). The enzyme that is bound to the antibody reacts with an appropriate substrate, preferably a chromogenic substrate, in such a manner as to produce a chemical moiety that can be detected, for example, by spectrophotometric, fluorometric or visual means. Examples of enzymes that can be used to detectably label an antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection can also be accomplished by visual comparison of the extent of enzymatic reaction of a substrate compared with similarly prepared standards.

Detection can also be achieved using any of a variety of other immunoassays. For example, by radioactively labeling the antibodies or antibody fragments, it is possible to detect NF-κB pathway-associated proteins or peptides through the use of a radioimmunoassay (RIA) (see, for example, B. Weintraub, Principles of Radioimmunoassays, Seventh Training Course on Radioligand Assay Techniques, The Endocrine Society, March, 1986. The radioactive isotope can be detected by using a gamma counter or a scintillation counter or by autoradiography.

It is also possible to label the antibody with a fluorescent compound. When the fluorescently labeled antibody is exposed to light of the proper wavelength, its presence can then be detected due to fluorescence (emission of light of a different wavelength). Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Similarly, a bioluminescent compound can be used to label antibodies against NF-κB pathway-associated polypeptides. Bioluminescence is a type of chemiluminescence found in biological systems in which a catalytic protein increases the efficiency of the chemiluminescent reaction. The presence of a bioluminescent protein is determined by detecting the presence of luminescence. Illustrative bioluminescent compounds for the purposes of bioluminescent labeling include luciferin, luciferase and aequorin.

### Methods and Compositions for the Treatment of NF-κB-Mediated Diseases and Disorders Linked to NF-κB Pathway-Associated Polypeptides and/or Modulators Thereof

The present invention also relates to methods and compositions for the treatment, amelioration, modulation and/or prevention of NF-κB pathway-related disorders that are mediated or regulated by NF-κB pathway-associated polypeptide expression or function, e.g., polypeptide phosphorylation or activation, interaction with signal transduction molecules or cellular regulatory factor molecules or release, or by NF-κB pathway-associated protein modulation, and the like. Further, NF-κB pathway-associated protein effector functions can be modulated via such methods and compositions. Moreover, as described herein, the present invention relates to the treatment, amelioration, modulation, and/or prevention of a variety of other diseases or disorders involving the modulation of NF-κB activity or function, or the activity or function of NF-κB associated molecules, through NF-κB pathway-associated polypeptides or polypeptide modulation.

The methods in accordance with this aspect of the invention include those that modulate NF-κB pathway-associated polypeptides and polypeptide activity product activity. In certain instances, the treatment will require an increase, enhancement, upregulation or activation of NF-κB pathway-associated polypeptide activity, while in other instances, the treatment will require a decrease, reduction, down-regulation or suppression of NF-κB pathway-associated polypeptide activity. “Increase” and “decrease” refer to the differential levels of NF-κB pathway-associated polypeptide activity relative to polypeptide activity in the cell type of interest in the absence of modulatory treatment. Similarly, an “increase” or “decrease” in NF-κB pathway-mediated activity refers to the differential levels of NF-κB-mediated activity (e.g. transcription, gene expression, signal transduction) relative to NF-κB activity in a cell in the absence of modulatory treatment. Methods that can either increase or decrease NF-κB pathway-associated polypeptide activity and/or NF-κB-mediated events depending on the particular manner in which the method is practiced are further described below.

### Methods Associated with a Decrease of NF-κB Pathway-Associated Protein Activity

Treatment of certain NF-κB pathway-related conditions and disorders can be achieved by methods which serve to decrease NF-κB pathway-associated protein activity. Activity can be decreased directly, e.g., by decreasing the NF-κB pathway-associated gene product, i.e., protein, activity and/or by decreasing the level of gene expression. For example, compounds such as those identified through the methods and assays described above that decrease NF-κB pathway-associated protein activity can be used in accordance with the invention to ameliorate, reduce or abolish symptoms associated with certain NF-κB pathway-related conditions and disorders. As discussed above, such molecules can include, but are not limited to, peptides, including soluble peptides, and small organic or inorganic molecules, i.e., NF-κB pathway-associated protein antagonists. Techniques for the determination of effective doses and administration of such compounds are described herein.

### Antisense, Ribozymes, and Triple Helix Formation

In addition, antisense and ribozyme molecules that inhibit NF-κB pathway-associated gene expression can also be used to reduce the level of NF-κB pathway-associated gene expression, thus effectively reducing the level of protein present in a cell, thereby decreasing the level of protein activity, or modulation that occurs in the cell. In addition, antisense molecules and small interfering RNAs molecules of NF-κB pathway-associated proteins, and the like, can be used to modulate or affect the function of molecules which are regulated or mediated by, interact with, and/or are recipients of downstream effects of NF-κB pathway-associated proteins in a cell. Still further, triple helix molecules can be utilized in reducing the level of NF-κB pathway-associated protein gene expression. Such molecules can be designed to reduce or inhibit either wild type, or if appropriate, mutant NF-κB pathway-associated protein target gene activity. Techniques for the production and use of such molecules are well known to those having skill in the art.

As is understood by the skilled practitioner, antisense approaches involve the design of oligonucleotides (either DNA or RNA) that are complementary to mRNA of the NF-κB pathway-associated protein gene sequence or a portion thereof. In a specific embodiment, the antisense molecules are complementary to the mRNA of the polypeptides encoded by the sequences shown in Tables 1-6. The antisense oligonucleotides will bind to the complementary NF-κB pathway-associated gene mRNA transcripts and prevent translation. Absolute complementarity, although preferred, is not required. A sequence “complementary” to a portion of an RNA, as referred to herein, means a sequence having sufficient complementarity to be able to hybridize with the RNA, and form a stable duplex. In the case of double-stranded antisense nucleic acids, a single strand of the duplex DNA may thus be tested, or triplex formation may be assayed. The ability to hybridize depends upon both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by using of standard procedures and practice to determine the melting point of the hybridized complex.

Oligonucleotides that are complementary to the 5′ end of the message, e.g., the 5′ untranslated sequence up to and including the AUG initiation codon, typically work most efficiently at inhibiting translation. However, sequences complementary to the 3′ untranslated sequences of mRNAs have recently been shown to be effective at inhibiting translation of mRNAs as well. (See, generally, R. Wagner, 1994, Nature, 372:333-335). Thus, oligonucleotides complementary to either the 5′ or 3′ untranslated (UTR), non-coding regions of the NF-κB pathway-associated nucleic acids could be used in an antisense approach to inhibit translation of endogenous NF-κB pathway-associated gene mRNA.

Oligonucleotides complementary to the 5′ untranslated region of the mRNA preferably include the complement of the AUG start codon. Antisense oligonucleotides complementary to mRNA coding regions are less efficient inhibitors of translation, but can be used in accordance with the invention. Whether designed to hybridize to the 5′ UTR, 3′ UTR or coding region of a target or pathway gene mRNA, antisense nucleic acids are preferably at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, at least 26 nucleotides, or at least 50 nucleotides.

Regardless of the choice of target sequence, it is preferred that in vitro studies are first performed to quantify the ability of the antisense oligonucleotide to inhibit gene expression. It is preferred that these studies utilize controls that distinguish between antisense gene inhibition and non-specific biological effects of oligonucleotides. It is also preferred that these studies compare levels of the target RNA or protein with that of an internal control RNA or protein. In addition, results obtained using the antisense oligonucleotide are preferably compared with those obtained using a control oligonucleotide. It is also preferred that the control oligonucleotide is of approximately the same length as the antisense oligonucleotide and that the nucleotide sequence of the control oligonucleotide differs from the antisense sequence no more than is necessary to prevent specific hybridization to the target sequence.

The oligonucleotides can be DNA, RNA, or chimeric mixtures, derivatives, or modified versions thereof, single-stranded or double-stranded. Double stranded RNA's may be designed based upon the teachings of Paddison et al., Proc. Nat. Acad. Sci., 99:1443-1448 (2002); and International Publication Nos. WO 01/29058, and WO 99/32619; which are hereby incorporated herein by reference. The oligonucleotide can be modified at the base moiety, sugar moiety, or phosphate backbone, for example, to improve stability of the molecule, hybridization, etc. The oligonucleotide may also include other appended groups such as peptides (e.g., for targeting host cell receptors in vivo), or agents for facilitating transport across the cell membrane (see, e.g., Letsinger et al., 1989, Proc. Natl. Acad. Sci. USA., 86:6553-6556; Lemaitre et al., 1987, Proc. Natl. Acad. Sci. USA, 84:648-652; PCT Application No. WO 88/09810) or the blood-brain barrier (see, e.g., PCT Application No. WO 89/10134), or hybridization-triggered cleavage agents (see, e.g., Krol et al., 1988, Biotechniques, 6:958-976) or intercalating agents (see, e.g., Zon, 1988, Pharm. Res., 5:539-549). For example, the oligonucleotide can be conjugated to another molecule, e.g., a peptide, hybridization triggered cross-linking agent, transport agent, hybridization-triggered cleavage agent, etc.

Such oligonucleotides can be synthesized by standard methods known in the art, for example, by use of an automated DNA synthesizer (such as are commercially available from Biosearch, Applied Biosystems, etc.). As nonlimiting examples, phosphorothioate oligonucleotides can be synthesized by the method of Stein et al. (1988, Nucl. Acids Res., 16:3209) and methylphosphonate oligonucleotides can be prepared by use of controlled pore glass polymer supports (Sarin et al., 1988, Proc. Natl. Acad. Sci. USA, 85:7448-7451), etc.

The antisense molecules are preferably delivered to cells expressing the NF-κB pathway-associated polypeptide gene in vivo. A number of methods have been developed for delivering antisense DNA or RNA to cells; e.g., antisense molecules can be injected directly into the tissue site, or modified antisense molecules that are designed to target the desired cells (e.g., antisense linked to peptides or antibodies that specifically bind to receptors or antigens expressed on the target cell surface) can be administered systemically. Because it is often difficult to achieve intracellular concentrations of the antisense molecules that are sufficient to suppress translation of endogenous mRNAs, a particular approach utilizes a recombinant DNA construct in which the antisense oligonucleotide is placed under the control of a strong pol III or pol II promoter. The use of such a construct to transfect target cells, ex vivo, in vivo, or in vitro, will result in the transcription of sufficient amounts of single stranded RNAs that will form complementary base pairs with the endogenous NF-κB pathway-associated protein gene transcripts and thereby prevent translation of the NF-κB pathway-associated gene mRNA. For example, a vector can be introduced in vivo such that it is taken up by a cell and directs the transcription of an antisense RNA.

Ribozymes are enzymatic RNA molecules capable of catalyzing the specific cleavage of RNA (For a review, see, e.g., Rossi, J., 1994, Current Biology, 4:469-471). The mechanism of ribozyme action involves sequence-specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins. Ribozyme molecules designed to catalytically cleave NF-κB pathway-associated protein gene mRNA transcripts can also be used to prevent translation of protein gene mRNA and expression of target or pathway genes. (See, e.g., PCT Application No. WO 90/11364; and Sarver et al., 1990, Science, 247:1222-1225).

The ribozymes for use in the present invention also include RNA endoribonucleases (hereinafter referred to as “Cech-type ribozymes”) such as that which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; PCT Patent Application No. WO 88/04300; and Been and Cech, 1986, Cell, 47:207-216). The Cech-type ribozymes have an eight base pair active site that hybridizes to a target RNA sequence, after which cleavage of the target RNA takes place. Encompassed by the present invention are those Cech-type ribozymes that target eight base-pair active site sequences that are present in the NF-κB pathway-associated protein genes.

As in the antisense approach, ribozymes can be composed of modified oligonucleotides (e.g. for improved stability, targeting, etc.) and should be delivered to cells that express the NF-κB pathway-associated protein gene, in vivo, in vitro, or ex vivo. A preferred method of delivery involves using a DNA construct “encoding” the ribozyme under the control of a strong constitutive pol III or pol II promoter, so that transfected cells produce sufficient quantities of the ribozyme to destroy endogenous NF-κB pathway-associated protein gene messages and inhibit NF-κB pathway-associated protein mRNA translation. Because ribozymes, unlike antisense molecules, are catalytic, a lower intracellular concentration is required for efficiency.

Endogenous NF-κB pathway-associated protein gene expression can also be reduced by inactivating or “knocking out” the target and/or pathway gene or its promoter using targeted homologous recombination (see, e.g., Smithies et al., 1985, Nature317:230-234; Thomas & Capecchi, 1987, Cell, 51:503-512; and Thompson et al., 1989 Cell, 5:313-321). For example, a mutant, non-functional NF-κB pathway-associated protein gene (or a completely unrelated DNA sequence) flanked by DNA homologous to the endogenous NF-κB pathway-associated protein gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the NF-κB pathway-associated protein gene. Such techniques can also be utilized to generate NF-κB pathway-related disorders animal models. It should be noted that this approach can be adapted for use in humans provided that the recombinant DNA constructs are preferably directly administered or targeted to the required site in vivo using appropriate viral vectors, e.g., herpes virus vectors.

Alternatively, endogenous NF-κB pathway-associated protein gene expression can be reduced by targeting deoxyribonucleotide sequences complementary to the regulatory region of the gene (i.e., the gene promoter and/or enhancers) to form triple helical structures that prevent transcription of the gene in target cells in the body (see generally, Helene, C., 1991, Anticancer Drug Des., 6(6):569-84; Helene, C., et al., 1992, Ann. N.Y. Acad. Sci., 660:27-36; and Maher, L. J., 1992, Bioassays, 14(12):807-15). Nucleic acid molecules for use in triple helix formation to inhibit transcription should be single stranded and composed of deoxynucleotides. The base composition of these oligonucleotides should be designed to promote triple helix formation via Hoogsteen base pairing rules, which generally require that sizeable stretches of either purines or pyrimidines are present on one strand of the duplex. Nucleotide sequences can be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules can be chosen that are purine-rich, for example, containing a stretch of G residues. These molecules form a triple helix with a DNA duplex that is rich in GC pairs, in which the majority of the purine residues are located on a single strand of the targeted duplex, resulting in GGC triplets across the three strands of the triplex.

Alternatively, the potential sequences that can be targeted for triple helix formation are increased by creating a “switchback” nucleic acid molecule. Switchback molecules are synthesized in an alternating 5′-3′, 3′-5′ manner, such that they base pair with first one strand of a duplex and then with the other, eliminating the necessity for a sizeable stretch of either purines or pyrimidines to be present on one strand of the duplex.

In instances in which the antisense, ribozyme, and/or triple helix molecules described herein are utilized to inhibit mutant NF-κB pathway-associated gene expression, it is possible that the technique may so efficiently reduce or inhibit the transcription (triple helix) and/or translation (antisense, ribozyme) of mRNA produced by normal target gene alleles that the concentration of normal target gene product present may be lower than is necessary for a normal phenotype. In such cases, to ensure that substantially normal levels of NF-κB pathway-associated protein gene activity are maintained, nucleic acid molecules that encode and express NF-κB pathway-associated polypeptides exhibiting normal target gene activity can be introduced into cells via gene therapy methods that do not contain sequences susceptible to whatever antisense, ribozyme, or triple helix treatments are being utilized. In instances in which the target gene encodes an extracellular protein, it may be preferable to co-administer normal target gene protein in order to maintain the requisite level of target gene activity.

Antisense RNA and DNA, ribozyme, and triple helix molecules of the invention can be prepared by any method known in the art, e.g., methods for chemically synthesizing oligodeoxyribonucleotides and oligoribonucleotides that are practiced in the art such as solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters, such as the T7 or SP6 polymerase promoters. Alternatively, antisense cDNA constructs that synthesize antisense RNA constitutively or inducibly, depending on the promoter used, can be introduced into cell lines to form stable cell lines containing the construct.

In addition, well-known modifications to DNA molecules can be introduced into the NF-κB pathway-associated nucleic acid molecules as a means of increasing intracellular stability and half-life. Illustrative modifications include, but are not limited to, the addition of flanking sequences of ribo- or deoxyribo-nucleotides to the 5′ and/or 3′ ends of the molecule, or the use of phosphorothioate or 2′ O-methyl rather than phosphodiesterase linkages within the oligodeoxyribonucleotide backbone.

### Methods for Increasing NF-κB Pathway-Associated Protein Activity

Successful treatment of NF-κB pathway-related conditions and disorders can also be effected, where appropriate, by techniques that result in an increase in the level of NF-κB pathway-associated proteins and/or protein activity. Activity can be increased by, for example, directly increasing NF-κB pathway-associated protein activity and/or by increasing the level of gene expression. For example, modulatory compounds such as those identified through the assays and methods described above that increase NF-κB pathway-associated protein activity can be used, as appropriate, to treat NF-κB pathway-related conditions and disorders. Such molecules can include, but are not limited to peptides, including soluble peptides, and small organic or inorganic molecules, and are typically considered to be NF-κB pathway-associated protein agonists. Such a modulatory compound can be administered to a patient exhibiting NF-κB pathway-related disorders and/or symptoms at a level sufficient to treat the NF-κB pathway-related disorders and symptoms. One of skill in the art will readily know how to determine the concentration of an effective non-toxic dose of the compound using procedures routinely practiced in the art.

Alternatively, in instances in which the compound to be administered is a peptide compound, DNA sequences encoding the peptide compound, i.e., a DNA molecule, can be directly administered to a patient exhibiting a NF-κB pathway-related disorder or symptoms, at a concentration sufficient to produce a level of peptide compound sufficient to ameliorate, reduce or abolish the symptoms of the disorder. Any of the techniques described herein which provide the intracellular administration of compounds, such as, for example, liposome administration, transfection, infection, or direct injection, can be utilized for the administration of such DNA molecules. In the case of peptide compounds which act extracellularly, the DNA molecules encoding such peptides can be taken up and expressed by any cell type, so long as a sufficient circulating concentration of peptide results for the elicitation of a reduction or elimination or amelioration of NF-κB pathway-related conditions or symptoms.

In cases in which the NF-κB pathway-related disorder or condition can be localized to a particular portion or region of the body, the DNA molecules encoding such modulatory peptides can be administered as part of a delivery complex. Such a delivery complex can comprise an appropriate nucleic acid molecule and a targeting means. Such targeting means can comprise, for example, sterols, lipids, viruses or target cell specific binding agents. Viral vectors can include, but are not limited to adenovirus, adeno-associated virus, and retrovirus vectors, in addition to other materials that introduce DNA into cells, such as liposomes. In instances in which NF-κB pathway-related disorder or condition involves an aberrant NF-κB pathway-associated gene or protein, patients can be treated by gene replacement therapy. One or more copies of a normal NF-κB pathway-associated protein gene, or a portion of the gene that directs the production of a normal protein with normal protein function, can be inserted into cells by means of a delivery complex as described above. Such gene replacement techniques can be accomplished either in vivo or in vitro. Techniques which select for expression within the cell type of interest are preferred. For in vivo applications, such techniques can, for example, include appropriate local administration of NF-κB pathway-associated protein gene sequences.

Additional methods that can be used to increase the overall level of NF-κB pathway-associated polypeptide activity, in appropriate conditions in which it is advantageous to do so, include the introduction of appropriate NF-κB pathway-associated protein gene-expressing cells, preferably autologous cells, into a patient at sites and in amounts sufficient to ameliorate, reduce, or eliminate NF-κB pathway-related disorders, conditions, or symptoms. Such cells can be either recombinant or non-recombinant. Among the cell types that can be administered to increase the overall level of NF-κB pathway-associated protein gene expression in an individual are normal cells, which express the NF-κB pathway-associated protein gene. The cells can be administered at the anatomical site of expression, or as part of a tissue graft located at a different site in the body. Such cell-based gene therapy techniques are well known to those skilled in the art (see, e.g., Anderson, et al., U.S. Pat. No. 5,399,349; and Mulligan and Wilson, U.S. Pat. No. 5,460,959).

NF-κB pathway-associated protein gene sequences can also be introduced into autologous cells in vitro. Cells expressing the gene sequences can then be reintroduced, preferably by intravenous administration, into the patient until the disorder is treated and symptoms of the disorder are ameliorated, reduced, or eliminated.

### Additional Modulatory Techniques

The present invention also includes modulatory techniques which, depending on the specific application for which they are utilized, can yield either an increase or a decrease in NF-κB pathway-associated protein activity levels leading to the amelioration, reduction, or elimination of NF-κB pathway-related disorders and conditions, such as those described above.

For example, antibodies exhibiting modulatory capability can be utilized according to the methods of this invention to treat NF-κB pathway-related disorders. Depending on the specific antibody, the modulatory effect can be an increase or decrease in NF-κB pathway-associated protein activity, or in activity of a molecule regulated or modulated by the NF-κB pathway-associated protein, e.g., NF-κB. Specific antibodies can be generated using standard techniques as described above against a full-length wild type or mutant NF-κB pathway-associated polypeptide, or against peptides corresponding to portions of the protein. The antibodies include, but are not limited to, polyclonal, monoclonal, Fab fragments, single chain antibodies, chimeric antibodies, etc.

Lipofectin or liposomes can be used to deliver the antibody or an antibody fragment comprising the Fab region, which binds to epitopic regions of the NF-κB pathway-associated proteins, to cells expressing NF-κB pathway-associated proteins. Where fragments of the antibody are used, the smallest inhibitory fragment which binds to an NF-κB pathway-associated protein binding domain is preferred. For example, peptides having an amino acid sequence corresponding to the domain of the variable region of an antibody that binds to the NF-κB pathway-associated protein can be used. Such peptides can be synthesized chemically, or produced via recombinant DNA technology using methods well known in the art (e.g., see Creighton, 1983, supra and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population using, for example, techniques such as those described in Marasco et al., 1993, Proc. Natl. Acad. Sci. USA, 90:7889-7893.

In another specific embodiment of the present invention, NF-κB-pathway modifiers can be combined with cytotoxic agents for the treatment of diseases including, but not limited to cancer. For example, for the treatment of NFκB diseases, inhibitors of NFκB pathway-associated peptides can be combined with cytotoxic agents such as taxol.

### Pharmaceutical Preparations and Methods of Administration

The compounds, e.g., nucleic acid sequences, proteins, polypeptides, peptides, modulators, and recombinant cells, described above can be administered to a patient, or to an individual in need thereof, in therapeutically effective doses to treat or ameliorate NF-κB pathway-related conditions and disorders. Such compounds are preferably modulators of NF-κB pathway-associated protein, such as antagonists or agonists, more preferably, obtained by methods discussed herein. A therapeutically effective dose refers to that amount of a compound or cell population sufficient to result in amelioration, reduction, elimination, or treatment of the disorder or symptoms. Alternatively, a therapeutically effective amount is that amount of a nucleic acid sequence sufficient to express a concentration of the NF-κB pathway-associated protein product which results in the amelioration of the disorder or symptoms.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, to reduce side effects. The data obtained from the cell culture assays and animal studies can be used in formulating a range of dosage for use in humans.

The dosage of such compounds lies preferably within a range of circulating concentrations that include the ED₅₀ with little or no toxicity. The dosage can vary within this range depending upon the dosage form employed and the route of administration utilized. For any compound used in the methods of the invention, the therapeutically effective dose can be estimated initially from cell culture assays. A dose can be formulated in animal models to achieve a circulating blood or plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Pharmaceutical compositions for use in accordance with the present invention and methods can be formulated in a conventional manner using one or more physiologically acceptable and/or pharmaceutically acceptable carriers, diluents, or excipients. Thus, therapeutic (and preventative) compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Tablets can be coated by methods well known in the art. Liquid preparations for oral administration can take the form of, for example, solutions, syrups or suspensions, or they can be presented as a dry product for reconstitution with water or another suitable vehicle before use. Such liquid formulations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations and formulations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that NF-κB pathway-associated protein-expressing cells be introduced into patients via intravenous administration.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, such as a blister pack. The pack or dispenser device can be accompanied by instructions for administration.

### Animal Models

In accordance with the present invention, NF-κB pathway-associated polynucleotides (e.g. identified in Tables 1-6) can be used to generate genetically altered non-human animals or human cell lines. For example, NF-κB pathway-associated gene products can be expressed in transgenic animals, such as mice, rats, rabbits, guinea pigs, pigs, micro-pigs, sheep, goats, and non-human primates, e.g., baboons, monkeys, and chimpanzees. The term “transgenic” as used herein refers to animals expressing NF-κB pathway-associated nucleic acid sequences from a different species (e.g., mice expressing human NF-κB pathway-associated nucleic acid sequences), as well as animals that have been genetically engineered to over-express endogenous (i.e., same species) NF-κB pathway-associated nucleic acid sequences, or animals that have been genetically engineered to no longer express endogenous NF-κB pathway-associated nucleic acid sequences (i.e., “knock-out” animals), and their progeny.

Transgenic animals can be produced using techniques well known in the art, including, but not limited to, pronuclear microinjection (P. C. Hoppe and T. E. Wagner, 1989, U.S. Pat. No. 4,873,191); retrovirus mediated gene transfer into germ lines (Van der Putten et al., 1985, Proc. Natl. Acad. Sci. USA, 82: 6148-6152); gene targeting in embryonic stem cells (Thompson et al., 1989, Cell, 56: 313-321); electroporation of embryos (Lo, 1983, Mol Cell. Biol., 3: 1803-1814); and sperm-mediated gene transfer (Lavitrano et al., 1989, Cell, 57: 717-723); etc. For a review of such techniques, see Gordon, 1989, Transgenic Animals, Intl. Rev. Cytol., 115: 171-229. In addition, any technique known in the art can be used to produce transgenic animal clones containing a NF-κB pathway-associated protein transgene, for example, nuclear transfer into enucleated oocytes of nuclei from cultured embryonic, fetal or adult cells at quiescence (Campbell et al., 1996, Nature, 380: 64-66; and Wilmut et al., 1997, Nature, 385: 810-813).

Without intending to be in any way limiting, the following further embodiments are encompassed by the present invention:

### Further Embodiments

Embodiment 1: A method of diagnosing, ameliorating, treating, reducing, eliminating, and/or preventing a disease, disorder, and/or condition affected by modulation of NF-κB pathway-associated polypeptide in cells expressing the polypeptide, which comprises providing a modulator of the NF-κB pathway-associated polypeptide in an amount effective to affect the function or activity of the NF-κB pathway-associated polypeptide, and/or to affect the function or activity of NF-κB activation associated with modulated polypeptide activity or function.

The method of embodiment 1, wherein the disease, disorder, and/or condition that can be diagnosed, ameliorated, treated, reduced, eliminated, or prevented includes NF-κB pathway-related disorders and/or conditions, autoimmune disorders, disorders related to hyperimmune activity, inflammatory conditions, COPD, disorders related to aberrant acute phase responses, hypercongenital conditions, birth defects, necrotic lesions, wounds, organ transplant rejection, conditions related to organ transplant rejection, renal diseases, ischemia-reperfusion injury, heart disorders, disorders related to aberrant signal transduction, proliferation disorders, cancers, HIV infection, or HIV propagation in cells infected with other viruses, asthma, cystic fibrosis and pulmonary fibrosis.

The method of embodiment 1, wherein the modulator of NF-κB pathway-associated protein function, activity and/or interaction is an antagonist.

The method of embodiment 1, wherein the modulator of NF-κB pathway-associated protein function, activity and/or interaction is an antagonist selected from drugs, chemical compounds, proteins, peptides, antibodies, ligand compounds, small molecules, antisense complementary nucleic acid molecules, or ribozymes.

The method of embodiment 1, wherein the modulator of NF-κB pathway-associated protein function, activity and/or interaction is an NF-κB pathway-associated protein antagonist which decreases NF-κB activity.

The method of embodiment 1, wherein the modulator of NF-κB pathway-associated protein function, activity and/or interaction is an agonist.

The methods of embodiment 1, wherein the modulator of NF-κB pathway-associated protein function, activity and/or interaction is an agonist selected from drugs, chemical compounds, proteins, peptides, antibodies, ligand compounds, or small molecules.

The method of embodiment 1, wherein the modulator of NF-κB pathway-associated protein function, activity and/or interaction is an NF-κB pathway-associated protein agonist which increases NF-κB activity.

### Additional Embodiments

Embodiment 2: A method of identifying or screening for modulators of NF-κB pathway-associated polypeptides for ameliorating, treating, reducing, eliminating, or preventing NF-κB pathway-related diseases, disorders and/or conditions, comprising testing a compound to determine if the test compound modulates or affects (i) the activity and/or function of the NF-κB pathway-associated protein, (ii) the expression of the protein; and/or (iii) the interaction of the protein with an associated cell molecule in cells exposed to a harmful or deleterious extracellular stimulus.

A method of identifying or screening for modulators of the NF-κB pathway-associated protein for ameliorating, treating, reducing, eliminating, or preventing a disease, disorder and/or condition selected from autoimmune disorders, disorders related to hyperimmune activity, inflammatory conditions, disorders related to aberrant acute phase responses, hypercongenital conditions, birth defects, necrotic lesions, wounds, organ transplant rejection, conditions related to organ transplant rejection, renal diseases, ischemia-reperfusion injury, heart disorders, disorders related to aberrant signal transduction, proliferation disorders, cancers, HIV infection, or HIV propagation in cells infected with other viruses, asthma, cystic fibrosis, or pulmonary fibrosis, comprising testing a compound to determine if the test compound modulates or affects (i) the activity and/or function of the NF-κB pathway-associated polypeptide, (ii) the expression of the polypeptide, and/or (iii) the interaction of the polypeptide with an associated cell molecule in cells in which NF-κB activation is affected.

A method of identifying or screening for modulators of NF-κB pathway-associated polypeptides, wherein modulators comprise compounds and drugs functioning as agonists and antagonists, comprising combining a candidate modulator compound with a host cell expressing the polypeptides encoded by the sequences set forth in Tables 1-6; and measuring an effect of the candidate modulator compound on the activity or function of the expressed NF-κB pathway-associated polypeptide.

A method of screening for or identifying compounds that can modulate the biological activity or function of the NF-κB pathway-associated polypeptide, comprising determining the biological activity of the polypeptide in a cell expressing the polypeptide in the absence of a modulator compound; contacting the host cell expressing the NF-κB pathway-associated polypeptide with the modulator compound; and determining the biological activity or function of NF-κB pathway-associated polypeptide in the presence of the modulator compound; wherein a difference between the activity of the polypeptide in the presence of the modulator compound and in the absence of the modulator compound is indicative of a modulating effect of the compound on NF-κB pathway-associated polypeptide activity or function.

A compound which is a NF-κB pathway-associated polypeptide modulator as identified by the methods of embodiment 2, as well as compositions, including pharmaceutical compositions, comprising the modulator compound.

### Further Embodiments

An isolated polynucleotide encoding a NF-κB pathway-associated polypeptide variant of the polynucleotides set forth in Tables 1-6.

An isolated polynucleotide encoding a NF-κB pathway-associated polypeptide variant of the polypeptides set forth in Tables 1-6.

Compositions, pharmaceutical compositions, vectors and host cells comprising the variant NF-κB pathway-associated amino acid and nucleic acid sequences of these embodiments are encompassed by the invention.

A NF-κB pathway-associated protein peptide derived from the sequences set forth in Tables 1-6.

Antibodies, or fragments thereof, directed against NF-κB pathway-associated polypeptides, peptides, variants, and fragments thereof. The antibodies can be directed against all or a portion of the NF-κB pathway-associated peptides or polypeptides encoded by the sequences shown in Tables 1-6. The antibodies can be of any of the types described herein, including, for example, monoclonal, polyclonal, chimeric, and the like. Methods of utilizing the antibodies in screening assays, in diagnostic assays, as modulators, in detection assays, in purification techniques, and the like, are encompassed.

Compositions and pharmaceutical compositions comprising NF-κB pathway-associated variant polypeptides, peptides and/or antibodies are encompassed by the invention. NF-κB pathway-associated fusion polypeptides and peptides are also encompassed.

### Still Further Embodiments

An isolated nucleic acid molecule that is complementary to all or a portion of the NF-κB pathway-associated nucleic acid sequences set forth in Tables 1-6.

Compositions, pharmaceutical compositions, vectors and host cells comprising the above isolated nucleic acid molecules are encompassed. Probes and primer oligonucleotides as described in the Tables and disclosure herein are also encompassed.

A method of treating a disease, disorder, and/or condition associated with NF-κB activation, or associated with activation of a molecule mediated by NF-κB activation, comprising providing a modulator of a NF-κB pathway-associated protein in a pharmaceutically acceptable formulation, in an amount effective to modulate the expression of NF-κB pathway-associated protein. In the method the modulator is an antagonist or an agonist.

### Additional Embodiments

A method of regulating second messenger pathways and molecules therein, wherein the second messenger pathways and molecules therein are associated with a NF-κB pathway-associated disorder or disease comprising: modulating the function and/or activity of a NF-κB pathway-associated polypeptide. The method comprises regulating, modulating, or affecting the activity of the NF-κB pathway and components thereof by modulating, either by antagonizing or agonizing, the function and/or activity of an NF-κB pathway-associated polypeptide. NF-κB pathway-associated protein modulation according to the method provides treatments for diseases, disorders, and/or conditions that are mediated by NF-κB and/or other molecules related thereto. The method provides treatment, amelioration, or prevention of diseases that are caused by, or are associated with, NF-κB, the NF-κB pathway and/or its component molecules, wherein antagonist modulators of NF-κB pathway-associated proteins are preferably employed to decrease or increase the activity of NF-κB, the NF-κb pathway and/or its component molecules.

### EXAMPLES

The Examples herein are meant to exemplify the various aspects of carrying out the invention and are not intended to limit the scope of the invention in any way.

### Example 1

### Identification of NF-κB Pathway-Associated Polypeptides Utilizing Subtraction Library Technology

### Subtraction Library

### Cell Culture

For the subtraction library, duplicate flasks of THP-1 cells (10⁸) were cultured at 10⁶/ml in RPMI containing 10% heat inactivated fetal calf serum, 2 mM L-glutamine with either medium, or with BMS-205820 (2 uM) for 30 minutes at 37° C. in 5% CO₂. LPS (100 ng/ml) was added to both groups and the cells were cultured for an additional 2 hours. At the end of the incubation, cells were pelleted, washed one time with 10 ml PBS, and stored at −80° C.

### RNA Isolation

Poly A+ mRNA was isolated using the FastTrack 2.0 kit (Invitrogen, Carlsbad, Calif.) according to manufacturer's instructions.

Construction of the Subtraction Library

For first strand synthesis, Oligo d(t) Not (5′-AAGCAGTGGTAACAACGCAGAGTGCGGCCGA(T)₁₅A/G-3′) (SEQ ID NO: 677) and CapSal (5′-AAGCAGTGGTAACAACGCAGAGTCGACrGrGrG-3′) (SEQ ID NO: 678) primers were added to the RNA, and incubated for 2 minutes at 72° C., followed by 2 minutes on ice. The reaction was initiated with dNTPs and SuperScript II (Life Technologies, Baltimore, Md.). The second strand was synthesized using KlenTaq (Clontech, Palo Alto, Calif.), dNTPs, and primer (5′-AAGCAGTGGTAACAACGCAGAGTCGAC-3′ ) (SEQ ID NO: 679). The reaction was purified using a Microspin S-40010 HR column (Amersham Inc., Chicago, Ill.), and double digested with Not I and Sal I. The digested products were size fractionated using a ChromaSpin 100 column (Clontech).

The digested cDNA from the LPS group (tester) was cloned into the vector pSPORT1 precut with Not I and Sal I. The digested cDNA from the LPS plus BMS-205820 group (driver) was cloned into the pSPORT2 vector that was also cut with Not I and Sal I. The tester cDNA library in pSPORT 1 was electroporated into DH12S cells for single strand DNA isolation, and the driver cDNA library was electroporated into DH10B cells. The primary transformants were amplified in semi-solid agar.

Single stranded cDNA from the tester pSPORT1 library was rescued using M13K07 helper phage. DNA was isolated from the amplified driver pSPORT2 library using a Qiagen maxi-prep plasmid kit. The driver library was linearized using Sal I and reverse transcribed with T7 RNA polymerase, rNTPs, and biotin-16-UTP. The biotinylated RNA was treated with RNAse-free DNAse, precipitated, and purified using G-50 spin columns (Bio-Rad, Hercules, Calif.).

Prior to hybridizing the single stranded DNA with the biotinylated RNA, the poly dA region of the single stranded DNA was blocked using a d(T)-Not I oligonucleotide, dTTP nucleotides, and Taq polymerase. The single stranded cDNA was further blocked using Cot-1 DNA (Life Technologies).

For the subtractive hybridization, 600 ng of single stranded tester cDNA (poly dA, Cot-1 blocked pSPORT1) and 80 ug biotinylated driver RNA were used. The biotinylated driver RNA was incubated with hybridization buffer (40% formamide, 50 mM HEPES, 1 mM EDTA, 0.1% SDS) at 65° C. for 10 minutes, followed by 1 minute at 4° C. After this incubation, the tester cDNA was added and the sample was incubated for 24 hours at 42° C. Hybrids were removed by addition of streptavidin followed by phenol/chloroform extractions. The remaining single stranded DNA was precipitated, and used in repair reactions.

The single stranded DNA was repaired using T7 pSPORT primer, dNTPs and Precision-Taq polymerase. The repaired DNA was electroporated into DH12S cells, and then amplified to generate single stranded DNA for a second round of subtraction with the biotinylated driver RNA.

TABLE 1
Sequences that are inhibited by the NF-κB pathway

| Gene | Accession # | Seq ID Nos. |
|---|---|---|
| CLK1 | L29219 | 1 & 2 |
| Cytokine-Inducible Kinase | BC013899 | 3 & 4 |
| GPR85 | AF250237 | 5 & 6 |
| RGS16 | BC006243 | 7 & 8 |
| SDCBP | BC013254 | 9 & 10 |
| BTG1 | NM_001731 | 11 & 12 |
| JTB | NM_006694 | 13 & 14 |
| BCL2L11 | NM_006538 | 15 & 16 |
| BCL-6 | NM_001706 | 17 & 18 |
| EED | U90651 | 19 & 20 |
| similar to lysosomal amino acid | XM_058449 | 21 & 22 |
| transporter 1 | | |
| Truncated Calcium Binding Protein | NM_016175 | 23 & 24 |
| WDR4 | AJ243913 | 25 & 26 |
| FLJ22649 | NM_021928 | 27 & 28 |
| FLJ21313 | NM_023927 | 29 & 30 |
| MGC20791 | XM_046111 | 31 & 32 |
| LOC113402 | NM_145169 | 33 & 34 |
| | (XM_054209) | |
| DKFZp761I241 | AL136565 | 35 & 36 |
| DGCRK6 | AB050770 | 37 & 38 |
| TNF-Induced Protein | BC007014 | 39 & 40 |
| FLJ12120 | AK022182 | 747 & N/A |
| GSA7 | NM_006395 | 749 & 750 |
| HSPC128 | NM_014167 | 751 & 752 |
| C2GNT3 | NM_016591 | 753 & 754 |
| FLJ20512 | NM_017854 | 755 & 756 |
| FLJ11715 | NM_024564 | 757 & 758 |
| LNX | NM_032622 | 759 & 760 |
| FLJ14547 | NM_032804 | 761 & 762 |
| XBP1 | NM_005080 | 763 & 764 |
| IL23A | NM_016584 | 765 & 766 |

TABLE 2
Sequences that are induced by the NF-κB pathway

| Gene | Accession # | Seq ID Nos. |
|---|---|---|
| SGK-like protein SGKL | AF085233 | 41 & 42 |
| KIAA0794 | AB0183370 | 43 & 44 |
| KIAA0456 | AB007925 | 45 & 46 |
| ORPHAN NUCLEAR RECEPTOR TR4 | U10990 | 47 & 48 |
| SUMO-1-specific protease (SUSP1) | NM_015571 | 49 & 50 |
| SUMO-1 activating enzyme subunit 1 | NM_005500 | 51 & 52 |
| | (XM_009036) | |
| BRCA1-associated RING domain protein | U76638 | 53 & 54 |
| (BARD1) | | |
| MGC: 4079 | BC005868 | 55 & 56 |
| FLJ23390 | AK027043 | 57 & 58 |
| MGC19595 | NM_033415 | 767 & 768 |
| GLE1 | NM_001499 | 769 & 770 |
| BLVRA | NM_000712 | 771 & 772 |
| PPP1R7 | NM_002712 | 773 & 774 |
| MADH5 | NM_005903 | 775 & 776 |
| CHS1 | NM_000081 | 777 & 778 |
| ZNF304 | NM_020657 | 779 & 780 |

### Example 2

### NF-κB Pathway-Associated Protein PCR Expression Profiling

### Real Time PCR Analysis

Poly (A)⁺ mRNA was isolated from THP-1 cells that were either unstimulated, or stimulated with 100 ng/ml LPS for two hours in the presence and absence of BMS-205820 (2 uM) using the Fast Track isolation kit (Invitrogen, Carlsbad, Calif.) according to manufacturer's instructions. RNA quality and quantity were evaluated using UV spectrometry and capillary electrophoresis with the RNA 6000 Assay 10 (Agilent). First-strand cDNA was synthesized using the SuperScript™ First-Strand Synthesis System for RT-PCR (Invitrogen) according to manufacturer's instructions.

PCR reactions were performed in a total volume of 40 ul containing master mix (SYBR Green I dye, 50 mM Tris-Cl pH 8.3, 75 mM KCl, DMSO, Rox reference dye, 5 mM MgCl₂, 2 mM dNTP, 1 unit Platinum Taq High Fidelity enzyme), 0.5 uM 15 each of forward and reverse gene-specific primers, and cDNA (8 ul of a 1:36 dilution of the first strand reaction mix). For tissue expression analyses, PCR reactions included 2 ul of cDNA derived from the Human Multiple Tissue cDNA panel I and Human Immune System MTC Panel (Clontech, Palo Alto, Calif.). The amplification program consisted of a 10 minute incubation at 95° C., followed by 40 cycles of incubations at 95° C. for 15 seconds, 60° C. for 1 minute. The amplification was followed by melting curve analysis at 60° C. to determine the specificity of the amplification reaction. The data were analyzed using the TaqMan 5700 software with the threshold value set to 0.5. The message levels of GAPDH were used to normalize the amounts of cDNA for each reaction.

Gene Specific Primers

| | | Seq |
|---|---|---|
| Gene | Specific Primer | ID No. |
| CLK1F | 5′GCTGTGTCCAGATGTTGGAATG3 | 680 |
| CLK1R | 5′CAATGCAAATGTGACCATGATG3′ | 681 |
| Cytokine- | 5′GGCTCTCCTCATGCTGTTTAGTG3′ | 682 |
| Inducible | | |
| Kinase F | | |
| Cytokine- | 5′GTGGGAAGCGAGGTAAGTACAAG3′ | 683 |
| Inducible | | |
| Kinase R | | |
| GPR85F | 5′CGCTCCTTCAGGGCTAATGAT3′ | 684 |
| GPR85R | 5′GCTGTGTGGCTAGGAGGATGAG3′ | 685 |
| RGS16F | 5′GTCCCTTAGCTTGTACCTCGTAACA3′ | 686 |
| RGS16R | 5′TGGCCTTGACATGACTGCAA3′ | 687 |
| SDCBPF | 5′CCCTGCCAATCCAGCAATT3′ | 688 |
| SDCBPR | 5′GCCACACTTGCACGTATTTCTTC3′ | 689 |
| BTG1F | 5′CCAGCAGGAGGTAGCACTCAA3′ | 690 |
| BTG1R | 5′GCTGATTCGGCTGTCTACCATT3′ | 691 |
| JTBF | 5′CGCTCAGCTTTGATGGAACA3′ | 692 |
| JTBR | 5′GTCCAATTGTCGCTGACGAAT3′ | 693 |
| BCL2L11F | 5′GGCGTATCGGAGACGAGTTTAA3′ | 694 |
| BCL2L11R | 5′GGTCTTCGGCTGCTTGGTAA3′ | 695 |
| BCL-6F | 5′GCCATGCCAGTGATGTTCTTC3′ | 696 |
| BCL-6R | 5′CACGGCTCACAACAATGACAA3′ | 697 |
| EEDF | 5′CACTGACAACGTTATGTGTGGTCTT3′ | 698 |
| EEDR | 5′CGAATAGCAGCACCACATTTATGA3′ | 699 |
| Similar to | 5′CTAGGTGTGGTGGTCTGTGCTTAT3′ | 700 |
| lysosomal | | |
| amino acid | | |
| transporter 1F | | |
| Similar to | 5′CCTCCCAACTTATCCTCCAGAGTA3′ | 701 |
| lysosomal | | |
| amino acid | | |
| transporter 1R | | |
| Truncated | 5′GGCCTGACATGGAAGGTGAA3′ | 702 |
| calcium | | |
| binding | | |
| proteinF | | |
| Truncated | 5′CCCATTTAGAGGATGTGGCTGTA3′ | 703 |
| calcium | | |
| binding | | |
| proteinR | | |
| WDR4F | 5′CCGATGACAGTAAGCGTCTGATT3′ | 704 |
| WDR4R | 5′CACGGTCCTGACACTCAGACAT3′ | 705 |
| FLJ22649F | 5′GGTCTGGTGTGCCTTGTCAA3′ | 706 |
| FLJ22649R | 5′CCAGTAGTTCCCAGCCTCCAT3′ | 707 |
| FLJ21313F | 5′CCAGCCAGTACAAGGCCAATAT3′ | 708 |
| FLJ21313R | 5′CCTCCGTTGGGACACTAAGAAAC3′ | 709 |
| MGC20791F | 5′CCATCTCTTGGTTTGGTCACATC3′ | 710 |
| MCG20791R | 5′CGCAGACACTAGCCTAGAACCTATT3′ | 711 |
| LOC113402F | 5′CCATATGCAAGGGATGCAGTTAT3′ | 712 |
| LOC113402R | 5′CGTCAGTTGTTCCTGGAGTGTTT3′ | 713 |
| DKFZp761I241F | 5′GCCTCCTCTGTCTCACCCTTAA3′ | 714 |
| DKFZp761I241R | 5′GGGTGGATGGTATAGGAAGATTCA3′ | 715 |
| DGCRK6F | 5′CAGTGTAGCCCATTCTTGATCCA3′ | 716 |
| DGCRK6R | 5′GCTGCCTTTGACATCCAGAGA3′ | 717 |
| TNF-induced | 5′CCATCAGGTGGATTATACCTTTGAC3′ | 718 |
| proteinF | | |
| TNF-induced | 5′GAATGATTTGGTGCAGCATCTC3′ | 719 |
| proteinR | | |
| SGKLF | 5′CCTTGGATTCTTGGCTTAGAGTAGA3′ | 720 |
| SGKLR | 5′TGGAAGGGATGCTTGTTCTTG3′ | 721 |
| KIAA0794F | 5′CCATCTGTACTCCAGCAAAGTCA3′ | 722 |
| KIAA0794R | 5′ACTGATGAACACGTTGGCAGTT3′ | 723 |
| KIAA0456F | 5′CACACGAGCGATGACGAATG3′ | 724 |
| KIAA0456R | 5′CCCACGTAGTCAAACTTGGCA3′ | 725 |
| TR4F | 5′CTGGTGACCGGATAAAGCAAGT3′ | 726 |
| TR4R | 5′CAGTTCGCCATGCTGTTACAGA3′ | 727 |
| SUSP1F | 5′GAAGATGAACTCGTCGACTTCTCA3′ | 728 |
| SUSP1R | 5′GGAATCCATCGTCACTGCTATCA3′ | 729 |
| SUMO-1 | 5′CCTCCGACTACTTTCTCCTTCAAG3′ | 730 |
| activating | | |
| enzymeF | | |
| SUMO-1 | 5′CCCAGTGAGTCAAGCACATCA3′ | 731 |
| activating | | |
| enzymeR | | |
| BARD1F | 5′GTGAACACCACCGGGTATCAA3′ | 732 |
| BARD1R | 5′GGCTCCATAGGAAAGTAACAGCTT3′ | 733 |
| MGC:4079F | 5′GGAAGGTGGATGAGGCTACATT3′ | 734 |
| MGC:4079R | 5′TGCTTGCTGCTGCTACTGTGT3′ | 735 |
| FLJ23390F | 5′GCTGCATGTCTTCTGAATAGCAA3′ | 736 |
| FLJ23390R | 5′TCCTACGGCATACTGATCCTAGTTT3′ | 737 |
| CHS1F | 5′CCCACGCCGACCTGATTAC3′ | 781 |
| CHS1R | 5′CTAGCCCAAGGCTTGCAATAGT3′ | 782 |
| ZNF304F | 5′GGAAGGTGGATGAGGCTACATT3′ | 783 |
| ZNF304R | 5′TGCTTGCTGCTGCTACTGTGT3′ | 784 |
| MGC19595F | 5′CCACAACCATGCCAAGATGA3′ | 785 |
| MGC19595R | 5′GATGCCAGGGTTATCCAGGAA3′ | 786 |
| Gle1F | 5′GAGAACCAACCTCTGTCTGAGACTT3′ | 787 |
| Gle1R | 5′GAGCTTGCGTCAGGAGATTTG3′ | 788 |
| BLVRAF | 5′CAAGAGGTGGAGGTCGCCTATA3′ | 789 |
| BLVRAR | 5′GGTATTCCACAAGGACGTGCTT3′ | 790 |
| PPP1R7F | 5′CCACGTTCGTCAGGTTCTGA3′ | 791 |
| PPP1R7R | 5′CAGGAGCAACAGGTGGGTTAA3′ | 792 |
| MADH5F | 5′CTGTTCTTTCGGTAGCCACTGA3′ | 793 |
| MADH5R | 5′CCAGCCCAACAATCGCTTTA3′ | 794 |
| FLJ12120F | 5′CAGCCAGGCTTTCAGACATCT3′ | 795 |
| FLJ12120R | 5′GGTCCTTGGCTTAGCGCATAT3′ | 796 |
| GSA7F | 5′CTAGCAGCCCACAGATGGAGTA3′ | 797 |
| GSA7R | 5′GGTCACGGAAGCAAACAACTTC3′ | 798 |
| HSPC128F | 5′GGCTCAAACGTCACTGGAATC3′ | 799 |
| HSPC128R | 5′CAAGCAACGGCTGGTGAACT3′ | 800 |
| C2GNT3F | 5′CCAGCACAATATTTACTGCATCCA3′ | 801 |
| C2GNT3R | 5′TCATGGCAACTTTGAAGGTATCA3′ | 802 |
| FLJ20512F | 5′CATCTCCTTCATGCAGAGTGACAT3′ | 803 |
| FLJ20512R | 5′CCCAGCAGGAAGAAGCCATAT3′ | 804 |
| FLJ11715F | 5′CTACCTTACCCAGCCAGACAAGA3′ | 805 |
| FLJ11715R | 5′GAATGGCATTTCAGGAGTGTACAG3′ | 806 |
| LNXF | 5′CGGTGTGGCATATCGACATGG3′ | 807 |
| LNXR | 5′CGACGAGGTGAACACGTCTTT3′ | 808 |
| FLJ14547F | 5′GTTGCTGGCAGTGTTGTCTCA3′ | 809 |
| FLJ14547R | 5′GCTGTGATCTTCTGTGCCTTCTATC3′ | 810 |
| XBP1F | 5′GCGCTGAGGAGGAAACTGAA3′ | 811 |
| XBP1R | 5′CACTCATTCGAGCCTTCTTTCG3′ | 812 |
| Mouse Stat1F | 5′GTGGGCATCCTTCATGTGAGT3′ | 813 |
| Mouse Stat1R | 5′CCTTGGCAGAAGCTGCAGTAA3′ | 814 |
| Mouse BCL-6F | 5′CGCACAGTGACAAACCATACAA3′ | 815 |
| Mouse BCL-6R | 5′CTGCGCTCCACAAATGTTACA3′ | 816 |
| Mouse | 5′CAAGGCTCAGGAGTCCTGATCT3′ | 817 |
| MGC20791F | | |
| Mouse | 5′GCCAGGATGGTAAATGGTCATC3′ | 818 |
| MGC20791R | | |
| mGAPDHF | 5′CATGGCCTTCCGTGTTCCTA3′ | 819 |
| mGAPDHR | 5′CCTGCTTCACCACCTTCTTGA 3′ | 820 |
| IL-23 alphaF | 5′GACGCGCTGAACAGAGAGAAT3′ | 821 |
| IL-23 alphaR | 5′GCAGCAACAGCAGCATTACAG3′ | 822 |

### Example 3

### Role of Drosophila CLK1 Homolog, DOA, in NF-κB-Dependent Signaling in Drosophila S2 Cells

The Drosophila DOA (CG1658) protein is very similar to CLK1 (Table I, above) across the length of the protein and has significant homology to serine/threonine kinases of the LAMMER class (FIG. 1). Double-stranded RNA-mediated interference (RNAi) directed against DOA mRNA was used to inhibit DOA expression in Drosophila Schneider 2 (S2) cultured cells. The effect of inhibiting DOA expression on an LPS-inducible reporter was tested. LPS activates the NFκB pathway in Drosophila cells, resulting in expression of antimicrobial peptides including attacin.

A stable cell line expressing the attacin promoter linked to luciferase was treated with RNAi specific for either DOA, the Drosophila IKK-2 homolog, the Drosophila p105 homolog Relish, or the Drosophila IkB homolog cactus. The cells 15 were then stimulated with either media or LPS (FIG. 2). RNAi specific for either IKK-2 or Relish significantly inhibited reporter activation, demonstrating that the activity is dependent on NFκB. Consistent with this data, RNAi specific for the NFκB inhibitor cactus significantly upregulated promoter activity. RNAi specific for DOA significantly inhibited reporter activity, suggesting that DOA is involved an NFκB-dependent transcriptional response.

### Methods

Bioinformatic Analysis of the Phylogenetic Position of Drosophila melanogaster Darkener of Apricot (DOA) Relative to Human CDC-Like Kinase (CLK) Genes

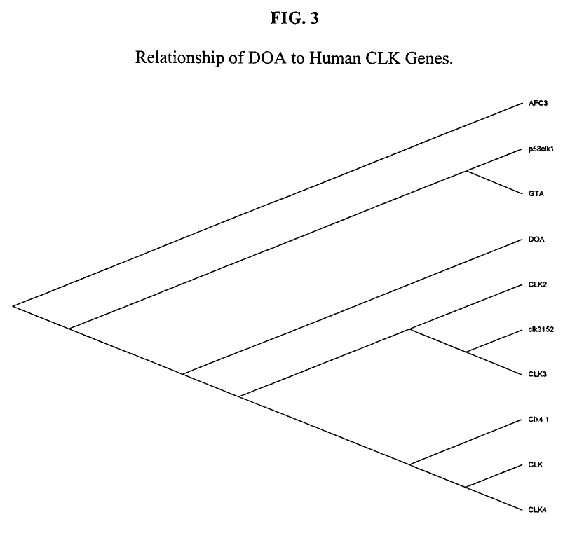

Human protein sequences from CLK1 (gi 632964), CLK2 (gi 632968), CLK3 (gi 632972), CLK4 (9965398), an alternative CLK4 (gi 16157156), a fragment of an alternatively spliced CLK3 (gi 632570), p58clk1 (gi 284345), Drosophila DOA (gi 1706486), Arabidopsis clk gene AFC3 (gi 5915680), and human GalactosylTransferase Associated Protein Kinase (GTA, gi 1170681) were used to construct a phylogenetic tree using the parsimony method. The protein sequences were obtained from the Protein Kinase Resource (http://Ipkr.sdsc.edu). The data were first multiply aligned by Clustal W. PAUP* 4,0b10 for the Macintosh (Sinaur Associates, Sunderland, Mass.) was used to perform the phylogenetic analysis itself. FIG. 3 shows the relationship of DOA to human CLK genes.

### RNAi Analysis in S2 Stable Cell Line Expressing the Attacin Promoter-Lucificerace Construct.

A stable S2 cell line was generated with an LPS-responsive AttacinD promoter fused to a luciferase reporter. S2 cells were purchased from InVitrogen and maintained at 25° C. in complete 1×Schneider's Drosophila medium ( Cat. No. 11720-034, Invitrogen, former GIBCO BRL) supplemented with 10% heat-inactivated fetal bovine serum (Cat. No. 10100-147, Invitrogen, former GIBCO BRL), 100 units/ml of penicillin, 100 ug/ml of streptomycin (100×stock of Penicillin-Streptomycin, cat. No. 15140-148, from Invitrogen, former GIBCO BRL) and 20 mM L-Glutamine (100×L-Glutamine, cat.No. 25030-149, from Invitrogen, former GIBCO BRL). A 1.6 Kb promoter region of the attacinD AMP gene was isolated from S2 genomic DNA by PCR using the primer pair: ATGAGGCTTGGATCAGCTTT (SEQ ID NO: 738) (forward, 157904-157923 bp of AE003718 Drosophila Genome project) and CCTGAAGCCTGACATTCCAT (SEQ ID NO: 739) (reversed, 159547-159566bp of AE003718). Primers were obtained from GIBCOBRL. PCR conditions: 96° C. 4min, 94° C. 2 min, 55° C. 45 seconds, 72° C. 2 min, PCR 35 cycles. The 1.6kb attacinD PCR fragment was subcloned into a pCR2.1-TOPO vector (TOPO TA Cloning Kits, cat. No. K4500-01, Invitrogen). The attacinD promoter was subcloned from pCR2.1-TOPO vector into pGL3-Enhancer luciferase vector with restriction enzyme Sac I and Xho I(pGL3-Enhancer luciferase reporter vector, cat.no. E1771, Promega). A similar region was shown to be LPS responsive in a reporter assay (Tauszig et al., 2000). A final transfection construct, pGL3-enhancer-attacinD, was cotransfected with calcium phosphate methods with pCoHYGRO plasmid providing the hygromycin-B resistant gene as a stable selection, were used to transfect S2 cells (Inducible DES Kit, cat. No. K4120-01, Drosophila Expression System Instruction Manual,p16 from Invitrogen).

Briefly 19 ug of pGL3-enhancer-attacinD DNA was mixed with 1 ug of pCoHYGRO DNA and transfection buffer were used to transfect 6-12×10⁶ cells/3 mls/well in 6-well Falcon tissue culture plate. Stable cells were selected and maintained in complete Schneider's medium containing 300 ug/ml Hygromycin-B (Cat. R220-05, Invitrogen). Stable lines were tested for responsiveness to LPS (Han and Ip, 1999). Cells were treated with 20 ug/ml LPS ( Cat. No L-2654, Sigma) for 5 hours. Expression of luciferase was assayed with Bright-Glo™ Luciferase Assay System ( cat. No. E2620, Promega) and the luminescence signal was detected by 1450 MICROBETA Wallac Jet Liquid Scintillation & Luminescence Counter (Perkin Elmer Life Sciences).Two stable AttD-luc reporter cell lines (E4-1 and E4-9) were obtained after three rounds of limiting dilution and used for further studies.

RNAi constructs were made for DOA and control genes as follows. Complementary DNA (cDNA) clones for Drosophila genes were obtained from Research Genetics, Inc (St. Louis, Mo.). These include the cDNAs from Relish (EST GH01881), IKKB (EST LD21354), Cactus (LD18620), and DOA (LD31161) (Rubin et al., 2000). Double-stranded RNAi generation followed a modified protocol of (Hammond et al., 2000). Briefly, dsRNA was synthesized from a template amplified by PCR with T7 promoter sequences flanking the cDNA insert using the MEGAscript™ T7 High Yield Transcription Kit (cat. No. 1334, Ambion). GH01881, LD 21354, and LD31161 were in the pOT2 vector (forward primer: ACTGCAGCCGATTCATTAATG (SEQ ID NO: 740), reverse primer: GAATTAATACGACTCACTATAGGGAGATATCATACACATACGATTTAG (SEQ ID NO: 741) and LD18620 was in a pBS vector (forward primer: GAATTAATACGACTCACTATAGGGAGACATGATTACGCCAAGCTCGAA (SEQ ID NO: 742) reverse primer: TGTAAAACGACGGCCAGTGAA (SEQ ID NO: 743). dsRNA was diluted at 1:5 and denatured prior to addition to E4-1 and E4-9 cells.

Transfection of dsRNA into S2 cells was performed by adding dsRNA directly into S2 cells in serum free medium (Clemens et al., 2000). Prior to transfection, cells were split about 24 hours before transfection at 1×10⁶ cells/ml in complete 1× Schneider medium. Immediately preceding the transfection, cells were washed twice with serum free DES Expression Medium (cat. No. Q500-01, Invitrogen) and resuspended in serum free DES medium at 7×10⁵ cells/ml. 100 ul cells were added to each well in 96-well-plates (Falcon tissue culture plates), then 5 ul dsRNA/well was added, followed by vigorous shaking for 45 minutes to 1 hour, and then 150 ul complete 1×Schneider medium/well was added. 96-well-plates were wrapped with Saran wrap before incubating at 25° C. After 3 days incubation, each dsRNA treated cells were split into duplicate for the luciferase assay, and in triplicate for the proliferation assay.

5-15 ul cells in 100 ul total volume for were used for the luciferase assay, and 30-35 ul cells in 100 ul total volume were used for the proliferation assay. Luciferase assay plates were incubated for 5 hours after adding LPS at 20 ug/ml. Proliferation assay plates were incubated for 2-3 hours before reading 490 nm Optical Density. (CellTiter 96 Aqueous One Solution Cell Proliferation Assay from Promega, Cat. No. G3580).

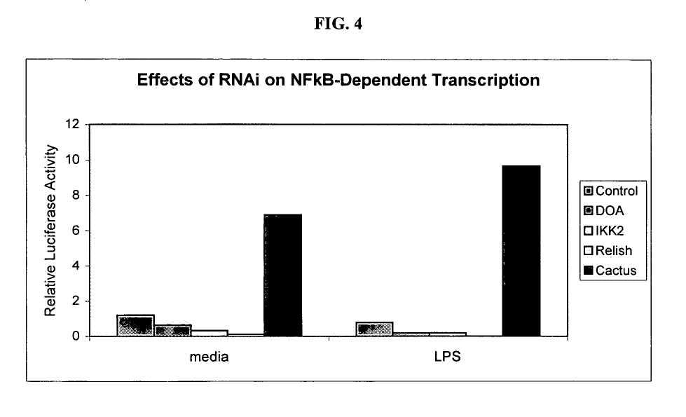

FIG. 4 shows the effects of RNAi on NFκB-dependent Transcription. Results represent one experiment with E4-1 cells averaged in duplicate relative to control samples. The relative luciferase activity is normalized to cell number data obtained in the proliferation assay. Similar results were obtained in 4 separate experiments and with the E4-9 stable cell line. NS is nonstimulated, LPS represents LPS treatment as described above.

### Example 4

### Identification of Addtional NF-κB Pathway-Associated Protein Sequences Following Treatment of Cells with A NF-κB Pathway Inhibitor Utilizing Microarray Technology

### Methods

### Cell culture

THP-1 cells (5×10⁶) were cultured in triplicate at 10⁶/ml in RPMI containing 10% heat inactivated fetal calf serurn, 2 mM L-glutamine with either medium, or with BMS-205820 (2 uM) for 2 hours at 37° C. in 5% CO₂. LPS (100 ng/ml) was added to both groups and the cells were cultured for an additional 2 and 8 hours. One group of triplicates was cultured for 2 and 8 hours with medium alone. At the end of the incubation, cells were pelleted, washed one time with 10 ml PBS, and stored at −80° C. The cell pellets were lysed, and RNA was isolated using the Qiagen RNeasy kit according to manufacturer's instructions.

### Probe Preparation

The RNA was treated in a total reaction volume of 100 ul with RNase Inhibitor (Invitrogen Corp., Carlsbad, Calif.), DNase I (Ambion, Houston, Tex.) for 30 minutes at 37° C. The treated RNA was purified using Qiagen RNease mini columns according to the manufacturer's instructions. For the first strand cDNA synthesis, the RNA was incubated with T7-(dT)24 primer: (5′GGCCAGTGAATTGTAATACGACTCACTATAGGGAGGCGGTTTTTTTTTT TTTTTTTTTTTTTT3′) (SEQ ID NO: 744) for 10 minutes at 70° C., followed by one minute on ice. First strand buffer, DTT, dNTP and RNase were added, and the samples incubated for 2 minutes at 45° C. Superscript II reverse transcriptase (Invitrogen Corp, Carlsbad, Calif.) was added, and the samples incubated for an additional 60 minutes at 45° C.

For the second strand synthesis, the first strand cDNA was incubated with second strand buffer, dNTPs, E. coli ligase, E. coli RNase H, E. coli Polymerase I in a total volume of 150 ul for two hours at 16° C. T4 polymerase was added, and the incubation continued for an additional 5 minutes. Following this incubation, EDTA was added, and the samples placed on ice. The cDNA samples were extracted with phenol:chloroform:isoamyl alcohol and precipitated by addition of 0.5 volumes of 7.5 M ammonium acetate and 2.5 volumes of 100% ethanol. The samples were pelleted by a 30 minute room temperature spin at 12,000×g. The pelleted samples were washed with 0.5 ml 80% ethanol, spun for 10 minutes at 12,000×g, and air dried. The samples were resuspended in 12 ul RNase free water.

The cDNA was labeled using the Enzo Bio Array High Yield RNA transcript labeling kit (Enzo Therapeutics, Farmingdale, N.Y.). The cDNA was incubated with HY reaction buffer, biotin labeled NTP, DTT, RNase mix, and T7 DNA polymerase for six hours at 37° C. Unincorporated nucleotides were removed using Qiagen RNeasy columns according to manufacturer's instructions. The cRNA was fragmented by addition of fragmentation buffer, and incubated for 35 minutes at 95° C. The fragmented cRNA (0.05 mg/ml) was added to a hybridization solution master mix that included 0.1 mg/ml herring sperm DNA, 5 nM oligo B2, 1× standard curve pool, 0.5 mg/ml acetylated BSA, 1×MES hybridization buffer.

The Affymetrix human hg-U133a and hg-U133b chips were probed with the hybridization master mix. The hybridization, washing, and Phycoerythrin streptavidin staining were performed using the Affymetrix hybridization oven and fluidics workstation according to manufacturer's instructions. Stained chips were scanned on the Affymetrix GeneChip scanner, and data was analyzed using the Affymetrix GeneChip software to determine the specifically hybridizing signal for each gene. The differentially expressed genes demonstrated at least a two-fold change in signal when comparing between samples. The differences were all statistically significant (p<0.01) when compared to controls using a T-test.

TABLE 3
Genes whose expression is repressed after 2 hours in the presence
of the NFκB inhibitor BMS-205820

| | | | | | LPS/BMS- |
|---|---|---|---|---|---|
| Gene | Accession # | Seq ID Nos. | Unstimulated | LPS | 205820 |
| CTGF | NM_001901 | 59 & 60 | 49/27 | 3104/483 | 270/78 |
| EGR3 | NM_004430 | 61 & 62 | 178/27 | 1743/66 | 456/80 |
| MINOR | U12767 | 63 & 64 | 28/13 | 2736/273 | 529/170 |
| bcl-6 | U00115 | 65 & 66 | 48/16 | 502/98 | 244/142 |
| NFIL3 | NM_005384 | 67 & 68 | 345/36 | 1313/381 | 588/116 |
| STAT4 | NM_003151 | 69 & 70 | 256/16 | 523/67 | 194/53 |
| NFE2L2 | NM_006164 | 71 & 72 | 799/299 | 4850/718 | 2624/384 |
| KLF5 | NM_001730 | 73 & 74 | 283/104 | 1333/187 | 742/107 |
| EGR2 | NM_000399 | 75 & 76 | 63/40 | 685/35 | 168/138 |
| IGFBP1 | M31159 | 77 & 78 | 184/73 | 6360/195 | 3205/330 |
| IGFBP3 | NM_000598 | 79 & 80 | 50/33 | 2698/398 | 1257/290 |
| LOC57826 | NM_021183 | 81 & 82 | 203/30 | 607/38 | 272/19 |
| PSTPIP2 | NM_024430 | 83 & 84 | 229/83 | 642/23 | 394/94 |
| GEM | NM_005261 | 85 & 86 | 104/23 | 1072/145 | 417/160 |
| PROL2 | NM_006813 | 87 & 88 | 268/124 | 1325/274 | 808/73 |
| PPP1R15A | NM_014330 | 89 & 90 | 381/129 | 3657/37 | 2090/107 |
| PTGER2 | NM_000956 | 91 & 92 | 56/49 | 458/100 | 210/106 |
| transprenyltransferase | NM_014317 | 93 & 94 | 444/85 | 2284/180 | 811/240 |
| DUSP2 | NM_004418 | 95 & 96 | 121/128 | 1323/169 | 484/22 |
| FACL2 | NM_021122 | 97 & 98 | 268/56 | 1118/66 | 457/45 |
| lipoprotein lipase | NM_000237 | 99 & 100 | 844/257 | 5045/1293 | 2378/372 |
| Usurpin-beta | AF015451 | 101 & 102 | 490/234 | 1388/123 | 739/59 |
| BTG2 | NM_006763 | 103 & 104 | 892/72 | 18481/872 | 4784/744 |
| KCNJ2 | AF153820 | 105 & 106 | 200/28 | 3534/327 | 1048/139 |
| SLC7A1 | NM_003045 | 107 & 108 | 36/44 | 124/76 | 28/10 |
| SLC2A6 | NM_017585 | 109 & 110 | 246/99 | 4286/382 | 2796/651 |
| ATP2C1 | AF225981 | 111 & 112 | 76/47 | 272/59 | 109/48 |
| ninjurin 1 | NM_004148 | 113 & 114 | 572/111 | 4156/52 | 2897/157 |
| TPD52 | NM_005079 | 115 & 116 | 186/92 | 451/60 | 198/32 |
| TNFAIP6 | NM_007115 | 117 & 118 | 42/56 | 9298/570 | 2066/45 |
| DSCR1 | NM_004414 | 119 & 120 | 237/78 | 10443/1384 | 1321/138 |
| mader | AJ011081 | 121 & 122 | 82/23 | 467/62 | 211/51 |
| hSIAH2 | U76248 | 123 & 124 | 396/172 | 1252/216 | 373/92 |
| NAV3 | NM_014903 | 125 & 126 | 160/172 | 1150/160 | 175/156 |
| FLJ23231 | NM_025079 | 127 & 128 | 207/170 | 3470/273 | 1756/72 |
| phafin 2 | NM_024613 | 129 & 130 | 274/107 | 815/71 | 429/158 |
| KIAA0346 | AB002344 | 131 & 132 | 112/34 | 1099/38 | 627/114 |
| ADAMTS1 | AF170084 | 133 & 134 | 93/32 | 196/50 | 88/65 |
| MGC: 23129 | BC015663 | 135 & 136 | 288/30 | 3955/231 | 1337/94 |
| DKFZp434M0126 | AL137384 | 137 & 138 | 58/39 | 116/14 | 19/5 |
| FLJ23342 | NM_024631 | 139 & 140 | 68/11 | 126/23 | 60/38 |
| RINZF | NM_023929 | 141 & 142 | 63/52 | 302/35 | 149/16 |
| MGC: 26709 | BC024009 | 143 & 144 | 36/5 | 1193/301 | 49/7 |

Values represent averages and standard deviations of normalized expression values for three replicates per group.

TABLE 4
Genes whose expression is repressed after 8 hours in the presence
of the NFκB inhibitor BMS-205820

| | | | | | LPS/BMS- |
|---|---|---|---|---|---|
| Gene | Accession # | Seq ID Nos. | Unstimulated | LPS | 205820 |
| CXCL13 | NM_006419 | 145 & 146 | 13/11 | 218/64 | 60/52 |
| adrenomedullin | NM_001124 | 147 & 148 | 132/58 | 440/40 | 204/8 |
| FGF4 | M17446 | 149 & 150 | 234/176 | 567/97 | 252/184 |
| I-TAC | AF030514 | 151 & 152 | 45/42 | 159/58 | 23/29 |
| SLC1A3 | NM_004172 | 153 & 154 | 312/50 | 819/157 | 480/28 |
| sorting nexin 11 | NM_013323 | 155 & 156 | 487/71 | 1718/295 | 659/70 |
| BLAME | NM_020125 | 157 & 158 | 122/80 | 1526/128 | 926/70 |
| SLC2A6 | NM_017585 | 109 & 110 | 266/57 | 4545/373 | 1386/77 |
| LY6E | NM_002346 | 161 & 162 | 212/99 | 1614/121 | 580/24 |
| WSX1 | NM_004843 | 163 & 164 | 524/93 | 1211/158 | 802/187 |
| LAMP3 | NM_014398 | 165 & 166 | 30/23 | 2803/667 | 241/99 |
| sialoadhesin | NM_023068 | 167 & 168 | 21/9 | 746/85 | 210/176 |
| PTGER4 | NM_000958 | 169 & 170 | 708/74 | 1663/168 | 833/152 |
| IL18RAP | NM_003853 | 171 & 172 | 117/79 | 617/101 | 160/146 |
| TNFRSF9 | NM_001561 | 173 & 174 | 161/60 | 967/230 | 439/33 |
| SLC5A3 | NM_006933 | 175 & 176 | 625/174 | 1526/113 | 1049/147 |
| ATP1B1 | NM_001677 | 177 & 178 | 282/66 | 1303/121 | 846/66 |
| IGSF6 | NM_005849 | 179 & 180 | 224/43 | 382/67 | 118/38 |
| MDR/TAP | NM_000593 | 181 & 182 | 633/1 | 4606/206 | 1649/308 |
| BIGMo-103 | AB040120 | 183 & 184 | 739/43 | 2505/278 | 1257/86 |
| GRM6 | NM_000843 | 185 & 186 | 276/76 | 508/154 | 270/58 |
| NRCAM | NM_005010 | 187 & 188 | 99/17 | 245/37 | 127/45 |
| SLC11A2 | NM_000617 | 159 & 160 | 76/37 | 444/94 | 126/65 |
| SLC6A8 | NM_005629 | 189 & 190 | 960/198 | 1566/400 | 572/138 |
| STEAP | NM_012449 | 191 & 192 | 108/37 | 255/52 | 66/60 |
| EPCR | NM_006404 | 193 & 194 | 118/19 | 212/21 | 85/36 |
| LILRB1 | NM_006669 | 195 & 196 | 235/133 | 753/146 | 458/175 |
| ninjurin 1 | NM_004148 | 113 & 114 | 391/75 | 2439/287 | 1673/303 |
| PDGFRL | NM_006207 | 197 & 198 | 52/39 | 244/89 | 134/62 |
| NET-6 | NM_014399 | 199 & 200 | 94/55 | 319/60 | 186/115 |
| IL10RA | NM_001558 | 201 & 202 | 411/31 | 3200/543 | 2109/13 |
| PHT2 | NM_016582 | 203 & 204 | 72/98 | 1040/278 | 632/32 |
| GPR51 | NM_005458 | 205 & 206 | 136/38 | 201/33 | 42/63 |
| FSCN1 | NM_003088 | 207 & 208 | 1229/97 | 3379/230 | 1859/50 |
| TNFSF10 | NM_003810 | 209 & 210 | 288/51 | 780/105 | 259/64 |
| BNIP3 | NM_004052 | 211 & 212 | 365/24 | 599/181 | 204/40 |
| optineurin | NM_021980 | 213 & 214 | 234/15 | 1292/23 | 444/117 |
| MGC: 12451 | BC005352 | 215 & 216 | 666/108 | 1514/87 | 555/101 |
| TNFAIP6 | NM_007115 | 117 & 118 | 70/62 | 2420/125 | 143/86 |
| TNF-induced protein | NM_014350 | 217 & 218 | 442/38 | 1364/195 | 606/128 |
| (GG2-1) | | | | | |
| IFI44 | NM_006417 | 219 & 220 | 151/27 | 2367/132 | 546/28 |
| SP110 | NM_004509 | 221 & 222 | 931/36 | 3729/110 | 1408/111 |
| MX1 | NM_002462 | 223 & 224 | 232/111 | 10689/701 | 2330/268 |
| IFI16 | NM_005531 | 225 & 226 | 259/129 | 1348/175 | 444/42 |
| IFI16b | AF208043 | 227 & 228 | 246/68 | 2376/444 | 572/11 |
| IFITM1 | NM_003641 | 229 & 230 | 423/172 | 4335/265 | 1199/108 |
| ISG15 | NM_005101 | 231 & 232 | 621/59 | 11296/154 | 3739/131 |
| RIG-I | NM_014314 | 233 & 234 | 49/31 | 1140/115 | 287/95 |
| interferon-induced | BC001356 | 235 & 236 | 816/104 | 3106/158 | 1441/269 |
| protein 35 | | | | | |
| OAS2p71 | NM_016817 | 237 & 238 | 319/128 | 3744/364 | 1205/283 |
| OAS3 | NM_006187 | 239 & 240 | 463/147 | 5780/88 | 1832/353 |
| PLSCR1 | NM_021105 | 241 & 242 | 554/38 | 3009/173 | 1284/84 |
| OAS2p69 | NM_002535 | 243 & 244 | 154/28 | 828/41 | 312/28 |
| OAS1 | NM_016816 | 245 & 246 | 69/14 | 2033/277 | 650/92 |
| OASL | NM_003733 | 247 & 248 | 217/138 | 1359/76 | 588/100 |
| tryptophanyl-tRNA | NM_004184 | 249 & 250 | 1293/88 | 4998/382 | 2565/86 |
| synthetase | | | | | |
| MX2 | NM_002463 | 251 & 252 | 580/74 | 3964/286 | 1850/227 |
| IFI27 | NM_005532 | 253 & 254 | 88/72 | 562/117 | 168/111 |
| IFIT4 | NM_001549 | 255 & 256 | 37/17 | 1174/225 | 495/53 |
| G1P3 | NM_022873 | 257 & 258 | 202/61 | 2696/429 | 1374/115 |
| TRIM34 | NM_021616 | 259 & 260 | 73/68 | 445/128 | 130/124 |
| HCK | NM_002110 | 261 & 262 | 1390/246 | 6368/634 | 3410/448 |
| UGCG | NM_003358 | 263 & 264 | 431/104 | 1894/26 | 861/41 |
| carboxypeptidase M | NM_001874 | 265 & 266 | 583/27 | 2355/436 | 774/26 |
| ALOX5AP | NM_001629 | 267 & 268 | 1901/113 | 4966/802 | 3036/324 |
| FACL2 | NM_021122 | 97 & 98 | 402/52 | 1192/186 | 527/111 |
| LYN | NM_002350 | 269 & 270 | 597/42 | 2216/253 | 1371/91 |
| CKB | NM_001823 | 271 & 272 | 510/101 | 2149/61 | 1339/101 |
| PRKR | NM_002759 | 273 & 274 | 293/39 | 1608/353 | 812/292 |
| USP18 | NM_017414 | 275 & 276 | 60/20 | 814/48 | 193/76 |
| PLA2G7 | NM_005084 | 277 & 278 | 129/40 | 413/52 | 183/76 |
| LAP3 | NM_015907 | 279 & 280 | 1268/44 | 4220/77 | 2155/134 |
| kynurenine 3- | BC005297 | 281 & 282 | 247/83 | 623/110 | 357/85 |
| hydroxylase | | | | | |
| CHST2 | NM_004267 | 283 & 284 | 125/90 | 706/90 | 333/33 |
| CYBB | NM_000397 | 285 & 286 | 158/106 | 987/49 | 504/33 |
| QPCT | NM_012413 | 287 & 288 | 70/58 | 272/9 | 120/5 |
| PDE4B | L20966 | 289 & 290 | 102/62 | 388/119 | 143/95 |
| UBE2L6 | NM_004223 | 291 & 292 | 455/94 | 2162/276 | 1116/119 |
| IL1BCE | U13699 | 293 & 294 | 275/209 | 898/86 | 354/85 |
| ADAMDEC1 | NM_014479 | 295 & 296 | 101/65 | 502/41 | 251/27 |
| PPM1B2 | AJ271832 | 297 & 298 | 50/43 | 172/35 | 77/30 |
| C1GALT1 | NM_020156 | 299 & 300 | 117/66 | 270/95 | 121/28 |
| ME1 | NM_002395 | 301 & 302 | 267/86 | 1896/370 | 1297/195 |
| PDE4D2 | AF012074 | 303 & 304 | 56/44 | 150/50 | 87/14 |
| spermidine/spermine | M55580 | 305 & 306 | 459/69 | 2948/177 | 1713/333 |
| N1-acetyltransferase | | | | | |
| ARSB | NM_000046 | 307 & 308 | 121/49 | 405/109 | 271/51 |
| sphingosine-1- | AF144638 | 309 & 310 | 145/125 | 404/88 | 240/85 |
| phosphate lyase | | | | | |
| ADAM28 | NM_021777 | 311 & 312 | 44/34 | 131/44 | 75/17 |
| acyl-coenzyme A: | L21934 | 313 & 314 | 69/63 | 219/64 | 96/74 |
| cholesterol | | | | | |
| acyltransferase | | | | | |
| SPPH1 | NM_030791 | 315 & 316 | 44/39 | 142/35 | 78/48 |
| TNIP1 | NM_006058 | 317 & 318 | 703/40 | 5250/152 | 1739/268 |
| SAMSN1 | NM_02213 | 319 & 320 | 98/85 | 557/36 | 185/74 |
| | (NM_02213) | | | | |
| pleckstrin | NM_002664 | 321 & 322 | 1810/134 | 6332/1336 | 3571/197 |
| cyclin E2 splice | AF112857 | 323 & 324 | 227/22 | 503/124 | 240/59 |
| variant 1 | | | | | |
| IFNGR2 | NM_005534 | 325 & 326 | 2395/127 | 9461/365 | 6318/957 |
| PSTPIP2 | NM_024430 | 83 & 84 | 296/120 | 1029/182 | 411/76 |
| calgranulin A | NM_002964 | 327 & 328 | 6170/863 | 19314/2121 | 3722/444 |
| calgranulin B | NM_002965 | 329 & 330 | 2483/215 | 9522/742 | 2000/100 |
| cyclin-E binding | NM_016323 | 331 & 332 | 191/81 | 745/127 | 306/25 |
| protein 1 | (NM_01632) | | | | |
| calgranulin C | NM_005621 | 333 & 334 | 275/59 | 1028/83 | 377/146 |
| HPAST | AF001434 | 335 & 336 | 402/146 | 2909/451 | 773/54 |
| RGS13 | AF030107 | 337 & 338 | 19/23 | 104/29 | 39/29 |
| SCHIP1 | NM_014575 | 339 & 340 | 48/34 | 136/58 | 44/15 |
| CKIP-1 | NM_016274 | 341 & 342 | 289/153 | 2075/310 | 1221/175 |
| ARHE | NM_005168 | 343 & 344 | 94/90 | 834/135 | 546/38 |
| BRDG1 | NM_012108 | 345 & 346 | 59/40 | 212/28 | 58/43 |
| RGL | AF186779 | 347 & 348 | 139/85 | 1675/194 | 1141/165 |
| Sp110b | AF280094 | 349 & 350 | 731/138 | 4588/393 | 1369/47 |
| ISGF-3 | M97935 | 351 & 352 | 686/24 | 3257/176 | 987/56 |
| CREBL2 | NM_001310 | 353 & 354 | 191/59 | 525/65 | 196/47 |
| IRF7 | NM_004030 | 355 & 356 | 378/30 | 3295/413 | 1366/202 |
| NFE2L2 | NM_006164 | 71 & 72 | 826/167 | 3105/304 | 1389/236 |
| MTF1 | NM_005955 | 357 & 358 | 678/82 | 2407/174 | 1173/204 |
| TFEC | NM_012252 | 359 & 360 | 657/67 | 1596/305 | 826/193 |
| STAT4 | NM_003151 | 361 & 362 | 222/46 | 981/127 | 228/15 |
| STAT2 | NM_005419 | 363 & 364 | 300/50 | 876/220 | 428/40 |
| musculin | NM_005098 | 365 & 366 | 148/53 | 1133/162 | 468/90 |
| CTNND1 | AF062328 | 367 & 368 | 315/60 | 902/158 | 500/120 |
| H-plk | NM_015852 | 369 & 370 | 169/26 | 281/19 | 93/36 |
| ATF5 | NM_012068 | 371 & 372 | 1001/151 | 3757/333 | 2513/31 |
| AP-2 alpha | NM_003220 | 373 & 374 | 193/135 | 613/37 | 166/14 |
| c-maf | AF055376 | 375 & 376 | 35/31 | 181/73 | 88/43 |
| TR2 | M21985 | 377 & 378 | 69/36 | 218/43 | 100/75 |
| NFE2L3 | NM_004289 | 379 & 380 | 180/57 | 505/150 | 206/29 |
| ORC5T | AF081459 | 381 & 382 | 165/85 | 259/27 | 130/43 |
| MGC:2268 | BC000080 | 383 & 384 | 39/19 | 811/103 | 85/19 |
| BST2 | NM_004335 | 385 & 386 | 1168/122 | 3885/427 | 1885/122 |
| cig5 | AF026941 | 387 & 388 | 61/56 | 543/71 | 113/34 |
| FEZ1 | NM_005103 | 389 & 390 | 178/52 | 2575/127 | 1079/158 |
| Pirin | NM_003662 | 391 & 392 | 155/70 | 562/61 | 291/17 |
| G0S2 | NM_015714 | 393 & 394 | 475/111 | 1235/256 | 688/134 |
| HSPA6 | NM_002155 | 395 & 396 | 323/184 | 1023/275 | 281/211 |
| MGC:13087 | BC006141 | 397 & 398 | 96/67 | 357/57 | 186/8 |
| HIG2 | NM_013332 | 399 & 400 | 790/39 | 1459/138 | 421/120 |
| protease inhibitor 15 | NM_015886 | 401 & 402 | 88/79 | 268/42 | 84/20 |
| SCO2 | NM_005138 | 403 & 404 | 799/129 | 2014/483 | 1193/163 |
| MDA5 | NM_022168 | 405 & 406 | 187/80 | 784/161 | 413/69 |
| PROL2 | NM_006813 | 87 & 88 | 402/94 | 890/140 | 389/188 |
| MGC:12814 | BC006101 | 407 & 408 | 75/44 | 705/42 | 327/71 |
| AD7C-NTP | NM_014486 | 409 & 410 | 150/23 | 373/96 | 178/78 |
| TFPI | NM_006287 | 411 & 412 | 40/27 | 148/6 | 28/32 |
| TTY1 | AF000990 | 413 & 414 | 119/15 | 308/21 | 209/70 |
| FKSG18 | AF317129 | 415 & 416 | 480/83 | 1294/262 | 645/260 |
| FGL2 | NM_006682 | 417 & 418 | 81/93 | 535/191 | 311/25 |
| laforin | BC005286 | 419 & 420 | 86/74 | 267/47 | 96/48 |
| MGC:10978 | BC004395 | 421 & 422 | 134/122 | 475/79 | 241/152 |
| SUPAR | AY029180 | 423 & 424 | 500/118 | 3118/344 | 2105/298 |
| gp130-RAPS | AB015706 | 425 & 426 | 101/58 | 243/65 | 150/25 |
| DBCCR1 | NM_014618 | 427 & 428 | 19/7 | 163/41 | 63/70 |
| CRIM1 | NM_016441 | 429 & 430 | 36/47 | 370/102 | 174/77 |
| MGC:4655 | BC004908 | 431 & 432 | 1229/35 | 3256/517 | 1692/137 |
| FLJ23231 | NM_025079 | 127 & 128 | 209/66 | 1466/38 | 498/122 |
| FLJ12806 | NM_022831 | 433 & 434 | 201/60 | 360/18 | 194/71 |
| FLJ10134 | NM_018004 | 435 & 436 | 420/12 | 1761/322 | 802/50 |
| UXS1 | NM_025076 | 437 & 438 | 810/61 | 3147/132 | 1551/218 |
| FLJ22341 | NM_024599 | 439 & 440 | 171/98 | 530/129 | 276/55 |
| NAV3 | NM_014903 | 125 & 126 | 811/146 | 2176/246 | 482/77 |
| FLJ00048 | AK024456 | 441 & 442 | 139/79 | 473/129 | 311/18 |
| IMAGE:4128465 | BC007843 | 443 | 1056/88 | 4314/987 | 2023/272 |
| MGC:9246 | BC009699 | 444 & 445 | 6313/536 | 14679/2127 | 5196/641 |
| Oligodendrocyte | AK091462 | 446 & 447 | 162/44 | 793/40 | 320/150 |
| lineage transcription | | | | | |
| factor 2 | | | | | |
| FLJ23535 | AK027188 | 448 & 449 | 324/74 | 2409/58 | 768/124 |
| neutrophil cytosolic | BC002816 | 450 & 451 | 630/28 | 4612/235 | 3418/58 |
| factor 1 | | | | | |
| FLJ11259 | NM_018370 | 452 & 453 | 340/115 | 1634/76 | 479/160 |
| KIAA0084 | D42043 | 454 & 455 | 631/22 | 1711/177 | 660/119 |
| FLJ20637 | NM_017912 | 456 & 457 | 142/162 | 886/112 | 218/91 |
| FLJ37747 | AK095066 | 458 | 184/16 | 402/48 | 210/38 |
| KIAA0937 | AB023154 | 459 & 460 | 242/82 | 2065/564 | 540/98 |
| FLJ21175 | AK024828 | 461 & 462 | 207/48 | 637/70 | 136/104 |
| HSPC177 | NM_016410 | 463 & 464 | 624/177 | 1847/70 | 806/256 |
| FLJ23094 | AK026747 | 465 & 466 | 404/226 | 2434/192 | 796/85 |
| FLJ13054 | AK023116 | 467 & 468 | 369/26 | 858/214 | 425/58 |
| FLJ22693 | NM_022750 | 469 & 470 | 488/54 | 2037/266 | 800/64 |
| KIAA0856 | AB020663 | 471 & 472 | 302/59 | 1072/102 | 323/167 |
| PRO2870 | AF130080 | 473 & 474 | 364/115 | 1879/448 | 691/233 |
| MGC5347 | NM_024063 | 475 & 476 | 245/38 | 482/43 | 223/123 |
| KIAA0984 | AB023201 | 477 & 478 | 186/65 | 340/22 | 75/19 |
| FLJ10901 | NM_018265 | 479 & 480 | 889/139 | 2586/309 | 1261/248 |
| FLJ13397 | NM_024948 | 481 & 482 | 46/34 | 244/74 | 50/27 |
| FLJ20035 | NM_017631 | 483 & 484 | 158/62 | 538/151 | 248/57 |
| KIAA0286 | AB006624 | 485 & 486 | 158/12 | 417/124 | 236/15 |
| KIAA0247 | NM_014734 | 487 & 488 | 732/87 | 2473/484 | 1326/299 |
| cyld | AJ250014 | 489 & 490 | 333/87 | 1383/112 | 864/254 |
| FLJ11286 | NM_018381 | 491 & 492 | 393/42 | 1714/376 | 998/303 |
| FLJ20073 | NM_017654 | 493 & 494 | 106/11 | 417/74 | 254/91 |
| FLJ10111 | NM_017999 | 495 & 496 | 145/34 | 461/93 | 305/48 |
| UXS1 | NM_025076 | 437 & 438 | 810/61 | 3147/132 | 1551/218 |
| PLAC8 | NM_016619 | 497 & 498 | 8906/420 | 12175/736 | 2981/61 |
| KIAA0987 | NM_012307 | 499 & 500 | 58/83 | 293/42 | 102/43 |
| FLJ20234 | BC000795 | 501 & 502 | 219/87 | 497/172 | 157/8 |
| FLJ10849 | NM_018243 | 503 & 504 | 160/60 | 290/87 | 122/16 |
| DKFZp434F0318 | NM_030817 | 505 & 506 | 68/35 | 141/15 | 43/27 |
| FLJ22800 | NM_024795 | 507 & 508 | 61/68 | 162/32 | 69/43 |
| KIAA0805 | AB018348 | 509 & 510 | 75/48 | 314/39 | 143/100 |
| MGC11335 | NM_030819 | 511 & 512 | 141/27 | 291/79 | 117/101 |
| FLJ20651 | NM_017919 | 513 & 514 | 94/69 | 287/104 | 127/106 |
| FLJ13105 | NM_025001 | 515 & 516 | 79/44 | 197/62 | 112/42 |
| KIAA1005 | AB023222 | 517 & 518 | 29/4 | 116/50 | 40/16 |
| FLJ23191 | NM_024574 | 519 & 520 | 160/44 | 313/91 | 141/95 |
| KIAA0671 | AB014571 | 521 & 522 | 171/148 | 340/93 | 121/33 |
| FLJ23231 | NM_025079 | 127 & 128 | 209/66 | 1466/141 | 498/122 |
| IMAGE:4718024 | BC022281 | 523 & 524 | 187/37 | 1321/236 | 452/92 |
| FLJ00055 | AK024462 | 525 & 526 | 250/11 | 1204/129 | 623/22 |
| IMAGE:4447884 | BC020595 | 527 & 528 | 355/32 | 1170/141 | 701/65 |
| DKFZp586C091 | AL050119 | 529 | 103/66 | 268/93 | 147/14 |
| FLJ40021 | AK097340 | 530 & 531 | 15/19 | 102/27 | 13/8 |
| FLJ36863 | AK094182 | 532 | 100/37 | 161/15 | 19/12 |
| MGC:4637 | BC005879 | 533 & 534 | 200/46 | 294/65 | 83/68 |
| STAT1 | NM_139266 | 823 & 748 | 244/136 | 4092/654 | 1066/221 |

Values represent averages and standard deviations of normnalized expression values for three replicates per group.

TABLE 5
Genes whose expression is induced after 2 hours in the presence
of the NFκB inhibitor BMS-205820

| | | | | | LPS/BMS- |
|---|---|---|---|---|---|
| Gene | Accession # | Seq ID Nos. | Unstimulated | LPs | 205820 |
| ID2 | NM_002166 | 535 & 536 | 2218/168 | 711/171 | 2004/558 |
| MEF2D | NM_005920 | 537 & 538 | 906/91 | 366/30 | 708/120 |
| retinoic acid receptor, | NM_000964 | 539 & 540 | 752/152 | 174/76 | 539/137 |
| alpha | | | | | |
| RUNX3 | NM_004350 | 541 & 542 | 1887/326 | 592/123 | 1203/41 |
| CALIFp | AF180476 | 543 & 544 | 570/24 | 217/29 | 405/69 |
| MAFB | NM_005461 | 545 & 546 | 226/73 | 379/102 | 516/88 |
| RREB1 | NM_002955 | 547 & 548 | 1134/216 | 460/106 | 966/84 |
| beta-glucocorticoid | X03348 | 549 & 550 | 278/86 | 132/66 | 300/80 |
| receptor | | | | | |
| HEX gene | Z21533 | 551 & 552 | 191/79 | 48/31 | 132/5 |
| LTBP3 | NM_021070 | 553 & 554 | 132/60 | 37/45 | 180/48 |
| TXNIP | NM_006472 | 555 & 556 | 9899/1323 | 2560/322 | 5187/987 |
| Similar to LIM domain | BC003096 | 557 & 558 | 431/140 | 238/134 | 721/57 |
| protein | | | | | |
| SQSTM1 | NM_003900 | 559 & 560 | 1330/36 | 3229/760 | 4869/460 |
| RGS12 | AF030110 | 561 & 562 | 272/39 | 15/10 | 305/94 |
| SH3GL1 | NM_003025 | 563 & 564 | 1205/143 | 416/284 | 964/105 |
| type II cAMP- | M90360 | 565 & 566 | 242/40 | 123/79 | 341/63 |
| dependent protein | | | | | |
| kinase | | | | | |
| TESK1 | NM_006285 | 567 & 568 | 474/124 | 132/73 | 329/85 |
| PRDX2 | NM_005809 | 569 & 570 | 368/20 | 146/27 | 377/63 |
| NADPH-cytochrome | AF258341 | 571 & 572 | 379/85 | 152/78 | 377/88 |
| P450 reductase | | | | | |
| CYP1A2 | NM_000761 | 573 & 574 | 194/31 | 69/39 | 284/140 |
| kallikrein 13 | NM_015596 | 575 & 576 | 272/59 | 145/14 | 310/56 |
| LCAT-like | AB017494 | 577 & 578 | 316/51 | 141/50 | 248/9 |
| lysophospholipase | | | | | |
| histidyl-tRNA | U18937 | 579 & 580 | 752/114 | 306/11 | 593/127 |
| synthetase homolog | | | | | |
| CCR1 | NM_001295 | 581 & 582 | 1433/194 | 264/55 | 1128/55 |
| TNFRSF1A | NM_001065 | 583 & 584 | 1976/224 | 546/137 | 1557/186 |
| P2Y5 | NM_005767 | 585 & 586 | 179/14 | 89/16 | 201/42 |
| SLC17A5 | NM_012434 | 587 & 588 | 1630/40 | 785/140 | 2058/403 |
| KCNN4 | NM_002250 | 589 & 590 | 1559/259 | 609/122 | 2542/246 |
| TNFSF14 | NM_003807 | 591 & 592 | 794/111 | 378/120 | 1232/198 |
| SFD alpha | AF112204 | 593 & 594 | 920/170 | 422/8 | 629/168 |
| thromboxane A2 | D38081 | 595 & 596 | 317/36 | 130/41 | 313/32 |
| receptor | | | | | |
| GABRR1 | NM_002042 | 597 & 598 | 137/39 | 36/6 | 183/49 |
| adenosine A3 receptor | NM_000677 | 599 & 600 | 230/85 | 59/43 | 165/26 |
| integrin, alpha 5 | NM_002205 | 601 & 602 | 3492/266 | 1720/289 | 4459/171 |
| sodium bicarbonate | AF069510 | 603 & 604 | 341/35 | 95/72 | 224/73 |
| cotransporter | | | | | |
| amelogenin | NM_001142 | 605 & 606 | 146/21 | 42/9 | 160/80 |
| HEC | NM_006101 | 607 & 608 | 370/65 | 154/35 | 311/22 |
| ALTE | NM_004729 | 609 & 610 | 912/205 | 300/156 | 572/80 |
| HCG II | X81001 | 611 | 423/34 | 168/178 | 388/44 |
| MAD1L1 | NM_003550 | 612 & 613 | 540/60 | 159/15 | 442/125 |
| MAP1B | NM_005909 | 614 & 615 | 97/16 | 37/51 | 148/23 |
| pelota homolog | NM_015946 | 616 & 617 | 326/46 | 177/73 | 341/101 |
| ICB-1 | NM_004848 | 618 & 619 | 646/82 | 196/106 | 529/186 |
| Similar to CAP-binding | BC022786 | 620 & 621 | 1042/47 | 399/44 | 709/85 |
| protein complex | | | | | |
| interacting protein 2 | | | | | |
| IMAGE:3939659 | BC012778 | 622 & 623 | 720/71 | 234/35 | 706/100 |
| FLJ00216 | AK074143 | 624 & 625 | 822/132 | 367/105 | 777/83 |
| TRIP-Br2 | NM_014755 | 626 & 627 | 585/99 | 255/69 | 692/80 |
| FLJ13479 | NM_024706 | 628 & 629 | 248/43 | 42/44 | 194/48 |
| KIAA0241 | D87682 | 630 & 631 | 517/136 | 290/75 | 552/10 |
| MGC5338 | NM_024062 | 632 & 633 | 276/81 | 63/43 | 211/27 |
| KIAA0349 | AB002347 | 634 & 635 | 256/92 | 113/63 | 284/39 |
| PRO1048 | NM_018497 | 636 & 637 | 124/12 | 34/26 | 190/41 |
| clone 161455 | U66046 | 638 | 177/61 | 91/20 | 217/15 |
| MGC:33567 | BC038297 | 639 & 640 | 856/241 | 326/29 | 724/167 |
| MGC:23591 | BC015781 | 641 & 642 | 210/38 | 84/64 | 258/38 |
| C20orf172 | NM_024918 | 643 & 644 | 236/63 | 116/2 | 264/56 |
| DKFZp667O2416 | AL512765 | 645 & 646 | 320/70 | 157/38 | 370/56 |
| ZFP64 | NM_018197 | 647 & 648 | 280/3 | 98/53 | 194/50 |
| FUS glycine rich | X71428 | 649 & 650 | 123/34 | 69/12 | 166/41 |
| protein | | | | | |
| FLJ23420 | NM_025061 | 651 & 652 | 129/50 | 12/7 | 146/84 |
| MGC:13138 | BC008821 | 653 & 654 | 244/22 | 117/60 | 297/81 |
| FLJ13119 | NM_024580 | 655 & 656 | 443/48 | 159/22 | 278/39 |
| DKFZP564O0823 | NM_015393 | 657 & 658 | 339/73 | 158/64 | 354/123 |

Values represent averages and standard deviations of normalized expression values for three replicates per group.

TABLE 6
Genes whose expression is induced after 8 hours in the presence
of the the NFκB inhibitor BMS-205820

| | | | | | LPS/BMS- |
|---|---|---|---|---|---|
| Gene | Accession # | Seq ID Nos. | Unstimulated | LPs | 205820 |
| MRG1 | AF109161 | 659 & 660 | 1470/186 | 695/86 | 1514/164 |
| RNF24 | NM_007219 | 661 & 662 | 1111/114 | 427/22 | 870/46 |
| PEX6 | NM_000287 | 663 & 664 | 284/25 | 154/157 | 377/109 |
| GLUT3 | M20681 | 665 & 666 | 110/47 | 36/21 | 184/8 |
| mitochondrial solute | AF155660 | 667 & 668 | 275/14 | 105/87 | 201/30 |
| carrier | | | | | |
| CDK5 | NM_004935 | 669 & 670 | 588/102 | 270/140 | 615/206 |
| synaptojanin 2 | AF318616 | 671 & 672 | 234/110 | 52/20 | 215/56 |
| lysophospholipase-like, | BC006230 | 673 & 674 | 397/71 | 1414/38 | 1981/395 |
| IRS2 | NM_003749 | 675 & 676 | 2614/198 | 900/143 | 1448/554 |

Values represent averages and standard deviations of normalized expression values for three replicates per group.

### Example 5

### Method of Confirming the Functional Relevance of the NF-κB Pathway-Associated Polynuceotides and Polypeptides to the NFκB Pathway Through the Application of Antisense Oligonucleotide Methodology

Human microvascular endothelial cells (HMVEC, Clonetics, Walkersville, Md.) were plated in 48 well tissue culture plates at 30,000 cells per well and cultured overnight in EGM-2 medium (Clonetics) at 37° C. in 5% CO₂. The next morning, the cells were transfected with 25 nM oligomer and 0.75 ug/ml lipofectamine 2000 (Invitrogen). Following an overnight culture with oligomers, the cells were stimulated with 10 ng/ml TNFα for 6 hrs and analyzed for E-selecting expression by ELISA. Expression was normalized to cell number. Antisense oligomers selective for NF-κB target genes BCL-6 and DGCRK6 significantly inhibited TNFα-induced E-selecting expression. This inhibition was equivalent to, or greater than that achieved using antisense oligomers specific for IKK-2. These data suggest that BCL-6 and DGCRK6 are functionally linked to an NF-κB dependent response.

### Example 6

### Method of Determining Alteration in a Gene Corresponding to the NF-κB Pathway-Associated Polynucleotides

RNA isolated from entire families or individual patients presenting with a phenotype of interest (such as a disease) is isolated. cDNA is then generated from these RNA samples using protocols known in the art. (See, e.g., J. Sambrook et al., Molecular Cloning: A Laboratory Manual, Cold Spring Harbor Laboratory, Cold Spring Harbor, N.Y., 1989). The cDNA is used as a template for PCR, employing primers surrounding the regions of interest in (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823).

Suggested PCR conditions consist of 35 cycles at 95° C. for 30 seconds; 60-120 seconds at 52° C.-58° C.; and 60-120 seconds at 70° C., using buffer solutions described, for example, in Sidransky et al., 1991, Science, 252:706.

PCR products are then sequenced using primers labeled at their 5′ end with T4 polynucleotide kinase, employing SequiTherm Polymerase. (Epicentre Technologies, Madison, Wis.). The intron-exon borders of selected exons is also determined and genomic PCR products analyzed to confirm the results. PCR products harboring suspected mutations are then cloned and sequenced to validate the results of the direct sequencing. PCR products are cloned into T-tailed vectors as described in Holton et al., 1991, Nucleic Acids Research, 19:1156 and are sequenced with T7 polymerase (United States Biochemical). Affected individuals are identified by mutations not present in unaffected individuals.

Genomic rearrangements also serve as a method of determining alterations in a gene corresponding to a polynucleotide. Genomic clones are nick-translated with digoxigenindeoxy-uridine 5′-triphosphate (Boehringer Manheim), and FISH is performed as described in Johnson et al., 191, Methods Cell Biol., 35:73-99. Hybridization with the labeled probe is carried out using a vast excess of human cot-1 DNA for specific hybridization to the corresponding genomic locus. Chromosomes are counter stained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson et al., 1991, Genet. Anal. Tech. Appl., 8:75). Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as diagnostic markers for an associated disease.

### Example 7

### Alternative Methods of Detecting Polymophisms in the NF-κB Pathway-Associated Polynucleotides

Preparation of Samples: Polymorphisms are detected in a target nucleic acid from an individual being analyzed. To assay genomic DNA, virtually any biological sample (other than pure red blood cells) is suitable. For example, convenient tissue samples include whole blood, semen, saliva, tears, urine, fecal material, sweat, buccal, skin and hair. To assay cDNA or mRNA , the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by methods known in the art, particularly, for example, PCR. See generally, PCR Technology: Principles and Applications for DNA Amplification, (ed.) H. A. Erlich, Freeman Press, New York, N.Y., 1992; PCR Protocols: A Guide to Methods and Applications (eds.) Innis, et al., Academic Press, San Diego, Calif., 1990); Mattila et al., 1991, Nucleic Acids Res., 19: 4967; Eckert et al., 1991, PCR Methods and Applications 1; PCR (eds.) McPherson et al., IRL Press, Oxford; and U.S. Pat. No. 4,683,202. Other suitable amplification methods include the ligase chain reaction (LCR) (See, e.g., Wu and Wallace, 1989, Genomics, 4:560; Landegren et al., 1988, Science, 241:1077); transcription amplification (Kwoh et al., 1989, Proc. Natl. Acad Sci. USA, 86:1173); self-sustained sequence replication (Guatelli et al., 1990, Proc. Nat. Acad Sci. USA, 87:1874); and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively. Additional methods of amplification are known in the art or are described elsewhere herein.

Detection of Polymorphisms in Target DNA: There are two distinct types of analyses of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals with identify points of variation, i.e., polymorphic sites. By analyzing groups of individuals representing the greatest ethnic diversity among humans, and the greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described further herein.

The second type of analysis determines which form(s) of a characterized (known) polymorphism are present in individuals undergoing testing. Additional methods of analysis are known in the art or are described elsewhere herein.

Allele-Specific Probes: The design and use of allele-specific probes for analyzing polymorphisms is described, for example, by Saiki et al., 1986, Nature, 324:163-166; Dattagupta, EP 235,726; and Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, in which a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This type of probe design achieves good discrimination in hybridization between different allelic forms. Allele-specific probes are often used in pairs, with one member of the pair showing a perfect match to a reference form of a target sequence and the other member showing a perfect match to a variant form. Several pairs of probes can then be immobilized on the same support for simultaneous analysis of multiple polymorphisms within the same target sequence.

Tiling Arrays: Polymorphisms can also be identified by hybridization to nucleic acid arrays, some examples of which are described in WO 95/11995. The same arrays, or different arrays, can be used for the analysis of characterized polymorphisms. WO 95/11995 also describes sub-arrays that are optimized for the detection of a variant form of a pre-characterized polymorphism. Such a sub-array contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 20 or more bases).

Allele-Specific Primers: An allele-specific primer hybridizes to a site on target DNA overlapping a polymorphism and only primes the amplification of an allelic form to which the primer exhibits perfect complementarity. See, e.g., Gibbs, 1989, Nucleic Acid Res., 17:2427-2448. An allele-specific primer is used in conjunction with a second primer which hybridizes at a distal site. Amplification proceeds from the two primers, resulting in a detectable product which indicates that the particular allelic form is present. A control is usually performed with a second pair of primers, one of which shows a single base mismatch at the polymorphic site, and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., WO 93/22456).

Direct-Sequencing: The direct analysis of the sequence of NF-κB pathway-associated polynucleotides polymorphisms according to this invention can be accomplished using either the dideoxy chain termination method, or the Maxam—Gilbert method (see, e.g., J. Sambrook et al., Molecular Cloning, A Laboratory Manual (2nd Ed., CSHP, New York 1989); and Zyskind et al., 1988, Recombinant DNA Laboratory Manual, (Acad. Press).

Denaturing Gradient Gel Electrophoresis: Amplification products generated using the polymerase chain reaction can be analyzed by the use of denaturing gradient gel electrophoresis. Different alleles can be identified based on the different sequence-dependent melting properties and the electrophoretic migration of DNA in solution. (e.g., Chapter 7, PCR Technology. Principles and Applications for DNA Amplification, (ed.) Erlich, W.H. Freeman and Co, New York, 1992).

Single-Strand Conformation Polymorphism Analysis: Alleles of target sequences can be differentiated using single-strand conformation polymorphism analysis, which identifies base differences by alteration in electrophoretic migration of single stranded PCR products, as described in Orita et al., 1989, Proc. Nat. Acad. Sci. USA, 86:2766-2770. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

Single Base Extension: An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., 1997, Proc. Natl. Acad. Sci. USA, 94:10756-61, uses a locus-specific oligonucleotide primer labeled on the 5′ terminus with 5-carboxyfluorescein (F AM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 8

### Method of Genotyping Each NF-κB Pathway-Associated Polynucleotide SNP

Genomic DNA preparation: Genomic DNA samples for genotyping are prepared using the Purigene™ DNA extraction kit from Gentra Systems (Gentra Systems, Inc., Minneapolis, Minn.). After preparation, DNA samples are diluted to a 2 ng/ul working concentration with TE buffer (10 mM Tris-Cl, pH 8.0, 0.1 mM EDTA, pH 8.0) and stored in 1 ml 96 deep-well plates (VWR) at −20° C. until use. Samples for genomic DNA preparation may be obtained from the Coriell Institute (Collingswood, N.J.), patients participating in a Bristol-Myers Squibb (BMS) clinical study, from other sources known in the art, or as otherwise described herein.

Genotyping: SNP genotyping reactions are performed using the SNPStream™ system (Orchid Biosience, Princeton, N.J.) based on genetic bit analysis (T. Nikiforov et al., 1994, Nucl. Acids Res., 22:4167-4175). The regions including 5polymorphic sites are amplified by PCR using a pair of primers (OPERON Technologies), one of which can be phosphorothioated. 6 μl of a PCR cocktail containing 1.0 ng/μl of genomic DNA, 200 μM dNTPs, 0.5 μM forward PCR primer, 0.5 μM reverse PCR primer (phosphorothioated), 0.05 U/μl Platinum Taq DNA polymerase (LifeTechnologies), and 1.5 mM MgCl₂. The PCR primer pairs used for genotyping analysis can be designed using methods known in the art in conjunction with the teachings described herein.

PCR reactions are set up in 384-well plates (MJ Research) using a MiniTrak liquid handling station (Packard Bioscience). PCR thermocycling can be performed under the following conditions in a MJ Research Tetrad machine: step1, 95 degrees for 2 min; step 2, 94 degrees for 30 min; step 3, 55 degrees for 2 min; step 4, 72 degrees for 30 sec; step 5, go back to step 2 for an additional 39 cycles; step 6, 72 degrees for 1 min; and step 7, 12 degrees indefinitely. After thermocycling, the amplified samples are placed in the SNPStream™ (Orchid Bioscience) machine, and the automated genetic bit analysis (GBA) reaction (T. Nikiforov et al., Ibid.) is performed. The first step of this reaction involves degradation of one of the strands of the PCR products by T7 gene 6 exonuclease to yield single-stranded products. The strand containing phosphorothioated primer is resistant to T7 gene 6 nuclease, and is not degraded by this enzyme. After digestion, the single-stranded PCR products are subjected to an annealing step in which the single stranded PCR products are annealed to the GBA primer on a solid phase, and then subjected to the GBA reaction (single base extension) using dideoxy-NTPs labeled with biotin or fluorescein. The GBA primers are designed using methods known in the art, in conjunction with the teachings of the present invention.

The present invention encompasses the substitution of certain polynucleotides within the GBA primers with a polynucleotide that can be substituted with a C3 linker (C3 spacer phosphoramidite) during synthesis of the primer. Such linkers can be obtained from Research Genetics; Sigma-Genosys; or Operon, for example. Incorporation of the dideoxynucleotides into a GBA primer is detected by use of a two color ELISA assay using anti-fluorescein alkaline phosphatase conjugate and anti-biotin horseradish peroxidase antibodies. Automated genotype calls are made by GenoPak software (Orchid Bioscience). Manual correction of automated calls can be performed upon inspection of the resulting allelogram of each SNP.

### Example 9

### Alternative Method of Genotypying NF-κB Pathway-Associated Polynucleotides SNPs

In addition to the method of genotyping described herein above, the skilled artisan can determine the genotype of the NF-κB pathway-associated polynucleotides polymorphisms of the present invention using the below described alternative method. This method is referred to as the “GBS method” herein and can be performed as described in conjunction with the teachings as described elsewhere herein.

Briefly, the direct analysis of the sequence of NF-κB pathway-associated polynucleotides polymorphisms of the present invention is accomplished by DNA sequencing of PCR products corresponding to the same PCR amplicons that are designed to be in close proximity to the polymorphisms of the present invention using the Primer3 program. The M13_{—SEQUENCE}1 “tgtaaaacgacggccagt”, (SEQ ID NO: 745), is prepended to each forward PCR primer. The M13_SEQUENCE2 “caggaaacagctatgacc”, (SEQ ID NO: 746), is prepended to each reverse PCR primer. Each forward and reverse primer is based upon the coding region of the region flanking the SNP and is designed such that the SNP is amplified.

PCR amplification can be performed on genomic DNA samples amplified from (20 ng) in reactions (50 μl) containing 10 mM Tris-Cl pH 8.3, 50 mM KCl, 2.5 mM MgCl₂, 150 μM dNTPs, 3 μM PCR primers, and 3.75 U TaqGold DNA polymerase (PE Biosystems). PCR can be performed in MJ Research Tetrad machines under a set of cycling conditions comprising 94° C., 10 minutes, 30 cycles of 94° C., 30 seconds, 60° C., 30 seconds, and 72° C., 30 seconds, followed by 72° C., 7 minutes. PCR products are purified using QIAquick PCR purification kit (Qiagen) and are sequenced by the dye-terminator method using PRISM 3700 automated DNA sequencer (Applied Biosystems, Foster City, Calif.) following the manufacturer's instruction outlined in the Owner's Manual, which is hereby incorporated herein by reference in its entirety. PCR products are sequenced by the dye-terminator method using the M13_SEQUENCE1 (SEQ ID NO: 745) and M13_SEQUENCE2 (SEQ ID NO: 746) primers as described above. The genotype can be determined by analysis of the sequencing results at the polymorphic position.

### Example 10

### Additional Methods of Genotyping NF-κB Pathway-Associated Polynucleotides SNPs

The skilled practitioner appreciates that there are a number of methods suitable for genotyping a SNP of the present invention, aside from the preferred methods described herein. The present invention encompasses the following non-limiting types of genotype assays: PCR-free genotyping methods; Single-step homogeneous methods; Homogeneous detection with fluorescence polarization; Pyrosequencing; “Tag” based DNA chip system; Bead-based methods; fluorescent dye chemistry; Mass spectrometry based genotyping assays; TaqMan genotype assays; Invader genotype assays; and microfluidic genotype assays, among others. Also encompassed by the present invention are the following, non-limiting genotyping methods: U. Landegren et al., 1998, Genome Res. 8:769-776; P. Kwok, 2000, Pharmacogenomics, 1:95-100; I. Gut, 2001, Hum Mutat., 17:475-492; D. Whitcombe et al., 1998, Curr. Opin. Biotechnol., 9:602-608; S. Tillib and A. Mirzabekov, 2001, Curr. Opin. Biotechnol., 12:53-58; E. Winzeler et al., 1998, Science, 281:1194-1197; V. Lyamichev et al., 1999, Nat. Biotechnol., 17:292-296; J. Hall et al., 2000, Proc. Natl. Acad. Sci. USA, 97:8272-8277; C. Mein et al., 2000, Genome Res., 10:333-343; Y. Ohnishi et al., 2001, J. Hum. Genet., 46: 471-477; M. Nilsson et al., 1994, Science, 265:2085-2088; J. Baner et al., 1998, Nucleic Acids Res., 26: 5073-5078; J. Baner et al., 2001, Curr. Opin. Biotechnol., 12:11-15; A. Hatch et al., 1999, Genet. Anal., 15:35-40; P. Lizardi et al., 1998, Nat. Genet., 19(3):225-232; X. Zhong et al., 2001, Proc. Natl. Acad Sci. USA, 98:3940-3945; F. Faruqi et al., 2001, BMC Genomics 2, 4; K. Livak, 1999, Genet. Anal., 14:143-149; S. Marras et al., 1999, Genet. Anal., 14:151-156; K. Ranade et al., 2001, Genome Res., 11:1262-1268; M. Myakishev et al., 2001, Genome Res., 11:163-169; L. Beaudet et al., 2001, Genome Res., 11:600-608; X. Chen et al., 1999, Genome Res., 9:492-498; N. Gibson et al., 1997, Clin. Chem., 43:1336-1341; S. Latif et al., 2001, Genome Res., 11: 436-440; T. Hsu et al., 2001, Clin. Chem., 47:1373-1377; A. Alderbom et al., 2000, Genome Res., 10:1249-1258; M. Ronaghi et al., 1998, Science, 281:363, 365; M. Ronaghi, 2001, Genome Res., 11:3-11; A. Pease et al., 1994, Proc. Natl. Acad. Sci. USA, 91:5022-5026; E. Southern et al., 1993, Genomics, 13:1008-1017; D. Wang et al., 1998, Science, 280:1077-1082; P. Brown and D. Botstein, 1999, Nat. Genet., 21:33-37; M. Cargill et al., 1999, Nat. Genet., 22:231-238; S. Dong et al., 2001, Genome Res., 11:1418-1424; M. Halushka et al., 1999, Nat. Genet., 22:239-247; J. Hacia, 1999, Nat. Genet., 21:42-47; R. Lipshutz et al., 1999, Nat. Genet., 21:20-24; R. Sapolsky et al., 1999, Genet. Anal., 14:187-192; Z. Tsuchihashi and P. Brown, 1994, J. Virol., 68:5863; D. Herschlag, 1995, J. Biol. Chem., 270:20871-20874; S. Head et al., 1997, Nucleic Acids Res., 25:5065-5071; T. Nikiforov et al., 1994, Nucleic Acids Res., 22:4167-4175; A. Syvanen et al., 1992, Genomics, 12:590-595; J. Shumaker et al., 1996, Hum Mutat, 7:346-354; K. Lindroos et al., 2001, Nucleic Acids Res., 29:E69-9; K. Lindblad-Toh et al., 2000, Nat. Genet., 24:381-386; T. Pastinen et al., 2000, Genome Res., 10:1031-1042; J. Fan et al., 2000, Genome Res, 10:853-860; J. Hirschhorn et al., 2000, Proc. Natl. Acad Sci. USA, 97:12164-12169; A. Bouchie, 2001, Nature Biotechnol., 19:704; M. Hensel et al., 1995, Science, 269:400-403; D. Shoemaker et al., 1996, Nature Genet., 14:450-456; N. Gerry et al., 1999, J. Mol. Biol., 292:251-262; D. Ladner et al., 2001, Lab. Invest., 81:1079-1086; M. Iannone et al., 2000, Cytometry, 39:131-140; R. Fulton et al., 1997, J. Clin. Chem., 43:1749-1756; B. Armstrong et al., 2000, Cytometry, 40:102-108 H. Cai et al., 2000, Genomics, 69:395; J. Chen et al., 2000, Genome Res., 10:549-557; F. Ye et al., 2001, Hum Mutat., 17:305-316; K. Michael et al., 1998, Anal. Chem., 70:1242-1248; F. Steemers et al., 2000, Nature Biotechnol., 18:91-94;W. Chan and S. Nie, 1998, Science, 281:2016-2018; M. Han et al., 2001, Nature Biotechnol., 19:631-635; T. Griffin and L. Smith, 2000, Trends Biotechnol., 18:77-84; P. Jackson et al., 2000, Mol. Med. Today, 6:271-276; L. Haff and I. Smirnov, 1997, Genome Res., 7:378-388; P. Ross et al., 1998, Nat. Biotechnol., 16:1347-1351; M. Bray et al., 2001, Hum. Mutat., 17:296-304; S. Sauer et al., 2000, Nucleic Acids Res., 28:E13; S. Sauer et al., 2000, Nucleic Acids Res., 28:E100; X. Sun et al., 2000, Nucleic Acids Res., 28:E68; K. Tang et al., 1999, Proc. Natl. Acad. Sci. USA, 91:10016-10020; J. Li et al., 1999, Electrophoresis, 20:1258-1265; D. Little et al., 1997, Nat. Med., 3:1413-1416; D. Little et al., 1997, Anal. Chem., 69:4540-4546; T. Griffin et al. 1997, Nat. Biotechnol., 15:1368-1372; P. Ross et al., 1997, Anal. Chem., 69:4197-4202; P. Jiang-Baucom et al., 1997, Anal. Chem., 69:4894-4898; T. Griffin et al., 1999, Proc. Natl. Acad Sci USA, 96:6301-6306; M. Kokoris et al., 2000, Mol. Diagnos., 5:329-340; C. Jurinke et al., 2001, Methods Mol. Biol., 200:170:103-16; and/or N. Taranenko et al., 1996, Genet. Anal., 13:87-94.

### Example 11

### Use of Other NF-κB Inhibitors and NF-κB Knock-Out Cell Lines to Confirm the Regulation of Selected Target Genes

### TaqMan Analyses

PolyA+ mRNA was isolated from THP1 cells that were either unstimulated, stimulated with LPS for 2 hours, or stimulated with LPS for 2 hours in the presence of the peptide BMS-205820 (2 μM). In some experiments, THP-1 cells were stimulated with LPS in the presence of the glucocorticoid dexamethasone (100 nM), and the IKK-2 inhibitor, BMS-345541 (10 μM). RNA quality and quantity were evaluated using UV spectrometry and capillary electrophoresis with the RNA 6000 Assay by Agilent. Five-hundred nanograms of polyA RNA was used for first-strand cDNA synthesis using the SuperScript™ First-Strand Synthesis System for RT-PCR (Life Technologies) following the manufacturer's instructions with 250 ng of random hexamers.

For the NF-κB knockout studies, wild type 3T3 cells, 3T3 fusions of embryonic fibroblasts derived from p65 and IκBα knockouts, or embryonic fibroblasts derived from p50 and RelB knockouts were stimulated for 2 and 8 hours with 10 ng/ml TNFα or 10 ng/ml PMA. RNA isolation and cDNA synthesis were performed as described above.

PCR Reactions were performed in a total volume of 40 μl. The master mix contained SYBR Green I Dye, 50 mM Tris-HCl pH8.3, 75 mM KCl, DMSO, Rox reference dye, 5 mM MgCl₂, 2 mM dNTP, Platinum Taq High Fidelity (1U/reaction), and 0.5 μM of each primer. The cDNA was diluted 1:36 from the synthesis reaction and eight microliters was used in each PCR reaction. For tissue distribution analyses, two microliters of cDNA from the Human Multiple Tissue and Human Immune System MTC cDNA panels were used as templates. The amplification program consisted of a 10 minute incubation at 95° C. followed by forty cycles of incubations at 95° C. for 15 seconds and 60° C. for 1 minute. Amplification was followed by melting curve analysis at 60° C. to demonstrate that the amplification was specific to a single amplicon. A negative control without cDNA template was run to assess the overall specificity.

A relative value for the initial target concentration in each reaction was determined using the TaqMan 5700 software. The threshold value was set to 0.5 to obtain cycle threshold values that were used to assign relative message levels for each target. The message levels of GAPDH were determined for each cDNA sample and were used to normalize all other genes tested from the same cDNA sample.

### Results

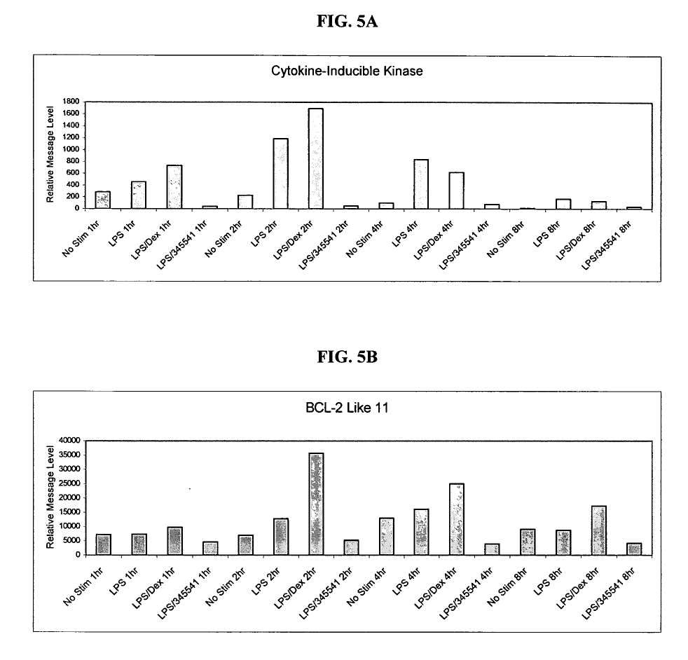

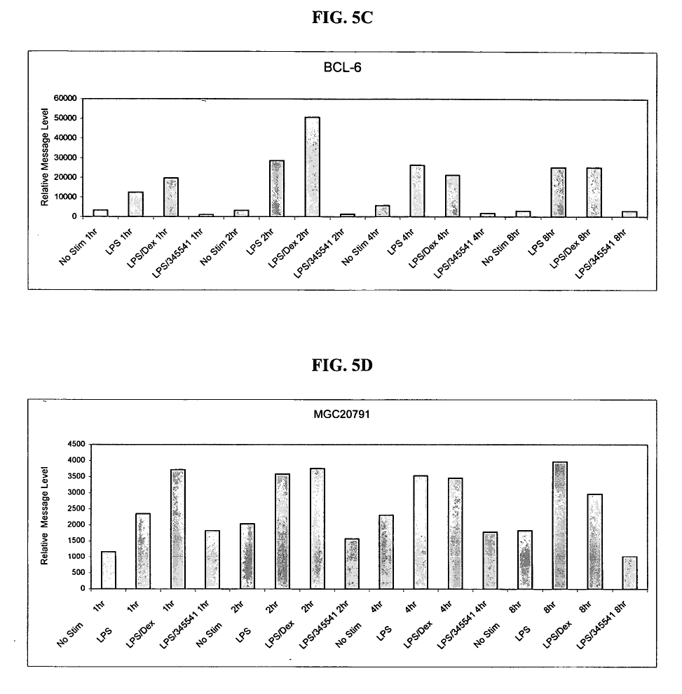

To further confirm the regulation of selected target genes, we used other inhibitors of the NF-κB activation pathway. Although it has other transcriptional effects, dexamethasone inhibits NF-κB activity via glucocorticoid receptor-mediated transrepression (Reichardt et al. (2001) EMBO J. 20:7168-7173). BMS-345541 is a selective IKK-2 inhibitor (BMS patent, Burke et al. (2003) J. Biol. Chem. 278:1450-1456). Induction of Cytokine-Inducible Kinase (CNK) expression was detected by 1 hour post stimulation and peaked at 2 hours (FIG. 5A). At all time points, addition of BMS-345541 potently inhibited expression of CNK. In contrast, addition of dexamethasone induced CNK mRNA levels above that detected with LPS stimulation alone.

Expression of BCL-2 Like 11 was modestly induced by LPS with levels peaking between 2 and 4 hours post stimulation (FIG. 5B). Addition of BMS-345541 significantly inhibited expression at each time point below that detected in resting cells. Addition of dexamethasone significantly increased BCL-2 like 11 expression above that detected in LPS stimulated cells alone.

Expression of BCL-6 peaked between 2 and 4 hours post stimulation (FIG. 5C). Similar to the other target genes, addition of BMS-345541 significantly inhibited the expression of BCL-6. Addition of dexamethasone failed to inhibit BCL-6 expression. At the 2 hour time point, dexamethasone significantly upregulated BCL-6 expression above that detected in LPS stimulated cells alone. Since dexamethasone is a glucocorticoid receptor agonist, some of these genes may contain glucocorticoid response elements in their promoters that override the effects of transrepression (Hofmann et al. (2002) Biol. Chem. 383:1947-1951).

Expression of MGC20791 was maximal by 2 hours post stimulation and remained high through 8 hours (FIG. 5D). Dexamethasone failed to inhibit LPS-mediated induction of MGC20791 mRNA. Addition of BMS-345541 significantly inhibited MGC20791 expression at all time points examined.

Expression of Stat1 mRNA did not significantly increase until 8 hours post stimulation (FIG. 5E). At this time point, both dexamethasone and BMS-345541 significantly inhibited Stat1 expression.

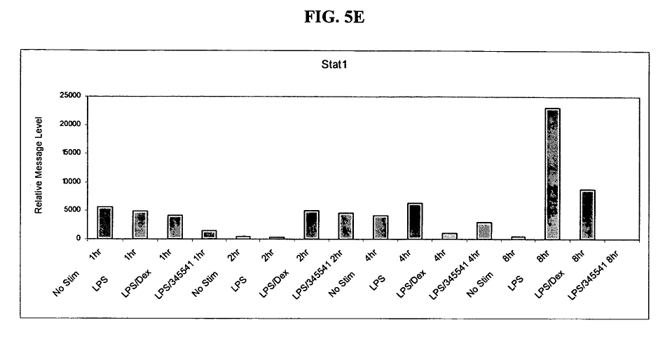

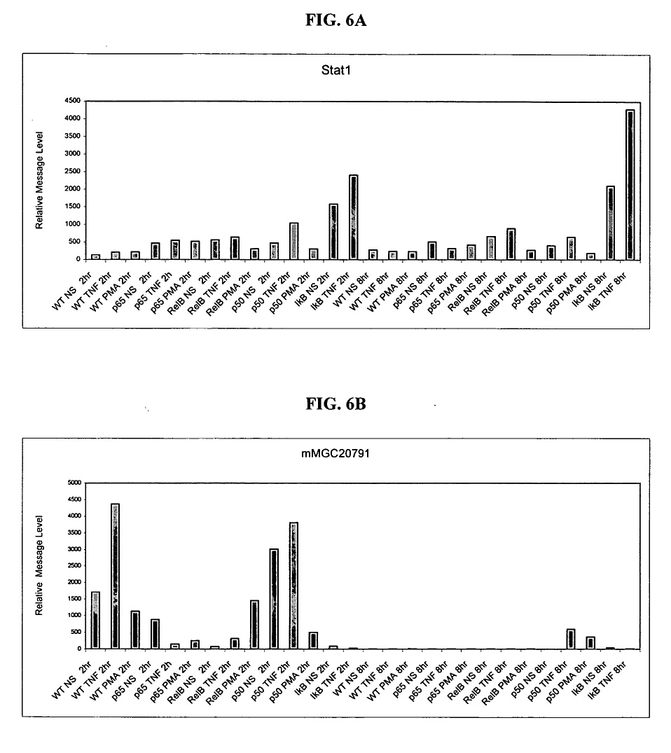

To confirm the NF-κB-dependent expression of selected target genes, we profiled their expression in mouse embryonic fibroblasts derived from germline knockouts of different NF-κB family members. Wild type 3T3 cells, and embryonic fibroblasts derived from germline knockouts of p65, RelB, p50, and IκBα were stimulated for 2 or 8 hours with either TNFα or PMA. At each time point, mRNA was isolated and real time PCR was performed. Stat1 expression was not induced in response to either TNFα or PMA (FIG. 6A). However, expression was superinduced in fibroblast lines deficient in IκBα, a negative regulator of NF-κB activity. This suggests that Stat1 expression can be regulated by NF-κB activity.

Expression of the mouse homologue of MGC20791 was induced in wild type fibroblasts in response to TNFα (FIG. 6B). No induction was observed in fibroblasts deficient for either p65 or RelB. However, TNFα-dependent expression was observed in fibroblasts deficient for p50, suggesting that NF-κB complexes containing p65 and RelB, but not p50, are required for MGC20791 induction.

High constitutive expression of BCL-6 was detected in wild type fibroblasts (FIG. 6C). No induction was observed in response to TNFα. Similar levels of expression were observed in fibroblasts deficient for p65 expression. However, significantly lower levels of mRNA were detected in fibroblasts derived from p50 knockout animals. These data suggest that NF-κB complexes containing p50 contribute to BCL-6 expression.

### Example 12

### Methods of Confirming the Associating of the MGC20791 Target to the NF-κB Pathway

### Reporter Assays

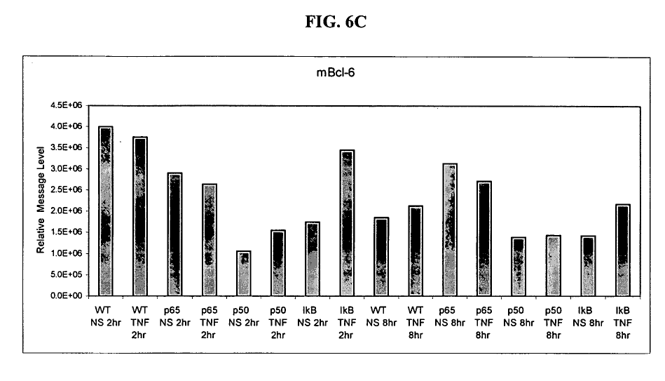

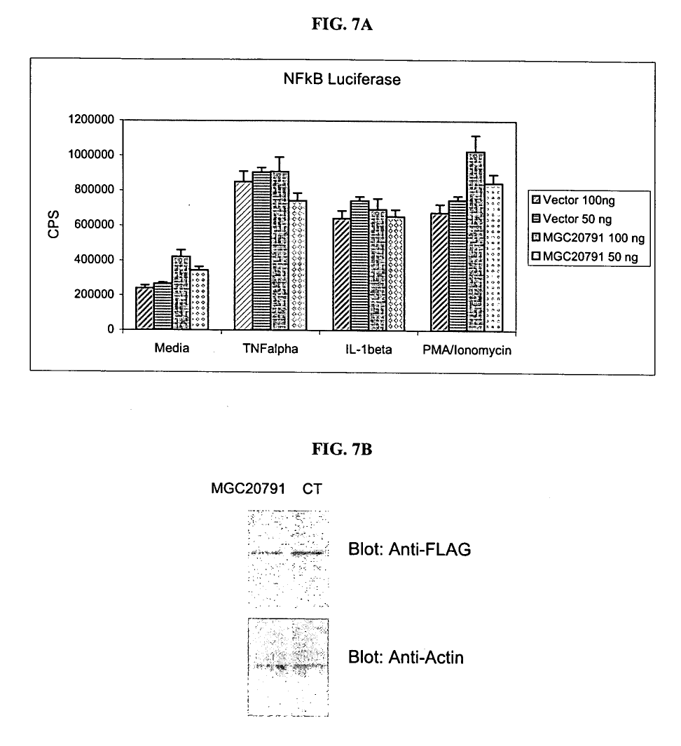

A549 cells were stably transfected with an NF-κB-luciferase reporter construct. The stable cell line was plated (10,000 cells/well) in 96 well white plates (Hewlett Packard) and transiently transfected using Lipofectamine 2000 (Invitrogen) with either the pcDNA3.1 vector (Invitrogen) or pcDNA3.1 containing the full length MGC20791 coding sequence and a FLAG® epitope tag. After an overnight culture, the complexes were removed, and cells were stimulated with either medium (RPMI without phenol red containing 10% FCS and glutamax), 10 ng/ml TNFα, 10 ng/ml IL-1β, or with 10 ng/ml PMA and 1 μg/ml ionomycin. After a 6 hour stimulation, luciferase substrate was added (Promega), and the signal was read on a Topcount (Hewlett Packard)—see FIG. 7A.

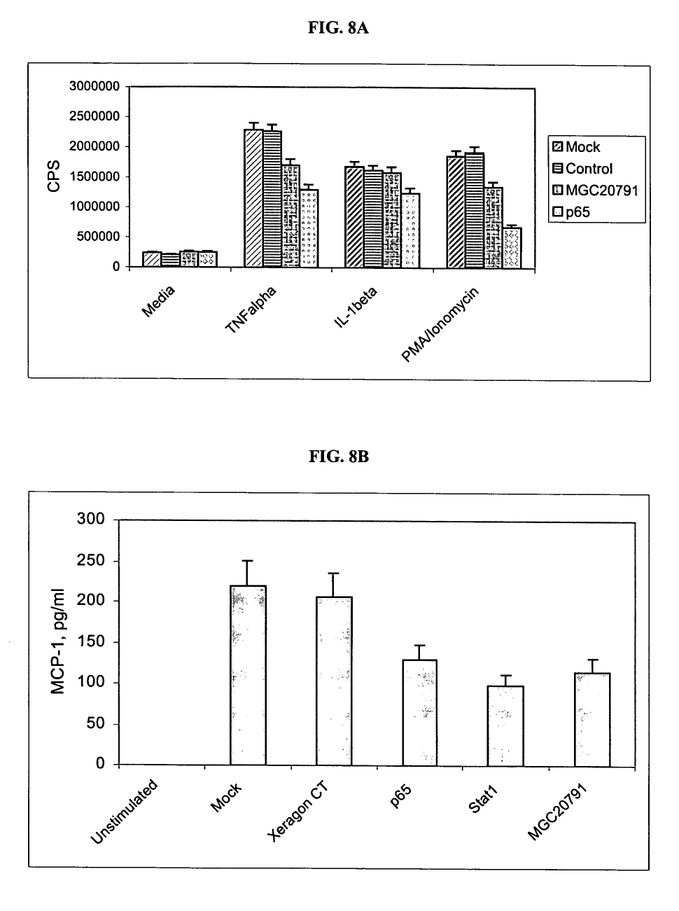

In some experiments, the A549 stable cell line containing the NF-κB reporter construct was transiently transfected as described above with siRNAs (100 nM, Sequitur) specific for either MGC20791, NF-κB p65, or a control sequence (see FIG. 8A). The cells were stimulated exactly as described above.

### siRNA Studies

To confirm knockdown of MGC20791 protein by siRNA reagents, Cos-7 cells were plated in 6-well plates at 300,000 cells/well. The cells were transfected with pcDNA3.1 encoding the FLAG®-tagged complete MGC20791 coding sequence. The cells were co-transfected with either control or MGC20791-specific siRNA duplexes (Sequitur, 100 nM). Following an overnight culture, the cells were harvested, lysed in buffer, and analyzed by Western blot with anti-FLAG® (Sigma) and anti-actin (Santa Cruz) antibodies (see FIG. 7B).

### Huvec Cytokine Assays

HUVECs were obtained from Clonetics (Cambrex, Walkersville, Md.). The cells were plated in 48-well plates (30,000 cells/well) in EG-2 media (Cambrex) and cultured overnight. The cells were transfected using Lipofectamine 2000 with siRNA duplexes specific for either MGC20791, NF-κB p65, Stat1, or control sequences. After an overnight culture, the duplexes were removed and the cells were stimulated with 10 ng/ml TNFα. Following a 6-hour stimulation, supernatants were removed for ELISA analyses. The cells were cultured with media and MTS reagent (Promega) for an additional two hours to measure cell viability. Concentrations of IL-6 and IL-8 in the supernatants were measured by ELISA (BD Pharmingen). Values were normalized to cell viability using the MTS results (see FIG. 8B).

### Results

One of the NF-κB associated polypeptide of the present invention that was isolated from the subtraction library described herein, MGC20791 (NM_{—}052864) has recently been described as a novel TRAF2 binding protein involved in TNF and IL-1 signaling pathways (Kanamori et al. (2002) Bioch. Biophys. Res. Comm. 290:1108-1113; Takatsuna et al. (2003) J. Biol. Chem. 278:12144-12150; Matsuda et al. (2003) Oncogene 22:3307-3318). Similar to these reports, the inventors observed an increase in NF-κB-dependent transcriptional activity when MGC20791 was overexpressed in an A549 cell line containing a stably integrated NF-κB reporter construct (see FIG. 7A). Overexpression of MGC20791 did not significantly affect the responses to either TNFα or IL-1β. However, PMA/Ionomycin-induced activation of NF-κB was significantly increased when MGC20791 was overexpressed.

Consistent with the published reports, and the above experiment, partial knockdown of MGC20791 protein using siRNA (see FIG. 7B) decreased TNFα-induced and PMA/Ionomycin-induced NF-κB activation in the A549 stable reporter line (see FIG. 8A). Protein knockdown had no effect on IL-1β-dependent activity.

To examine the role of MGC20791 in a more physiologic NF-κB-dependent response, the inventors tested the effect of MGC20791 knockdown on TNFα-induced MCP-1 production by human umbilical vein endothelial cells (HUVECs, see FIG. 8B). Transfection of HUVECs with siRNA specific for MGC20791 significantly inhibited TNFα-dependent MCP-1 secretion. The inhibition seen was similar to that achieved by knocking down either the p65 subunit of NF-κB or the transcription factor Stat1. Both transcription factors are known to be required for MCP-1 production. In summary, these experiments suggest that the NF-κB target polypeptide MGC20791 functions in NF-κB dependent responses and therefore could represent an important therapeutic target for the treatment of inflammatory diseases.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

### BRIEF DESCRIPTION OF THE SEQUENCE LISTING

Incorporated herein by reference in its entirety is a Sequence Listing, including SEQ ID NO: 1 through SEQ ID NO:823. The Sequence Listing is contained on a compact disc, i.e., CD-ROM, three identical copies of which are filed herewith. The Sequence Listing, in IBM/PC MS-DOS format (named “D0284.NP.ST25.txt”), PatentIn Version 3.2, was recorded on Jan. 13, 2004, and is 2,680 kilobytes in size.

What is claimed is:

1. An isolated nucleic acid molecule comprising a polynucleotide having a nucleotide sequence selected from the group consisting of:
(a) a polynucleotide of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(b) a polynucleotide encoding a polypeptide of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, which is hybridizable to SEQ ID NOS: (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(c) a polynucleotide encoding a polypeptide domain of SEQ ID-NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780 which is hybridizable to SEQ ID NOS: (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(d) a polynucleotide encoding a polypeptide epitope of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, which is hybridizable to SEQ ID NOS: (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(e) a polynucleotide encoding a polypeptide of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780 which is hybridizable to SEQ ID NOS: (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); having NF-κB modulating activity;
(f) a polynucleotide which is a variant of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(g) a polynucleotide which is an allelic variant of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, -81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 3.19, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(h) a polynucleotide which encodes a species homologue of the (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(i) a polynucleotide which represents the complimentary sequence (antisense) of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); and
(j) a polynucleotide capable of hybridizing under stringent conditions to any one of the polynucleotides specified in (a)-(i), wherein said polynucleotide does not hybridize under stringent conditions to a nucleic acid molecule having a nucleotide sequence of only A residues or of only T residues.

2. The isolated nucleic acid molecule of claim 1, wherein the polynucleotide comprising a nucleotide sequence encoding a NF-κB modulatory protein, or fragment thereof.

3. The isolated nucleic acid molecule of claim 1, wherein the polynucleotide comprising a nucleotide sequence encoding the sequence identified as (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); which is hybridizable to (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823).

4. The isolated nucleic acid molecule of claim 1, wherein the polynucleotide comprising the entire nucleotide sequence of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); which is hybridizable to (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823).

5. The isolated nucleic acid molecule of claim 2, wherein the nucleotide sequence consisting of sequential nucleotide deletions from either the C-terminus or the N-terminus.

6. An isolated polypeptide comprising an amino acid sequence selected from the group consisting of:
(a) a polypeptide of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780;
(b) a polypeptide of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, capable of modulating an NF-κB response;
(c) a polypeptide domain of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780;
(d) a polypeptide epitope of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, -148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780;
(e) a full length protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780;
(f) a variant of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780;
(g) an allelic variant of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 1 10, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780; and
(h) a species homologue of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780.

7. The isolated polypeptide of claim 6, wherein the full length protein comprises sequential amino acid deletions from either the C-terminus or the N-terminus.

8. An isolated antibody that binds specifically to the isolated polypeptide of claim 6.

9. A method for preventing, treating, or ameliorating a medical condition, comprising administering to a mammalian subject a therapeutically effective amount of the polypeptide of claim 6.

10. A method of diagnosing a NF-κB associated condition or a susceptibility to a NF-κB associated condition in a subject wherein said condition is a member of the group consisting of an immune disorder; an inflammatory disorder in which polypeptides of the present invention are associated with the disorder either directly; or indirectly; an inflammatory disorder related to aberrant NF-κB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; necrotic lesions; wounds; organ transplant rejection; conditions related to organ transplant rejection; disorders related to aberrant signal transduction; proliferating disorders; cancers; and HIV propagation in cells infected with other viruses; comprising:
(a) determining the presence or absence of a mutation in the polynucleotide of claim 1; and
(b) diagnosing a NF-κB associated condition or a susceptibility to a NF-κB associated condition based on the presence or absence of said mutation, wherein said mutation indicates a predisposition to at least one of said NF-κB associated disorders

11. A method of diagnosing an NF-κB associated condition or a susceptibility to a NF-κB associated condition in a subject wherein said condition is a member of the group consisting of an immune disorder; an inflammatory disorder in which polypeptides of the present invention are associated with the disorder either directly, or indirectly; an inflammatory disorder related to aberrant NF-κB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; necrotic lesions; wounds; organ transplant rejection; conditions related to organ transplant rejection; disorders related to aberrant signal transduction; proliferating disorders; cancers; and HIV propagation in cells infected with other viruses, comprising:
(a) determining the presence or amount of expression of the polypeptide of claim 6 in a biological sample; and
(b) diagnosing a NF-κB associated condition or a susceptibility to a pathological condition based on the presence or amount of expression of the polypeptide.

12. A method for identifing a binding partner to the polypeptide of claim 6 comprising:
(a) contacting the polypeptide of claim 6 with a binding partner; and
(b) determining whether the binding partner effects an activity of the polypeptide.

13. The method for preventing, treating, or ameliorating a medical condition of claim 9, wherein the medical condition is a member of the group consisting of an immune disorder; an inflammatory disorder in which polypeptides of the present invention are associated with the disorder either directly; or indirectly; an inflammatory disorder related to aberrant NF-κB regulation; a cancer; aberrant apoptosis; hepatic disorders; Hodgkins lymphomas; hematopoietic tumors; hyper-IgM syndromes; hypohydrotic ectodermal dysplasia; X-linked anhidrotic ectodermal dysplasia; Immunodeficiency; al incontinentia pigmenti; viral infections; HIV-1; HTLV-1; hepatitis B; hepatitis C; EBV; influenza; viral replication; host cell survival; and evasion of immune responses; rheumatoid arthritis inflammatory bowel disease; colitis; asthma; atherosclerosis; cachexia; euthyroid sick syndrome; stroke; EAE; autoimmune disorders; disorders related to hyper immune activity; disorders related to aberrant acute phase responses; hypercongenital conditions; birth defects; necrotic lesions; wounds; organ transplant rejection; conditions related to organ transplant rejection; disorders related to aberrant signal transduction; proliferating disorders; cancers; and HIV propagation in cells infected with other viruses, comprising administering an effect amount of a modulator of a polypeptide selected from the group consisting of: SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, to treat, ameliorate, or detect said disorder.

14. A method of identifying a compound that modulates the biological activity of a NF-κB associated molecule, comprising:
(a) combining a candidate modulator compound with a NF-κB associated molecule having the sequence set forth in a member of the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, or a polypeptide encoded by a polynucleotide selected from the group consisting of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); and
(b) measuring an effect of the candidate modulator compound on the activity of the NF-κB associated molecule.

15. A method of identifying a compound that modulates the biological activity of an NF-κB associated molecule, comprising:
(a) combining a candidate modulator compound with a host cell expressing a NF-κB associated molecule having the sequence as set forth in a member of the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 748, 750, 752, 754, 756, 758, 760, 762, 764, 766, 768, 770, 772, 774, 776, 778, & 780, or a polypeptide encoded by a polynucleotide selected from the group consisting of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); and
(b) measuring an effect of the candidate modulator compound on the activity of the expressed NF-κB associated molecule.

16. A method of identifying a compound that modulates the biological activity of a NF-κB associated molecule, comprising:
(a) combining a candidate modulator compound with a host cell containing a vector comprising the polynucleotide sequence selected from the group consisting of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823); wherein a NF-κB associated molecule is expressed by the cell; and
(b) measuring an effect of the candidate modulator compound on the activity of the expressed NF-κB associated molecule.

17. A method of screening for a compound that is capable of modulating the biological activity of a NF-κB associated molecule, comprising the steps of:
(a) providing a host cell containing a vector comprising the polynucleotide sequence selected from the group consisting of (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, ′ 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 444, 446, 448, 450, 452, 454, 456, 458, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 530, 532, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 612, 614, 616, 618, 620, 622, 624, 626, 628, 630, 632, 634, 636, 638, 639, 641, 643, 645, 647, 649, 651, 653, 655, 657, 659, 661, 663, 665, 667, 669, 671, 673, 675, 747, 749, 751, 753, 755, 757, 759, 761, 763, 765, 767, 769, 771, 773, 775, 777, 779 & 823);
(b) determining the biological activity of the NFκRB associated molecule in the absence of a modulator compound;
(c) contacting the cell with the modulator compound; and
(d) determining the biological activity of the NF-κB associated molecule in the presence of the modulator compound; wherein a difference between the activity of the NF-κB associated molecule in the presence of the modulator compound and in the absence of the modulator compound indicates a modulating effect of the compound.

18. A compound that modulates the biological activity of a human NF-κB associated molecule as identified by the method according to a member of the group consisting of: the compound(s) identified according to the method of claim 14; the compound(s) identified according to the method of claim 15; the compound(s) identified according to the method of claim 16; and the compound(s) identified according to the method of claim 17.
